(19) United States
(12) Patent Application Publication
(10) Pub. No.: US 2024/0409995 A1
(43) Pub. Date: Dec. 12, 2024

(54) SINGLE-MOLECULE PEPTIDE SEQUENCING THROUGH MOLECULAR BARCODING AND EX-SITU ANALYSIS

(71) Applicant: Glyphic Biotechnologies, Inc., New York, NY (US)

(72) Inventors: Daniel Masao ESTANDIAN, Antioch, CA (US); Asmamaw WASSIE, New York, NY (US); Boyang HUA, Oakland, CA (US); Jai PRAKASH, Fremont, CA (US)

(21) Appl. No.: 18/743,649

(22) Filed: Jun. 14, 2024

Related U.S. Application Data
(63) Continuation of application No. PCT/US2022/081392, filed on Dec. 12, 2022.
(60) Provisional application No. 63/289,261, filed on Dec. 14, 2021.
(60) Provisional application No. 63/326,382, filed on Apr. 1, 2022.

(51) Int. Cl. C12Q 1/6869; C12Q 1/25; C12Q 1/48; C12Q 1/6806; C12Q 1/6825; G01N 21/64

(52) U.S. Cl. CPC C12Q 1/6869; C12Q 1/25; C12Q 1/485; C12Q 1/6806; C12Q 1/6825; G01N 21/6456; G01N 21/6486; G01N 2333/9015; G01N 2333/9126

(57) ABSTRACT

The present disclosure provides systems and methods directed towards single molecule protein sequencing, through use of a barcode transfer reagent. Systems and methods described herein allow for massively parallel single-molecule protein sequencing through molecular barcoding and ex-situ analysis.

### CROSS-REFERENCE

This application is a continuation application of International Application PCT/US2022/081392, filed on Dec. 12, 2022, which claims priority to United States Provisional Patent Application Numbers 63/289,261, filed on Dec. 14, 2021, and 63/326,382, filed on Apr. 1, 2022, each of which applications is incorporated herein in its entirety.

### SEQUENCE LISTING

The instant application contains a Sequence Listing which has been submitted electronically in XML file format and is hereby incorporated by reference in its entirety. Said XML copy, created on Aug. 16, 2024, is named 60652_702_301_SL.xml and is 14,141 bytes in size.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BACKGROUND

While the emergence of sensitive and high-throughput DNA sequencing technologies has opened the door for studying cellular genomes and gene expression profiles, there exist no analogous approaches for studying the proteome. This need is even more urgent, because much of the regulation and diversity related to the emergence of diseases occurs at the proteome level. Development of single-molecule protein sequencing (SMPS) will have an immediate and profound impact on genomic and proteomic studies of normal and disease conditions, including cancer, infection, and immunity.

Existing approaches for single molecule protein sequencing require the identification of amino acids using binding agents or fluorophores within their native protein environment. This arrangement results in inefficiencies since amino acids (<Inm) are less than 0.3 nm from variable adjacent amino acids and possibly sequestered inside of the protein due to protein folding. Thus, protein properties block the large bulky identification tools such as proteins (5-10 nm), binding agents (5-15 nm), or fluorophores (1-5 nm) used to label them.

### SUMMARY

Considering the present need for improved methods of single molecule protein sequencing, provided herein are methods, compositions, and systems to address these needs. The present disclosure provides methods, compositions, and systems that comprise separating amino acids from proteins or peptides, and then identifying them ex-situ, thereby overcoming issues with bulky identification tools and internally-sequestered amino acids.

In an aspect, provided herein is a method for sequencing a peptide comprising: (a) providing a peptide coupled to a barcode; (b) contacting a Barcode Transfer Reagent (BTR) to a terminal amino acid or a terminal amino acid derivative of the peptide, wherein the BTR and the terminal amino acid or the terminal amino acid derivative generate a barcoded-amino acid complex (BTR-AC) comprising barcode information; (c) cleaving the BTR-AC from the peptide to release the BTR-AC; (d) repeating steps (b) to (c) at least once to generate a plurality of BTR-ACs; (e) contacting the plurality of BTR-ACs with a binding agent; and (f) reading out barcode information from the BTR-ACs, thereby sequencing the terminal amino acids.

In some embodiments, the peptide or the protein is from a biological sample. In some embodiments, the biological sample is a cell suspension, a culture of cells, a tissue sample, a bodily fluid, or an environmental sample. In some embodiments, the tissue sample comprises a biopsy. In some embodiments, the bodily fluid comprises whole blood, serum, plasma, urine, saliva, stool, lavage, or cerebrospinal fluid. In some embodiments, the environmental sample comprises a sewage sample. In some embodiments, the biological sample is treated to de-aggregate the protein. In some embodiments, the biological sample is not treated to de-aggregate the protein. In some embodiments, the biological sample is sorted to isolate a specific cell type. In some embodiments, the specific cell type is an immune cell.

In some embodiments, the barcode comprises DNA or RNA. In some embodiments, the barcode comprises a peptide barcode or a protein barcode. In some embodiments, the peptide barcode or the protein barcode is covalently attached to the peptide. In some embodiments, the barcode provides barcode information, the barcode information comprising: multiplexing information, temporal information, proximity information, order information, structural information, interactional information, or molecular type information. In some embodiments, the barcode further comprises a hairpin segment. In some embodiments, the barcode comprises one or more artificial nucleic acids. In some embodiments, the one or more artificial nucleic acids are locked-nucleic acids (LNA) or its derivatives. In some embodiments, the one or more artificial nucleic acids are peptide nucleic acids (PNA) or its derivatives. In some embodiments, the one or more artificial nucleic acids are hexitol nucleic acids (HNA) or its derivatives. In some embodiments, the one or more artificial nucleic acids are cyclohexane nucleic acids (CeNA) or its derivatives.

In some embodiments, the barcode comprises a peptide. In some embodiments, the barcode comprises a chemical polymer. In some embodiments, the barcode comprises a heavy metal tag. In some embodiments, the barcode is coupled to the peptide or protein at an N-terminal amino acid, a C-terminal amino acid, or an internal amino acid.

In some embodiments, the method further comprises, subsequent to (a), performing nucleic acid-based amplification to copy the barcode to one or more further locations of the peptide or protein. In some embodiments, subsequent to (a), the peptide or protein is encapsulated in a partition with a set of barcodes, wherein the set of barcodes are configured to label the peptide or protein at multiple sites. In some embodiments, the barcode is coupled to the protein via a substrate.

In some embodiments, the peptide or protein is obtained from a biological sample in a partition.

In some embodiments, the method further comprises tagging the peptide or protein with a chemical moiety to generate a barcoded peptide or protein. In some embodiments, the method further comprises tagging the peptide or protein with a DNA barcode to generate a barcoded peptide or protein. In some embodiments, the method further comprises tagging the barcoded peptide or protein with a spatial barcode. In some embodiments, the method further comprises incorporating the barcoded peptide or protein into a hydrogel to preserve the position of the peptide or protein in the sample. In some embodiments, the method further comprises, prior to (a), attaching the barcode to the N-terminal amino acid of said peptide or protein. In some embodiments, attaching comprises employing amide coupling to the N-terminal amino acid. In some embodiments, attaching comprises contacting the N-terminal amino acid with 2-pyridinecarboxaldehyde or a derivative thereof. In some embodiments, the method further comprises, prior to (a), attaching the barcode to the C-terminal amino acid. In some embodiments, attaching comprises amide coupling to a C-terminus carboxylic group of the C-terminal amino acid. In some embodiments, attaching comprises photoredox tagging of a C-terminus carboxylic group of the C-terminal amino acid. In some embodiments, the method further comprises, prior to (a), attaching the barcode to the internal amino acid in the peptide. In some embodiments, attaching comprises amide coupling. In some embodiments, attaching comprises performing an alkylation reaction. In some embodiments, attaching comprises linking the barcode to the internal amino acid through disulfide bridge labeling of cysteines.

In some embodiments, the barcode is conjugated to a microbead. In some embodiments, the barcode is conjugated to a bulk surface support. In some embodiments, the barcode is in a solution.

In some embodiments, the BTR is conjugated to the N-terminal amino acid or the C-terminal amino acid of the peptide. In some embodiments, the BTR is conjugated to the N-terminal amino acid of the peptide. In some embodiments, the BTR is conjugated to the C-terminal amino acid of the peptide. In some embodiments, the BTR is conjugated to a substrate. In some embodiments, the peptide or protein is conjugated to a bulk surface support. In some embodiments, the bulk surface support is a microbead or a glass slide. In some embodiments, the peptide or protein is conjugated to the bulk surface support via a N-terminal amino acid. In some embodiments, the peptide or protein is conjugated to the bulk surface support via a C-terminal amino acid. In some embodiments, the peptide or protein is conjugated to the bulk surface support via an internal amino acid. In some embodiments, conjugating the peptide or protein to the bulk surface support comprises performing a chemical reaction. In some embodiments, conjugating the peptide or protein to the bulk surface support comprises performing an enzymatic reaction. In some embodiments, the enzymatic reaction is performed by Sortase A, Subtiligase, Butelase I, trypsiligase, or ubiquitin ligase. In some embodiments, the enzymatic reaction comprises a modified substrate. In some embodiments, the modified substrate comprises a linker. In some embodiments, the enzymatic reaction comprises attaching the linker to the peptide or protein. In some embodiments, the linker attached to the peptide or protein conjugates to a surface. In some embodiments, the linker is reactive. In some embodiments, the linker covalently conjugates to a surface. In some embodiments, the linker is enzymatically conjugated to a surface.

In some embodiments, the method further comprises transferring the barcode from the peptide to the BTR. In some embodiments, transferring comprises conjugating the barcode to the BTR via polymerase extension. In some embodiments, transferring comprises ligating to the BTR and cleaving. In some embodiments, transferring comprises recombination. In some embodiments, transferring comprises Toehold Mediated Strand Displacement and ligation.

In some embodiments, cleaving the BTR-AC comprises a chemical cleavage. In some embodiments, the chemical cleavage is an acidic cleavage or a basic cleavage. In some embodiments, cleaving the BTR-AC comprises an enzymatic cleavage. In some embodiments, cleaving the BTR-AC comprises a catalytical cleavage.

In some embodiments, the binding agent comprises an antibody. In some embodiments, the binding agent comprises a nanobody. In some embodiments, the binding agent comprises a modified amino acyl tRNA transferase. In some embodiments, the binding agent comprises an artificial protein domain. In some embodiments, the binding agent comprises an aptamer. In some embodiments, the binding agent comprises an aminopeptidase or a carboxypeptidase. In some embodiments, the binding agent comprises a modified endoprotease. In some embodiments, the binding agent recognizes an individual amino acid. In some embodiments, the binding agent recognizes a specific dipeptide. In some embodiments, the binding agent recognizes a specific tripeptide. In some embodiments, the binding agent recognizes a post-translational modification (PTM). In some embodiments, the binding agent is conjugated to a microbead. In some embodiments, the binding agent is in a solution. In some embodiments, the binding agent comprises a barcode associated with a specific amino acid. In some embodiments, the binding agent comprises a barcode associated with a specific post-translational modification. In some embodiments, one or more binding agents are linked or fused together to create a multimeric binding agent. In some embodiments, the multimeric binding agent recognizes the sum of the individual component binding agent's binding interactions. In some embodiments, the binding agent comprises a binding agent barcode and further comprising, copying the barcode of the peptide or protein to the binding agent barcode, thereby extending the binding agent barcode to generate an extended barcode. In some embodiments, the binding agent comprises a binding agent barcode and the method further comprises copying the binding agent barcode to the peptide barcode, thereby extending the peptide barcode to generate an extended barcode. In some embodiments, the method further comprises amplifying the extended barcode. In some embodiments, the method further comprises amplifying the extended barcode via PCR. In some embodiments, the method further comprises sequencing the extended barcode.

In some embodiments, the binding agent comprises a binding agent barcode and further comprising, ligating the binding agent barcode to the barcode of the peptide to generate a ligated barcode. In some embodiments, the method further comprises ligating the peptide barcode to the binding agent barcode to generated a ligated barcode. In some embodiments, the method further comprises amplifying the ligated barcode. In some embodiments, the method further comprises amplifying the ligated barcode via PCR. In some embodiments, the method further comprises sequencing the ligated barcodes.

In some embodiments, (f) comprises sequencing the barcode information of the BTR-ACs via Next Generation Sequencing (NGS). In some embodiments, the method further comprises amplifying the BTR-AC or portion thereof. In some embodiments, the method further comprises sequencing the barcode associated with the terminal amino acid of the peptide. In some embodiments, (f) comprises a sequencing by synthesis approach. In some embodiments, the sequencing by synthesis approach comprises an Illumina Sequencer or a PacBio sequencer. In some embodiments, (f) comprises a sequencing by ligation approach.(f) comprises a nanopore based sequencing approach. In some embodiments, (f) comprises a sequence hybridization approach. In some embodiments, (f) comprises a ligation-based approach.

In some embodiments, the method further comprises generating barcode reads from reading out the barcode information and assembling the barcode reads from (f) into a peptide sequence. In some embodiments, the method further comprises assembling the barcode reads into the peptide sequence by a computational De-Novo Assembly. In some embodiments, the method further comprises assembling the barcode reads into the peptide sequence by a computational Reference Based Assembly. In some embodiments, the method further comprises mapping the barcode reads to a known proteome database.

In another aspect, provided herein is a Barcode Transfer Reagent (BTR) comprising: a primer sequence that binds to a site on a barcode; and a chemical moiety that reacts with either a N-terminal amino acid, a C-terminal amino acid, or both terminal amino acids of a barcoded peptide.

In some embodiments, the BTR further comprises (c) a sequence with cycle information. In some embodiments, the sequence with cycle information comprises DNA, RNA, HNA, CeNA, modified nucleotides, protein, or synthetic materials. In some embodiments, the sequence with cycle information comprises DNA. In some embodiments, the sequence with cycle information comprises a peptide. In some embodiments, the primer sequence comprises RNA, DNA, HNA, CeNA or mixtures thereof. In some embodiments, the primer sequence comprises RNA. In some embodiments, the primer sequence comprises DNA. In some embodiments, the primer sequence comprises modified nucleotides.

In some embodiments, the chemical moiety reacts with a N-terminal amino acid of the peptide. In some embodiments, the chemical moiety comprises phenyl isothiocyanate (PITC), dinitrofluorobenzene (DNFB), dansyl chloride, or isothiocyanate. In some embodiments, the chemical moiety reacts with a C-terminal amino acid of the peptide. In some embodiments, the chemical moiety comprises thiocyanate or isothiocyanate. In some embodiments, the chemical moiety reacts with a N-terminal amino acid and a C-terminal amino acid of the peptide.

In some embodiments, the BTR is conjugated to the N-terminal amino acid of the peptide. In some embodiments, the BTR is conjugated to the barcoded peptide with a conjugation chemistry. In some embodiments, the BTR is conjugated to the barcoded peptide using Click chemistry. In some embodiments, the BTR is conjugated to the barcoded peptide with a thiol Chemistry. In some embodiments, the BTR is conjugated to the barcoded peptide with an amine Chemistry.

In some embodiments, the barcode on the barcoded peptide is transferred to the BTR. In some embodiments, the barcode is transferred to the BTR via polymerase extension. In some embodiments, the barcode is transferred to the BTR via ligation and cleavage. In some embodiments, the barcode is transferred to the BTR via recombination. In some embodiments, the barcode is transferred to the BTR via Toehold Mediated Strand Displacement and Ligation.

In some embodiments, the terminal amino acid of the peptide is cleaved to remove the BTR-AC. In some embodiments, the BTR-AC comprises a chemical cleavage. In some embodiments, the chemical cleavage comprises an acidic cleavage or a basic cleavage. In some embodiments, the BTR-AC comprises an enzymatical cleavage. In some embodiments, the BTR-AC comprises a catalytical cleavage.

In another aspect, provided herein is a method comprising: (a) fixing a sample comprising a peptide; (b) permeabilizing and digesting the sample; (c) transferring the peptide to an array; (d) tagging the peptide with a plurality of barcodes to generate a tagged peptide; (e) imaging the peptide or extending a barcode of the plurality of barcodes to a neighboring barcode; (f) releasing the tagged peptide from the array; and (g) collecting the tagged peptide for further processing.

In some embodiments, a spatial location of the peptide is 2D or 3D. In some embodiments, spatial location of the peptide is 2D. In some embodiments, the method further comprises determining the 2D spatial location with a 2D spatial array. In some embodiments, the 2D spatial array comprises a bead array. In some embodiments, the 2D spatial array comprises a printed DNA array. In some embodiments, the plurality of barcodes encodes a 2D location in the array.

In some embodiments, fixing the sample comprises use of formaldehyde. In some embodiments, permeabilizing the sample comprises use of a detergent.

In some embodiments, the method further comprises digesting the sample to release the peptide. In some embodiments, digesting the peptide comprises a heat denaturation. In some embodiments, digesting the peptide comprises an enzymatic digestion.

In some embodiments, the method further comprises conjugating the peptide to the plurality of barcodes on the array. In some embodiments, the plurality of barcodes comprises two-photon photoreactive chemical groups. In some embodiments, the method further comprises imaging a barcode of the plurality of barcodes to locate the peptide in the sample. In some embodiments, the method further comprises releasing the tagged peptide from the 2D array. In some embodiments, the method further comprises releasing the tagged peptide from the 2D array via an endonuclease cleavage. In some embodiments, the method further comprises releasing the tagged peptide via a chemical release. In some embodiments, the method further comprises sequencing the tagged peptide. In some embodiments, sequencing comprises conjugating a barcode transfer reagent (BTR) comprising barcode information to a terminal amino acid or a terminal amino acid derivative of the tagged peptide to generate a BTR-AC. In some embodiments, the method further comprises cleaving the BTR-AC from the peptide to release the BTR-AC. In some embodiments, the method further comprises sorting the BTR-AC into a group. In some embodiments, the method further comprises sorting the BTR-AC into groups based on binding to a binding agent. In some embodiments, the method further comprises reading the barcode information from the BTR-AC, thereby determining the spatial location of the terminal amino acid.

In some embodiments, a spatial location of the peptide is 3D. In some embodiments, the method further comprises fixing the sample with formaldehyde. In some embodiments, the method further comprises embedding the sample in a hydrogel. In some embodiments, the method further comprises permeabilizing the sample. In some embodiments, the method further comprises digesting the sample to transfer the peptide to the array, wherein the array is a hydrogel array. In some embodiments, digesting the peptide comprises a heat denaturation. In some embodiments, digesting the peptide comprises an enzymatic digestion. In some embodiments, conjugating the peptide to the plurality of barcodes, wherein the conjugating comprises generating a covalent bond between the peptide and the plurality of barcodes. In some embodiments, the plurality of barcodes encodes a 3D location in the array, wherein the array is a hydrogel array. In some embodiments, the plurality of barcodes comprises two-photon photoreactive chemical groups. In some embodiments, the method further comprises imaging the tagged peptide to locate the peptide in the sample. In some embodiments, the method further comprises amplifying the plurality of barcodes in situ within the array. In some embodiments, the method further comprises sequencing the plurality of barcodes in the array, thereby determining the location of the peptide.

In some embodiments, the method further comprises releasing the tagged peptide from the array. In some embodiments, the method further comprises releasing the tagged peptide via an endonuclease cleavage. In some embodiments, the method further comprises releasing the tagged peptide via a chemical release.

In some embodiments, the method further comprises sequencing the tagged peptide. In some embodiments, the method further comprises conjugating a BTR comprising barcode information to a terminal amino acid of the peptide generate a barcoded-amino acid complex (BTR-AC). In some embodiments, the method further comprises cleaving the BTR-AC from the peptide to release the BTR-AC. In some embodiments, the method further comprises sorting the BTR-AC into a group. In some embodiments, the method further comprises sorting the BTR-AC into groups based on binding to an binding agent. In some embodiments, the method further comprises reading the barcode information from the BTR-AC, thereby determining the spatial location of the terminal amino acid. In some embodiments, the method further comprises sequencing the tagged peptide with Next Generation sequencing. In some embodiments, the method further comprises amplifying the sample. In some embodiments, the method further comprises sequencing the barcode associated with the terminal amino acid of the peptide. In some embodiments, (g) comprises sequencing, wherein the sequencing is a sequencing by synthesis approach. In some embodiments, sequencing by synthesis approach comprises using an Illumina Sequencer or a PacBio sequencer. In some embodiments, (g) comprises a sequencing by ligation approach. In some embodiments, (g) comprises a nanopore based sequencing approach. In some embodiments, (g) comprises a sequence hybridization approach. In some embodiments, (g) comprises a ligation-based approach.

In another aspect, disclosed herein is a method comprising (a) converting an amino acid or a post-translational modification on a peptide to a chemical group; (b) tagging the peptide with a barcode; (c) contacting a Barcode Transfer Reagent (BTR) to a terminal amino acid or a terminal amino acid derivative of the peptide, wherein the BTR and the terminal amino acid or the terminal amino acid derivative generate a barcoded-amino acid complex (BTR-AC); (d) cleaving the BTR-AC from the peptide to release the BTR-AC; and (e) detecting binding of a binding agent to the chemical group thereby detecting the amino acid or post-translational modification.

In some embodiments, the chemical group comprises an affinity tag. In some embodiments, the affinity tag is a peptide. In some embodiments, the affinity tag is a fluorophore. In some embodiments, the affinity tag is a hapten. In some embodiments, the affinity tag is composed of nucleic acids. In some embodiments, the affinity tag is a polymer.

In some embodiments, the binding agent is a multimeric binding agent comprising a plurality of binding agents that are linked or fused together.

In some embodiments, the post-translational modification comprises phosphorylation, acetylation, methylation, formylation, glycosylation, or ubiquitination. In some embodiments, converting the amino acids or post-translational modification is performed using a chemical or an enzymatic reaction.

In some embodiments, binding the binding agent to the BTR-AC or portion thereof. In some embodiments, the binding agent is an affinity reagent. In some embodiments, binding the affinity reagent to the chemical group. In some embodiments, the binding agent comprises an antibody. In some embodiments, the binding agent comprises a nanobody. In some embodiments, the binding agent comprises a modified amino acyl tRNA transferase. In some embodiments, the binding agent comprises an artificial protein domain. In some embodiments, the binding agent comprises an aptamer. In some embodiments, the binding agent comprises an aminopeptidase or a carboxypeptidase. In some embodiments, the binding agent comprises a modified endoprotease. In some embodiments, the binding agent has a barcode associated with a specific post-translational modification. In some embodiments, the binding agent has a barcode associated with a specific amino acid. In some embodiments, the binding agent is conjugated to a fluorophore or a hapten. In some embodiments, the binding agent is conjugated to a fluorophore. In some embodiments, the binding agent is conjugated to a hapten. In some embodiments, the binding agent comprises a binding agent barcode and the method further comprises, (f) sequencing the binding agent barcode, thereby detecting the post-translational modification.

In some embodiments, (f) comprises using a Next Generation Sequencing (NGS) platform. In some embodiments, the method further comprises amplifying the sample. In some embodiments, (f) comprises a sequencing by synthesis approach. In some embodiments, the sequencing by synthesis approach comprises using an Illumina Sequencer or a PacBio sequencer. In some embodiments, (f) comprises a sequencing by ligation approach. In some embodiments, (f) comprises a nanopore based sequencing approach. In some embodiments, (f) comprises a sequence hybridization approach. In some embodiments, (f) comprises a ligation-based approach.

In another aspect, provided herein is a method comprising: (a) tagging a peptide with a plurality of barcodes comprising different barcode sequences; (b) coupling a dual primer linker sequence to two adjacent barcode sequences of the plurality of barcodes; (c) adding a polymerase to copy one of the adjacent barcode sequences of the two adjacent barcode sequences to the other adjacent barcode sequence of the two adjacent barcode sequences; and (d) sequencing the peptide.

In some embodiments, (d) comprises: (i) contacting a Barcode Transfer Reagent (BTR) to a terminal amino acid or a terminal amino acid derivative of the peptide, wherein the BTR and the terminal amino acid or the terminal amino acid derivative generate a barcoded-amino acid complex (BTR-AC) comprising barcode information; (ii) cleaving the BTR-AC from the peptide to release the BTR-AC; (iii) contacting the BTR-ACs with a binding agent; (iv) sorting the BTR-AC into groups; and (v) reading out the barcode information from the BTR-AC. In some embodiments, the method further comprises prior to (d), fragmenting the peptide. In some embodiments, the method further comprises, sequencing the dual primer linker sequence or derivative thereof, thereby identifying the two adjacent barcode sequences as arising from the peptide.

In some embodiments, the BTR or a barcode of the plurality of barcodes further comprises a hairpin segment. In some embodiments, the BTR or a barcode of the plurality of barcodes comprises one or more artificial nucleic acids. In some embodiments, the one or more artificial nucleic acids are locked-nucleic acids (LNA). In some embodiments, the one or more artificial nucleic acids are peptide nucleic acids (PNA). In some embodiments, the BTR or a barcode of the plurality of barcodes comprises a peptide. In some embodiments, the BTR or a barcode of the plurality of barcodes comprises a chemical polymer. In some embodiments, the BTR or a barcode of the plurality of barcodes comprises a heavy metal tag. In some embodiments, the BTR or a barcode of the plurality of barcodes further comprises a primer binding site.

In some embodiments, the method further comprises attaching the dual primer linker sequence to the primer binding site. the method further comprises copying the adjacent barcode to the barcode conjugated to the peptide via the polymerase.

In some embodiments, the binding agent comprises an antibody. In some embodiments, the binding agent comprises a nanobody. In some embodiments, the binding agent comprises a modified amino acyl tRNA transferase. In some embodiments, the binding agent comprises an artificial protein domain. In some embodiments, the binding agent comprises an aptamer. In some embodiments, the binding agent comprises an aminopeptidase or a carboxypeptidase. In some embodiments, the binding agent comprises a modified endoprotease. In some embodiments, the binding agent recognizes an individual amino acid. In some embodiments, the binding agent recognizes a specific dipeptide. In some embodiments, the binding agent recognizes a specific tripeptide. In some embodiments, the binding agent recognizes a post-translational modification (PTM). In some embodiments, the binding agent has a barcode associated with a specific amino acid. In some embodiments, (d) comprises sequencing the different barcode sequences or derivative thereof. In some embodiments, the method further comprises sequencing with a Next Generation Sequencing (NGS) platform. In some embodiments, the method further comprises amplifying the dual primer linker sequence. In some embodiments, the method further comprises amplifying the two adjacent barcode sequences.

In some embodiments, (h) comprises a sequencing by synthesis approach. In some embodiments, the sequencing by synthesis approach comprises using an Illumina Sequencer or a PacBio sequencer. In some embodiments, (h) comprises a sequencing by ligation approach. In some embodiments, (h) comprises a nanopore based sequencing approach. In some embodiments, (h) comprises a sequence hybridization approach. In some embodiments, (h) comprises a ligation-based approach.

In another aspect of the present disclosure, provided herein is a method comprising: (a) tagging a native folded protein with a plurality of barcodes to generate a tagged protein; (b) fragmenting the tagged protein into a plurality of peptides; (c) contacting a Barcode Transfer Reagent (BTR) to a terminal amino acid or a terminal amino acid derivative of a peptide of the plurality of peptides, wherein the BTR and the terminal amino acid or the terminal amino acid derivative generate a barcoded-amino acid complex (BTR-AC) comprising barcode information; (d) cleaving the BTR-AC from the peptide to release the BTR-AC; (e) contacting the BTR-AC with a binding agent; (f) repeating steps (c) to (d) at least once to generate a plurality of the BTR-ACs; and (g) reading out the barcode information from the BTR-ACs, thereby identifying which amino acids were exposed on the surface of the protein.

In some embodiments, the plurality of barcodes is attached to surface-exposed amino acids. In some embodiments, the plurality of barcodes comprises DNA or RNA. In some embodiments, the plurality of barcodes further comprises a hairpin segment. In some embodiments, the plurality of barcodes comprises one or more artificial nucleic acids. In some embodiments, the one or more artificial nucleic acids are locked-nucleic acids (LNA). In some embodiments, the one or more artificial nucleic acids are peptide nucleic acids (PNA). In some embodiments, the plurality of barcodes comprises a peptide. In some embodiments, the plurality of barcodes comprises a chemical polymer. In some embodiments, the plurality of barcodes comprises a heavy metal tag.

In some embodiments, fragmenting comprises use of a protease digestion, a chemical treatment, or sonication.

In some embodiments, the binding agent comprises an antibody. In some embodiments, the binding agent comprises a nanobody. In some embodiments, the binding agent is a modified amino acyl tRNA transferase. In some embodiments, the binding agent comprises an artificial protein domain. In some embodiments, the binding agent comprises an aptamer. In some embodiments, the binding agent comprises an aminopeptidase or a carboxypeptidase. In some embodiments, the binding agent comprises a modified endoprotease. In some embodiments, the binding agent recognizes an individual amino acid. In some embodiments, the binding agent recognizes a specific dipeptide. In some embodiments, the binding agent recognizes a specific tripeptide. In some embodiments, the binding agent recognizes a post-translational modification (PTM). In some embodiments, the binding agent comprises a barcode associated with a specific amino acid. In some embodiments, the binding agent comprises a barcode associated with a specific post-translational modification.

In some embodiments, the method further comprises sequencing the barcode information. In some embodiments, sequencing the barcode information with Next Generation Sequencing (NGS) platform. In some embodiments, the method further comprises amplifying the sample. In some embodiments, the method further comprises sequencing the barcode associated with the terminal amino acid or the terminal amino acid derivative of the peptide. In some embodiments, (g) comprises a sequencing by synthesis approach. In some embodiments, the sequencing by synthesis approach comprises using an Illumina Sequencer or a PacBio sequencer. In some embodiments, (g) comprises a sequencing by ligation approach. In some embodiments, (g) comprises a nanopore based sequencing approach. In some embodiments, (g) comprises a sequence hybridization approach. In some embodiments, (g) comprises a ligation-based approach.

In yet another aspect, disclosed herein is a method comprising: (a) performing a functional assay of a library comprising one or more peptides or proteins to identify peptides or proteins of interest; (b) separating the peptides or proteins of interest to generate substantially isolated peptides or proteins; (c) tagging the substantially isolated peptides or proteins with protein-specific barcodes; (d) contacting a Barcode Transfer Reagent (BTR) to a terminal amino acid or a terminal amino acid derivative of a peptide or protein of the substantially isolated peptides or proteins, wherein the BTR and the terminal amino acid or the terminal amino acid derivative generate a barcoded-amino acid complex (BTR-AC) comprising barcode information; (e) cleaving the BTR-AC from the peptide or protein to release the BTR-AC; (f) repeating steps (c) to (e) at least once to generate a plurality of the BTR-ACs; (e) contacting the plurality of BTR-ACs with a binding agent; (g) sorting the plurality of BTR-ACs into groups; and (h) reading out barcode information from the BTR-ACs, thereby sequencing the terminal amino acids.

In some embodiments, the method further comprises performing mutagenesis on the peptides or proteins of interest.

In another aspect, provided herein is a method comprising: (a) generating a library of proteins from a single encoding DNA or RNA sequence by introducing substitutions during translation, thereby performing mutagenesis; (b) tagging one or more proteins from the library of proteins with a barcode; (c) contacting a Barcode Transfer Reagent (BTR) to a terminal amino acid or a terminal amino acid derivative of a protein from the one or more proteins, wherein the BTR and the terminal amino acid or the terminal amino acid derivative generate a barcoded-amino acid complex (BTR-AC) comprising barcode information; (d) cleaving the BTR-AC from the protein to release the BTR-AC; (e) repeating steps (b) to (d) at least once to generate a plurality of the BTR-ACs; (f) contacting the plurality of BTR-ACs with a binding agent; (g) sorting the plurality of BTR-ACs into groups; and (h) reading out barcode information from the BTR-ACs, thereby sequencing the one or more proteins.

In some embodiments, performing mutagenesis comprises introducing one or more tRNA molecules, wherein the one or more tRNA molecules are charged with different or missense amino acids. In some embodiments, performing mutagenesis comprises altering the conditions of prokaryotic or eukaryotic based ribosome translation to introduce errors.

In another aspect, provided herein is a method of generating a molecular target profile comprising: (a) mixing a molecule with a first protein target to form a complex, and exposing the complex to a protease to generate one or more fragments of the complex; (b) exposing a second protein target to a protease to generate one or more fragments of a protein target; (c) labeling the one or more fragments of the protein target and the one or more fragments of the complex with a barcode to generate one or more barcoded fragments; and (d) sequencing the one or more barcoded fragments, wherein sequencing the one or more fragments comprises: (i) contacting a Barcode Transfer Reagent (BTR) to a terminal amino acid or a terminal amino acid derivative of the one or more barcoded fragments, wherein the BTR and the terminal amino acid or the terminal amino acid derivative generate a barcoded-amino acid complex (BTR-AC); (ii) cleaving the BTR-AC from the one or more barcoded fragments to release the BTR-AC; (iii) repeating steps (i) to (iii) at least once to generate a plurality of the BTR-ACs; (iv) contacting the plurality of BTR-ACs with one or more of an binding agent; (v) sorting the plurality of BTR-ACs into groups; and (vi) reading out barcode information from the BTR-ACs, thereby sequencing the barcoded fragments; and (e) evaluating one or more features of the one or more fragments of the complex and one or more features of the one or more fragments of the protein target, thereby generating a molecular target profile.

In another aspect, provided herein is a method for preparing a multimeric binding agent, the method comprising linking or fusing one or more binding agents, thereby preparing the multimeric binding agent.

In another aspect, disclosed herein is a method of conjugating a chemical tag to a peptide or protein, the method comprising tagging a peptide or protein with a chemical tag; wherein the chemical tag is attached to an enzyme substrate; and wherein the chemical tag conjugates the peptide or protein with a surface.

In some embodiments, the chemical tag is reactive and covalently conjugates to the surface. In some embodiments, the chemical tag is enzymatically conjugated to the surface.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

To better understand various example embodiments, reference is made to the accompanying drawings, wherein:

FIG. 1 provides a diagram showing the series of operations for sequencing a peptide using nucleic acid barcode molecules.

FIG. 2 provides an illustration of an embodiment of the process described herein, where barcode round or cycle information is present on the Barcoded Transfer Reagent. In the embodiment shown, polymerase extension to record the interaction between an amino-acid specific binding agent and a BTR-amino acid complex (BTR-AC). Upon the binding of a BTR-AC molecule to the binding agent, a primer associated with the binding agent hybridizes to a complementary region on the BTR-AC. This primer is extended with a polymerase to copy the information of the BTR-AC. Afterwards, the binding agent and the BTR-AC are dissociated. This process can be iteratively repeated to generate many reads, which improves the accuracy of the detection.

FIG. 3A and FIG. 3B show an overview of example processes provided herein, with FIG. 3A providing a higher-level schematic of the process.

FIG. 4 illustrates an embodiment of the present disclosure, wherein the Barcoded Transfer Reagent is added and conjugates to the terminal end of the protein. In this embodiment, sequencing is performed from the N-terminus using DNA barcodes.

FIG. 5 provides a scheme for transfer of barcode information from the BTR reagent to the protein barcode using ligation and polymerase extension. Once a BTR, containing its own unique barcode, reacts to a terminal amino acid, a ligase is used to attach the free end of the BTR to the free end of the protein barcode. Then, a polymerase extension step creates a double stranded segment between the two ligated regions. Finally, cleavage of the double stranded segment with a restriction enzyme releases the protein barcode with a copy of the BTR barcode sequence. The BTR along with the terminal amino acid is cleaved and the whole process is repeated for the new terminal amino acid. Figure discloses SEQ ID NO: 10.

FIG. 6A provides a schematic for example sample processing operations with alternative options. FIG. 6B provides a schematic detailing options for the protein sequencing chemistry portion of the workflow. FIG. 6C provides a schematic showing iterations and example options for the identification of cleaved amino acids in the workflow.

FIG. 7 provides sample processing operations for single-cell experiments. Different types of biological samples can be processed to yield cell suspensions.

FIG. 8 provides a generalized workflow for processing single-cell samples beginning with cell suspensions and resulting in proteins with cell-specific barcodes.

FIG. 9 provides a single cell-specific sample preparation for protein sequencing, which involves sorting individual cells into wells and using barcoded beads to tag and tether proteins.

FIG. 10 provides single cell-specific sample preparation for protein sequencing, which involves encapsulating single cells in water-oil emulsion droplets along with barcoded beads that tag and tether proteins.

FIG. 11 provides a generalized sample processing workflow for generating proteins bearing spatial barcodes from intact specimens such as tissue samples.

FIG. 12 illustrates preservation of spatial location of proteins using a spatially organized 2D bead array. Intact samples are placed on the bead array and proteins are transferred from the sample to the beads via a protein anchoring reagent, which attaches proteins to spatial barcodes on beads. These protein bearing barcoded beads are then collected for downstream processing.

FIG. 13 depicts an example of tagging proteins in samples with spatial barcodes using a 3D hydrogel. Intact samples are embedded in a 3D hydrogel, and proteins from samples are transferred to the hydrogel via a gel anchoring reagent. Once transferred, deterministic methods such as two-photon patterning or stochastic methods such as PCR, are used to assign different segments of the 3D hydrogel with unique spatial barcodes. Finally, proteins now bearing barcodes, which denote their original 3D locations, are detached from the gel and collected for downstream processing.

FIG. 14 shows peptides such as those generated from fragmented proteins can be tagged with barcodes at the C- or N-termini, or on internal residues.

FIG. 15 provides example chemistries that can be used to attach barcodes to proteins.

FIG. 16 provides example post-translational modifications (PTMs), such as phosphorylation sites, which can be processed into stable moieties that can be recognized with binding agents.

FIG. 17A-17C provide abstract workflows for transferring barcode information between barcoded proteins and the Barcode Transfer Reagent (BTR).

FIG. 18A provides a diagram showing an approach for transferring amino acid specific information from the BTR to the protein barcode. Each BTR contains a unique UMI sequence. The UMI sequence is transferred to the protein barcode during each round or cycle via ligation and cleavage steps, yielding a recording tag with the UMI sequences from each step. These UMI sequences can then be associated with their respective amino acids when the cleaved BTRs are identified and read out via NGS sequencing. FIG. 18B provides an example protein barcode recording tag output that remains after dissociation of the binding agent and BTR-AC.

FIG. 19 provides a diagram showing an approach for using amplification to record the interaction between a BTR and a protein barcode. When a BTR binds to the terminal amino acid of a barcoded protein, the barcode of the BTR is ligated to the barcode of the protein. Amplification of both the BTR and protein barcode regions preserves a copy of the interaction to be used downstream in reconstructing protein sequences.

FIG. 20A-20C provide abstract workflows for recording the interactions between a cleaved BTR with a terminal amino acid (BTR-AC) and barcode-bearing amino acid-specific binding agents.

FIG. 21 illustrates an embodiment for recording the interaction of a BTR-AC and a barcoded amino acid-binding agent using ligation and cleavage steps. Upon the binding of a BTR-AC to the binding agent, the barcode of the binding agent is coupled to the barcode of the BTR-AC via a linker (e.g., splint molecule). These coupled barcodes are then released from the binding agent, retaining a record of the interaction in the form of the coupled sequences.

FIG. 22 demonstrates an embodiment using polymerase extension to record the interaction between an amino acid-specific binding agent and a BTR-AC. Upon the binding of a BTR-AC molecule to the binding agent, a primer associated with the binding agent hybridizes to a complementary region on the BTR-AC. This primer is extended to copy the information of the BTR-AC. Afterwards, the binding agent and the BTR-AC are dissociated.

FIG. 23 illustrates an embodiment comprising recording the interaction between BTR-ACs and amino acid-specific binding agents using PCR, followed by iteratively repeating the process. Upon the binding of a BTR-AC to a barcoded binding agent, their respective barcodes are ligated, and the ligated product is amplified via PCR as a record of the interaction. Subsequently, the ligated product is cleaved using an endonuclease. This process can be iteratively repeated to improve the accuracy of detection.

FIG. 24 illustrates an embodiment comprising labeling a protein on multiple sites with copies of a single barcode using bridge amplification.

FIG. 25 provides a scheme for recording the interaction between a BTR and a barcoded protein as well as the identity of the terminal amino acid. Upon the binding of a BTR to the terminal amino acid of a protein, their respective barcodes are ligated, and the terminal amino acid is cleaved, generating a peptide-peptide barcode-cleaved terminal amino acid complex. The complex is exposed to barcoded amino acid-specific binding agents which recognize and bind to the cleaved terminal amino acid. Primers on the binding agents are used to copy, via an extension reaction, the sequences of the BTR and protein barcode.

FIG. 26 shows example data showing transfer or recording of a peptide barcode to a BTR barcode.

FIG. 27 shows example data showing cleavage of a BTR-AC from a peptide.

FIG. 28 shows example data of a pulldown assay using bead-based binding agents.

FIG. 29 schematically shows a computer system described herein.

### DETAILED DESCRIPTION

To facilitate an understanding of the principles and features of the various embodiments of the disclosure, various illustrative embodiments are explained below. Although example embodiments of the disclosure are explained in detail, it is to be understood that other embodiments are contemplated. Accordingly, it is not intended that the disclosure is limited in its scope to the details of construction and arrangement of components set forth in the following description or examples. The disclosure is capable of other embodiments and of being practiced or carried out in various ways.

Also, in describing the example embodiments, specific terminology will be resorted to for the sake of clarity. It is intended that each term contemplates its broadest meaning as understood by those skilled in the art and includes all technical equivalents which operate in a similar manner to accomplish a similar purpose. It is to be understood that embodiments of the disclosed technology may be practiced without these specific details. In other instances, well-known methods, structures, and techniques have not been shown in detail in order not to obscure an understanding of this description.

### Definitions

References to “one embodiment,” “an embodiment,” “example embodiment,” “some embodiments,” “certain embodiments,” “various embodiments,” etc., indicate that the embodiment(s) of the disclosed technology so described may include a particular feature, structure, or characteristic, but not every embodiment necessarily includes the particular feature, structure, or characteristic. Further, repeated use of the phrase “in one embodiment” does not necessarily refer to the same embodiment, although it may.

Ranges may be expressed herein as from “about” or “approximately” or “substantially” one particular value and/or to “about” or “approximately” or “substantially” another particular value. When such a range is expressed, other example embodiments include from the one particular value and/or to the other particular value. Further, the term “about” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to +20%, preferably up to +10%, more preferably up to ±5%, and more preferably still up to +1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

Throughout this description, various components may be identified having specific values or parameters, however, these items are provided as example embodiments. Indeed, the example embodiments do not limit the various aspects and concepts of the present disclosure as many comparable parameters, sizes, ranges, and/or values may be implemented. The terms “first,” “second,” and the like, “primary,” “secondary,” and the like, do not denote any order, quantity, or importance, but rather are used to distinguish one element from another.

As used herein, the term “protein” generally refers to a molecule comprising two or more amino acids joined by a peptide bond. A protein may also be referred to as a “polypeptide”, “oligopeptide”, or “peptide”. A protein can be a naturally occurring molecule, or a synthetic molecule. A protein may include one or more non-natural amino acids, modified amino acids, or non-amino acid linkers. A protein may contain D-amino acid enantiomers, L-amino acid enantiomers or both. Amino acids of a protein may be modified naturally or synthetically, such as by post-translational modifications. In some circumstances, different proteins may be distinguished from each other based on different genes from which they are expressed in an organism, different primary sequence length or different primary sequence composition. Proteins expressed from the same gene may nonetheless be different proteoforms, for example, being distinguished based on non-identical length, non-identical amino acid sequence or non-identical post-translational modifications. Different proteins can be distinguished based on one or both of gene of origin and proteoform state.

As used herein, the term “peptide” may generally refer to any short, single peptide chain. A peptide may be no more than about 100, 95, 90, 85, 80, 75, 70, 65, 60, 55, 50, 45, 40, 35, 30, 25, 20, 15, 10, 5, or less than about 5 amino acids in length. A peptide may have a known or unknown biological function or activity. Peptides can include natural, synthetic, modified, or degraded proteins or peptides.

As used herein, the term “single analyte” generally refers to an analyte (e.g., protein, nucleic acid, or affinity reagent) that is individually manipulated or distinguished from other analytes. A single analyte can be a single molecule (e.g., single protein), a single complex of two or more molecules (e.g., a multimeric protein having two or more separable subunits, a single protein attached to a structured nucleic acid particle or a single protein attached to an affinity reagent), a single particle, or the like. Reference herein to a “single analyte” in the context of a composition, system or method herein does not necessarily exclude application of the composition, system or method to multiple single analytes that are manipulated or distinguished individually, unless indicated contextually or explicitly to the contrary.

As used herein, “polypeptide” generally refers to two or more amino acids linked together by a peptide bond. The term “polypeptide” includes proteins that have a C-terminal end and an N-terminal end as generally known in the art and may be synthetic in origin or naturally occurring. As used herein “at least a portion of the polypeptide” refers to 2 or more amino acids of the polypeptide. Optionally, a portion of the polypeptide includes at least: 5, 10, 20, 30 or 50 amino acids, either consecutive or with gaps, of the complete amino acid sequence of the polypeptide, or the full amino acid sequence of the polypeptide.

As used herein, “affixed” generally refers to a connection between a polypeptide and a substrate such that at least a portion of the polypeptide and the substrate are held in physical proximity. The term “affixed” encompasses both an indirect or direct connection and may be reversible or irreversible, for example the connection is optionally a covalent bond or a non-covalent bond.

As used herein, the term “sample” generally refers to a collected substance or material that comprises or is suspected to comprise one or more analytes of interest (e.g., polypeptides). A sample may be modified for purposes such as storage or stability. A sample may have undergone one or more processes that separate or remove unwanted fractions or impurities from the analyte(s) of interest. For example, a fraction is a type of sample. Alternatively, a sample may not have undergone any processes that separates or removes any unwanted fractions or impurities from the analyte(s) of interest. For example, a fluid, tissue, or cell is a type of sample. A sample may include biological and/or non-biological components. As used herein, the terms “biological sample” or “biological source” refer to a sample that is derived from a predominantly biological system or organism, such as one or more viral particles, cells (e.g. individualized cells), organelles (e.g. individualized organelles), tissues, bodily fluids, bone, cartilage, and exoskeleton. A biological sample may comprise a majority of biological material on a mass basis, excluding the weight of fluid within the sample. Biological samples may comprise proteins, referred to herein as protein samples. Protein samples can be acquired from various sources as needed. For example, protein samples might be derived from clinical patient samples, such as blood, Cerebral Spinal Fluid (CSF), or saliva, in which case these samples will be processed to purify and retain proteins. Alternatively, protein samples can result from cellular and single cell samples. For example, samples may be derived from cultures of induced pluripotent stem cells (iPSCs). Samples may also be drug-treated samples of cultured mammalian cells. Proteins would then be extracted from such cell samples. Protein samples can also result from tissue specimens, such as biopsy samples, in which case such tissues would need to be processed as needed to liberate the proteins they contain. Tissue samples may also be derived from in vivo specimens, including Fresh Frozen, acute, and fixed. Finally, protein and peptide samples might also be acquired from environmental specimens, such as water samples or food samples.

The terms “antibody” and “immunoglobulin” include antibodies or immunoglobulins of any isotype, fragments of antibodies which retain specific binding to antigen, including, but not limited to, Fab, Fv, scFv, and Fd fragments, chimeric antibodies, humanized antibodies, single-chain antibodies, and fusion proteins including an antigen-binding portion of an antibody and a non-antibody protein. The antibodies may be detectably labeled, e.g., with a radioisotope, a heavy metal tag, a mass tag, an enzyme which generates a detectable product, a fluorescent protein, a nucleic acid barcode sequence, and the like. The antibodies may be further conjugated to other moieties, such as members of specific binding pairs, e.g., biotin (member of biotin-avidin specific binding pair), and the like. Also encompassed by the terms are Fab′, Fv, F(ab′)2, and other antibody fragments that retain specific binding to antigen. Antibodies may exist in a variety of other forms including, for example, Fv, Fab, and (Fab)2, as well as bi-functional (i.e., bi-specific) hybrid antibodies (e.g., Lanzavecchia et al., Eur. J. Immunol. 17, 105 (1987)) and in single chains (e.g., Huston et al., Proc. Natl. Acad. Sci. U.S.A., 85, 5879-5883 (1988) and Bird et al., Science, 242, 423-426 (1988), which are incorporated herein by reference). (See, generally, Hood et al., Immunology, Benjamin, N.Y., 2nd ed. (1984), and Hunkapiller and Hood, Nature, 323, 15-16 (1986).

“Binding” as used herein generally refers to a covalent or non-covalent interaction between two molecules (referred to herein as “binding partners”, e.g., a substrate and an enzyme or an antibody and an epitope), which binding is usually specific.

As used herein, “specifically binds” or “binds specifically” generally refers to interaction between binding partners such that the binding partners bind to one another, but do not bind other molecules that may be present in the environment (e.g., in a biological sample, in tissue) at a significant or substantial level under a given set of conditions (e.g., physiological conditions).

The terms “DNA”, “nucleic acid”, “nucleic acid molecule”, “oligonucleotide” and “polynucleotide” are used interchangeably and generally refer to a polymeric form of naturally occurring or synthetic nucleotides of any length, either deoxyribonucleotides or ribonucleotides, hexitol nucleotides, cyclohexane nucleotides, or analogs thereof. The terms encompass, e.g., DNA, RNA, HNA, CeNA, and modified forms thereof. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, control regions, isolated RNA of any sequence, nucleic acid probes, and primers. The nucleic acid molecule may be linear or circular.

As used herein, the term “amino acid” generally refers to an organic compound comprising an amine group, a carboxylic acid group, and a side-chain specific to each amino acid, which serve as a monomeric subunit of a peptide. An amino acid includes the 20 standard, naturally occurring or canonical amino acids as well as non-standard amino acids. The standard, naturally-occurring amino acids include Alanine (A or Ala), Cysteine (C or Cys), Aspartic Acid (D or Asp), Glutamic Acid (E or Glu), Phenylalanine (F or Phe), Glycine (G or Gly), Histidine (H or His), Isoleucine (I or Ile), Lysine (K or Lys), Leucine (L or Leu), Methionine (M or Met), Asparagine (N or Asn), Proline (P or Pro), Glutamine (Q or Gln), Arginine (R or Arg), Serine (S or Ser), Threonine (T or Thr), Valine (V or Val), Tryptophan (W or Trp), and Tyrosine (Y or Tyr). An amino acid may be an L-amino acid or a D-amino acid. Non-standard amino acids may be modified amino acids, amino acid analogs, amino acid mimetics, non-standard proteinogenic amino acids, or non-proteinogenic amino acids that occur naturally or are chemically synthesized. Examples of non-standard amino acids include, but are not limited to, selenocysteine, pyrrolysine, and N-formylmethionine, (3-amino acids, Homo-amino acids, Proline and Pyruvic acid derivatives, 3-substituted alanine derivatives, glycine derivatives, ring-substituted phenylalanine and tyrosine derivatives, linear core amino acids, N-methyl amino acids.

As used herein, the term “post-translational modification” generally refers to modifications that occur on a peptide after its translation by ribosomes is complete. A post-translational modification may be a covalent modification or enzymatic modification. Examples of post-translation modifications include, but are not limited to, acylation, acetylation, alkylation (including methylation), biotinylation, butyrylation, carbamylation, carbonylation, deamidation, deiminiation, diphthamide formation, disulfide bridge formation, eliminylation, flavin attachment, formylation, gamma-carboxylation, glutamylation, glycylation, glycosylation, glypiation, heme C attachment, hydroxylation, hypusine formation, iodination, isoprenylation, lipidation, lipoylation, malonylation, methylation, myristolylation, oxidation, palmitoylation, pegylation, phosphopantetheinylation, phosphorylation, prenylation, propionylation, retinylidene Schiff base formation, S-glutathionylation, S-nitrosylation, S-sulfenylation, selenation, succinylation, sulfination, ubiquitination, and C-terminal amidation. A post-translational modification includes modifications of the amino terminus and/or the carboxyl terminus of a peptide. Modifications of the terminal amino group include, but are not limited to, des-amino, N-lower alkyl, N-di-lower alkyl, and N-acyl modifications. Modifications of the terminal carboxy group include, but are not limited to, amide, lower alkyl amide, dialkyl amide, and lower alkyl ester modifications (e.g., wherein lower alkyl is C1-C4 alkyl). A post-translational modification also includes modifications, such as but not limited to those described above, of amino acids falling between the amino and carboxy termini. The term post-translational modification can also include peptide modifications that include one or more detectable labels.

As used herein, the term “binding agent” generally refers to a molecule that recognizes, binds to, associates with, unites with, recognizes, or combines with another molecule. The binding agent may comprise a nucleic acid molecule, a peptide, a polypeptide, a protein, carbohydrate, a synthetic macromolecule, or a small molecule that binds to, associates, unites with, recognizes, or combines with a molecule, macromolecule, or a component or feature of a molecule or macromolecule. A binding agent may form a covalent association or non-covalent association with the molecule or macromolecule or component or feature of a molecule or macromolecule. A binding agent may also be a chimeric binding agent, composed of two or more types of molecules, such as a nucleic acid molecule-peptide chimeric binding agent or a carbohydrate-peptide chimeric binding agent. A binding agent may be a naturally occurring, synthetically produced, or recombinantly expressed molecule. A binding agent may bind to a single monomer or subunit of a molecule or macromolecule (e.g., a single amino acid of a peptide) or bind to a plurality of linked subunits of a molecule or macromolecule (e.g., a di-peptide, tri-peptide, or higher order peptide of a longer peptide, polypeptide, or protein molecule). A binding agent may bind to a linear molecule or a molecule having a three-dimensional structure (also referred to as conformation). For example, an antibody binding agent may bind to linear peptide, polypeptide, or protein, or bind to a conformational peptide, polypeptide, or protein. A binding agent may bind to an N-terminal peptide, a C-terminal peptide, or an intervening peptide of a peptide, polypeptide, or protein molecule. A binding agent may bind to an N-terminal amino acid, C-terminal amino acid, or an intervening amino acid of a peptide molecule. A binding agent may preferentially bind to a chemically modified or labeled amino acid over a non-modified or unlabeled amino acid. For example, a binding agent may preferentially bind to an amino acid that has been modified with an acetyl moiety, guanyl moiety, dansyl moiety, phenyl thiocyanate (PTC) moiety, 2,4-dinitrophenol (DNP) moiety, SNP moiety, etc., over an amino acid that does not possess said moiety. A binding agent may bind to a post-translational modification of a peptide molecule. A binding agent may exhibit selective binding to a component or feature of a macromolecule (e.g., a binding agent may selectively bind to one of the 20 possible natural amino acid residues and with bind with very low affinity or not at all to the other 19 natural amino acid residues). A binding agent may exhibit less selective binding, where the binding agent is capable of binding a plurality of components or features of a macromolecule (e.g., a binding agent may bind with similar affinity to two or more different amino acid residues). A binding agent may comprise a coding tag, which may be joined to the binding agent by a linker.

As used herein, the term “linker” generally refers to a molecule or moiety that is involved in joining two or more molecules. A linker may facilitate a covalent or noncovalent interaction of two or more molecules. A linker may be a crosslinker. The linker can be unifunctional, bifunctional, trifunctional, quadrifunctional, or polyfunctional. A linker can be or comprise a nucleotide, a nucleotide analog, an amino acid, a peptide, a polypeptide, or a non-nucleotide chemical moiety, such as an organic or inorganic compound. A linker may comprise a polymer, such as a polyethylene glycol (PEG), poly-L-lysine (PLL), poly (DL-lactic acid) (PLA), poly (DL-lactide-co-glycoside) (PLGA), polyomithine, polyarginine, etc. A linker may comprise one or more reactive ends, e.g., an amine-reactive group, a carboxyl-reactive group, a sulfhydryl-reactive group, a hydroxyl-reactive group, etc. In some examples, a linker may be used to join different molecule types, e.g., different biomolecule types such as a peptide with a nucleic acid molecule, a lipid with a peptide, a carbohydrate with a peptide, etc.; non-biomolecule types; or a biomolecule to a non-biomolecule. For example, a linker may be used to join a binding agent with a tag, a tag with a macromolecule (e.g., peptide, nucleic acid molecule), a macromolecule with a solid support, a tag with a solid support, etc. A linker may join two molecules via enzymatic reaction or chemistry reaction. A linker may comprise one or more click chemistry moieties. A linker may join more than two molecules, e.g., via enzymatic or chemical reactions.

As used herein, the term “proteomics” generally refers to quantitative analysis of the proteome within cells, tissues, and bodily fluids, and the corresponding spatial distribution of the proteome within the cell and within tissues. Additionally, proteomics studies include the dynamic state of the proteome, continually changing in time as a function of biology and defined biological or chemical stimuli.

The terminal amino acid at one end of the peptide chain that has a free amino group is referred to herein as the “N-terminal amino acid” (NTAA). The terminal amino acid at the other end of the chain that has a free carboxyl group is referred to herein as the “C-terminal amino acid” (CTAA). The amino acids making up a peptide may be numbered in order, with the peptide being “n” amino acids in length. As used herein, NTAA is considered the nth amino acid (also referred to herein as the “n NTAA”). Using this nomenclature, the next amino acid is the n-1 amino acid, then the n-2 amino acid, and so on down the length of the peptide from the N-terminal end to C-terminal end. In certain embodiments, an NTAA, CTAA, or both may be modified or labeled with a chemical moiety.

As used herein, the term “barcode” generally refers to an identifying feature that may be used to distinguish similar items. A barcode may comprise a nucleic acid molecule of about 2 to about 30 bases (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, or 150 bases), which may provide a unique identifier tag or origin information for a molecule (e.g., protein, polypeptide, peptide), a binding agent, a set of binding agents from a binding cycle, a sample molecule, a set of samples, molecules within a compartment (e.g., droplet, bead, partition or separated location), macromolecules within a set of compartments, a fraction of macromolecules, a set of macromolecule fractions, a spatial region or set of spatial regions, a library of macromolecules, or a library of binding agents. A barcode can be an artificial sequence or a naturally occurring sequence including peptides, proteins, protein complexes, carbohydrates, and synthetic polymeric materials. In certain embodiments, each barcode within a population of barcodes is different. In other embodiments, a portion of barcodes in a population of barcodes is different, e.g., at least about 10%, 15%, 20%, 25%, 30%, 35%, 40%, 45%, 50%, 55%, 60%, 65%, 70%, 75%, 80%, 85%, 90%, 95%, 97%, or 99% of the barcodes in a population of barcodes is different. A population of barcodes may be randomly generated or non-randomly generated. A population of barcodes may comprise error correcting barcodes. Barcodes can be used to computationally deconvolute sequence reads derived from an individual molecule, sample, library, etc. Barcodes may comprise multiplexed information, e.g., arising from different samples, compartments, individual molecules, etc. A barcode can also be used for deconvolution of a collection of molecules that have been distributed into small compartments for enhanced mapping. For example, rather than mapping a peptide back to the proteome, the peptide can be mapped back to its originating protein molecule or protein complex.

A barcode may comprise any useful structure moiety or motif, e.g., hairpins, loop sequences, or spacers. Barcodes can comprise artificial or modified nucleic acids, e.g., locked nucleic acids (LNA), protein nucleic acids (PNA), hexitol nucleic acids (HNA), cyclohexane nucleic acids (CeNA), or a combination thereof. Barcodes may comprise or be generated using a protein, e.g., Tal effector, Cas protein (e.g., Cas9), Argonaut, or coiled coils.

As used herein, a “sample barcode”, also referred to as “sample tag” generally refers to a barcode molecule comprising identifying information of a sample from which a barcoded molecule derives.

As used herein, a “spatial barcode” generally refers to a barcode molecule comprising identifying information of a region of a 2-D or 3-D sample (e.g., a tissue section) from which a molecule originates or is derived. Spatial barcodes may be used for molecular pathology on tissue sections. A spatial barcode may allow for multiplex sequencing of a plurality of samples or libraries from tissue section(s).

As used herein, a “temporal barcode” generally refers to a barcode molecule comprising time-based information relating to the barcoded molecule. The types of time-based data encoded in a temporal barcode can include information such as a life-time of a barcoded molecule, a time of collection of a sample, a time or duration since the beginning of an experiment or induction with a stimulus, information on the age of a cell or tissue, a sequence of interactions between molecules, a cycle or round that the barcode is provided, among others. It is possible for different types of barcodes (e.g., spatial, temporal, cell-specific) to be combined in one multiplexed barcode.

The term “nucleic acid sequence” or “oligonucleotide sequence” generally refers to a contiguous string of nucleotide bases and in particular contexts also refers to the particular placement of nucleotide bases in relation to each other as they appear in an oligonucleotide. Similarly, the term “polypeptide sequence” or “amino acid sequence” refers to a contiguous string of amino acids and in particular contexts also refers to the particular placement of amino acids in relation to each other as they appear in a polypeptide.

The terms “complementary” or “complementarity” generally refer to polynucleotides (i.e., a sequence of nucleotides) related by base-pairing rules. For example, the sequence “5′-AGT-3′,” is complementary to the sequence “5′-ACT-3′”. Complementarity may be “partial,” in which only some of the nucleic acids' bases are matched according to the base pairing rules, or there may be “complete” or “total” complementarity between the nucleic acids. The degree of complementarity between nucleic acid strands can have significant effects on the efficiency and strength of hybridization between nucleic acid strands under defined conditions. This is of particular importance for methods that depend upon binding between nucleic acids.

As used herein, the term “hybridization” is generally used in reference to the pairing of complementary nucleic acids. Hybridization and the strength of hybridization (i.e., the strength of the association between the nucleic acids) is influenced by such factors as the degree of complementary between the nucleic acids, stringency of the conditions involved, and the melting temperature of the formed hybrid. “Hybridization” methods involve the annealing of one nucleic acid to another, complementary nucleic acid, i.e., a nucleic acid having a complementary nucleotide sequence.

Hybridization is carried out in conditions permitting specific hybridization. The length of the complementary sequences and GC content affects the thermal melting point Tm of the hybridization conditions necessary for obtaining specific hybridization of the target site to the target nucleic acid. Hybridization may be carried out under stringent conditions. The phrase “stringent hybridization conditions” refers to conditions under which a probe will hybridize to its target subsequence, typically in a complex mixture of nucleic acid, but to no other sequences at a detectable or significant level. Stringent conditions are sequence-dependent and will be different in different circumstances. Stringent conditions are those in which the salt concentration is less than about 1.0 M sodium ion, such as less than about 0.01 M, including from about 0.001 M to about 1.0 M sodium ion concentration (or other salts) at a pH between about 6 to about 8 and the temperature is in the range of about 20° C. to about 65° C. Stringent conditions may also be achieved with the addition of destabilizing agents, such as but not limited to formamide.

As used herein, the terms “determining,” “measuring,” “cassessing,” and “assaying” are used interchangeably and include both quantitative and qualitative determinations.

As used herein, the term “unique molecular identifier” or “UMI” generally refers to a nucleic acid molecule of about 3 to about 150 bases (3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, or 150 bases) in length providing a unique identifier tag for each macromolecule (e.g., peptide) or binding agent to which the UMI is linked. A macromolecule UMI can be used to computationally deconvolute sequencing data from a plurality of extended recording tags to identify extended recording tags that originated from an individual macromolecule. A binding agent UMI can be used to identify each individual binding agent that binds to a particular macromolecule. For example, a UMI can be used to identify the number of individual binding events for a binding agent specific for a single BTR-amino acid complex that occurs for a particular peptide molecule. It is understood that when UMI and barcode are both referenced in the context of a binding agent or macromolecule, that the barcode refers to identifying information other than the UMI for the individual binding agent or macromolecule (e.g., sample barcode, compartment barcode, binding cycle barcode).

The term “conjugated” as used herein refers to a covalent or ionic interaction between two entities, e.g., molecules, compounds, or combinations thereof.

A first polynucleotide may be “derived from” a second polynucleotide if it has the same or substantially the same nucleotide sequence as a region of the second polynucleotide, its cDNA, complements thereof, or if it displays sequence identity as described above. This term is not meant to require or imply the polynucleotide must be obtained from the origin cited (although such is encompassed), but rather can be made by any suitable method.

A first polypeptide (or peptide) may be “derived from” a second polypeptide (or peptide) if it is (i) encoded by a first polynucleotide derived from a second polynucleotide, or (ii) displays sequence identity to the second polypeptides as described above. This term is not meant to require or imply the polypeptide must be obtained from the origin cited (although such is encompassed), but rather can be made by any suitable method.

In some embodiments, the discrete entities as described herein are droplets. The terms “drop,” “droplet,” and “microdroplet” are used interchangeably herein, to refer to small, generally spherically structures, containing at least a first fluid phase, e.g., an aqueous phase (e.g., water), bounded by a second fluid phase (e.g., oil) which is immiscible with the first fluid phase. In some embodiments, droplets according to the present disclosure may contain a first fluid phase, e.g., oil, bounded by a second immiscible fluid phase, e.g., an aqueous phase fluid (e.g., water). In some embodiments, the second fluid phase will be an immiscible phase carrier fluid. Thus, droplets according to the present disclosure may be provided as aqueous-in-oil emulsions or oil-in-aqueous emulsions. Droplets may be sized and/or shaped as described herein for discrete entities. For example, droplets according to the present disclosure generally range from 1 μm to 1000 μm, inclusive, in diameter. Droplets according to the present disclosure may be used to encapsulate cells, nucleic acids (e.g., DNA), enzymes, reagents, and a variety of other components. The term droplet may be used to refer to a droplet produced in, on, or by a microfluidic device and/or flowed from or applied by a microfluidic device.

As used herein, the term “carrier fluid” generally refers to a fluid configured or selected to contain one or more discrete entities, e.g., droplets, as described herein. A carrier fluid may include one or more substances and may have one or more properties, e.g., viscosity, which allow it to be flowed through a microfluidic device or a portion thereof, such as a delivery orifice. In some embodiments, carrier fluids include, for example: oil or water, and may be in a liquid or gas phase. Suitable carrier fluids are described in greater detail herein.

As used herein, the term “solid support”, “solid surface”, or “solid substrate” or “substrate” generally refers to any solid material, including porous and non-porous materials, to which a macromolecule (e.g., peptide) can be associated directly or indirectly, by any means known in the art, including covalent and non-covalent interactions, or any combination thereof. A solid support may be two-dimensional (e.g., planar surface) or three-dimensional (e.g., gel matrix or bead). A solid support can be any support surface including, but not limited to, a bead, a microbead, an array, a glass surface, a silicon surface, a plastic surface, a filter, a membrane, nylon, a silicon wafer chip, a flow through chip, a flow cell, a biochip including signal transducing electronics, a channel, a microtiter well, an ELISA plate, a spinning interferometry disc, a nitrocellulose membrane, a nitrocellulose-based polymer surface, a polymer matrix, a nanoparticle, or a microsphere. Materials for a solid support include but are not limited to acrylamide, agarose, cellulose, nitrocellulose, glass, gold, quartz, polystyrene, polyethylene vinyl acetate, polypropylene, polymethacrylate, polyethylene, polyethylene oxide, polysilicates, polycarbonates, Teflon, fluorocarbons, nylon, silicon rubber, polyanhydrides, polyglycolic acid, polylactic acid, polyorthoesters, functionalized silane, polypropylfumerate, collagen, gly cosaminogly cans, polyamino acids, dextran, or any combination thereof. Solid supports further include thin film, membrane, bottles, dishes, fibers, woven fibers, shaped polymers such as tubes, particles, beads, microspheres, microparticles, or any combination thereof. For example, when solid surface is a bead, the bead can include, but is not limited to, a ceramic bead, polystyrene bead, a polymer bead, a methylstyrene bead, an agarose bead, an acrylamide bead, a solid core bead, a porous bead, a magnetic or paramagnetic bead, a glass bead, or a controlled pore bead. A bead may be spherical or an irregularly shaped. A bead's size may range from nanometers, e.g., 100 nm, to millimeters, e.g., 1 mm. In certain embodiments, beads range in size from about 0.2 micron to about 200 microns, or from about 0.5 micron to about 5 micron. n some embodiments, beads can be about 1, 1.5, 2, 2.5, 2.8, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μm in diameter. In certain embodiments, “a bead” solid support may refer to an individual bead or a plurality of beads.

As used herein, the term “nucleic acid molecule” or “polynucleotide” generally refers to a single- or double-stranded polynucleotide containing deoxyribonucleotides or ribonucleotides that are linked by 3′-5′ phosphodiester bonds, as well as polynucleotide analogs. A nucleic acid molecule includes, but is not limited to, DNA, RNA, and cDNA. A polynucleotide analog may possess a backbone other than a standard phosphodiester linkage found in natural polynucleotides and, optionally, a modified sugar moiety or moieties other than ribose or deoxyribose. Polynucleotide analogs contain bases capable of hydrogen bonding by Watson-Crick base pairing to standard polynucleotide bases, where the analog backbone presents the bases in a manner to permit such hydrogen bonding in a sequence-specific fashion between the oligonucleotide analog molecule and bases in a standard polynucleotide. Examples of polynucleotide analogs include but are not limited to xeno nucleic acid (XNA), bridged nucleic acid (BNA), glycol nucleic acid (GNA), hexitol nucleic acid (HNA), cyclohexane nucleic acid (CeNA), peptide nucleic acids (PNAs), γPNAs, morpholino polynucleotides, locked nucleic acids (LNAs), threose nucleic acid (TNA), 2′-O-Methyl polynucleotides, 2′-O-alkyl ribosyl substituted polynucleotides, phosphorothioate polynucleotides, and boronophosphate polynucleotides. A polynucleotide analog may possess purine or pyrimidine analogs, including for example, 7-deaza purine analogs, 8-halopurine analogs, 5-halopyrimidine analogs, or universal base analogs that can pair with any base, including hypoxanthine, nitroazoles, isocarbostyril analogues, azole carboxamides, and aromatic triazole analogues, or base analogs with additional functionality, such as a biotin moiety for affinity binding.

As used herein, “nucleic acid sequencing” generally means the determination of the order of nucleotides in a nucleic acid molecule or a sample of nucleic acid molecules.

As used herein, “next generation sequencing” generally refers to high-throughput sequencing methods that allow the sequencing of millions to billions of molecules in parallel. Examples of next generation sequencing methods include sequencing by synthesis, sequencing by ligation, sequencing by hybridization, polony sequencing, ion semiconductor sequencing, nanopore sequencing, and pyrosequencing. By attaching primers to a solid substrate and a complementary sequence to a nucleic acid molecule, a nucleic acid molecule can be hybridized to the solid substrate via the primer and then multiple copies can be generated in a discrete area on the solid substrate by using polymerase to amplify (these groupings are sometimes referred to as polymerase colonies or polonies). Consequently, during the sequencing process, a nucleotide at a particular position can be sequenced multiple times (e.g., hundreds or thousands of times)—this depth of coverage is referred to as “deep sequencing.” Examples of high throughput nucleic acid sequencing technology include platforms provided by Illumina, BGI, Qiagen, ThermoFisher, and Roche, including formats such as parallel bead arrays, sequencing by synthesis, sequencing by ligation, capillary electrophoresis, electronic microchips, “biochips,” microarrays, parallel microchips, and single-molecule arrays, as reviewed by Service (Science 311:1544-1546, 2006).

As used herein, “single molecule sequencing” generally refers to next-generation sequencing methods wherein reads from single molecule sequencing instruments are generated by sequencing of a single molecule, such as a single molecule of a polynucleotide or a polypeptide.

As used herein, “analyzing” the macromolecule generally means to quantify, characterize, distinguish, or a combination thereof, all or a portion of the components of the macromolecule. For example, analyzing a peptide, polypeptide, or protein includes determining all or a portion of the amino acid sequence (contiguous or non-continuous) of the peptide. Analyzing a macromolecule also includes partial identification of a component of the macromolecule. For example, partial identification of amino acids in the macromolecule protein sequence can identify an amino acid in the protein as belonging to a subset of possible amino acids. Analysis typically begins with analysis of the n NTAA, and then proceeds to the next amino acid of the peptide (i.e., n-1, n-2, n-3, and so forth). This is accomplished by cleavage of the n NTAA, thereby converting the n-1 amino acid of the peptide to an N-terminal amino acid (referred to herein as the “n-1 NTAA”). The analysis can also begin from C-terminus towards the N-terminus with each round or cycle of cleavage from the C-terminus creating a new CTAA. Cleavage of the n CTAA converts the n-1 amino acid of the peptide to a C-terminal amino acid, referred to herein as an “n-1 CTAA”. Analyzing the peptide may also include determining the presence and frequency of post-translational modifications on the peptide, which may or may not include information regarding the sequential order of the post-translational modifications on the peptide. Analyzing the peptide may also include determining the presence and frequency of epitopes in the peptide, which may or may not include information regarding the sequential order or location of the epitopes within the peptide. Analyzing the peptide may include combining different types of analysis, for example obtaining epitope information, amino acid sequence information, post-translational modification information, or any combination thereof.

As used herein, the term “compartment” generally refers to a physical area or volume that separates or isolates a subset of macromolecules from a sample of macromolecules. For example, a compartment may separate an individual cell from other cells, or a subset of a sample's proteome from the rest of the sample's proteome. A compartment may be an aqueous compartment (e.g., microfluidic droplet), a solid compartment (e.g., picotiter well or microtiter well on a plate, tube, vial, gel bead), or a separated region on a surface. A compartment may comprise one or more beads to which macromolecules may be immobilized.

As used herein, the term “array” generally refers to a population of molecules that is attached to one or more solid supports such that the molecules at one address can be distinguished from molecules at other addresses. An array can include different molecules that are each located at different addresses on a solid support. Alternatively, an array can include separate solid supports each functioning as an address that bears a different molecule, wherein the different molecules can be identified according to the locations of the solid supports on a surface to which the solid supports are attached, or according to the locations of the solid supports in a liquid such as a fluid stream. The molecules of the array can be, for example, nucleic acids such as SNAP-tagged nucleic acids, polypeptides, proteins, peptides, oligopeptides, enzymes, ligands, or receptors such as antibodies, functional fragments of antibodies or aptamers. The addresses of an array can optionally be optically observable, and, in some configurations, adjacent addresses can be optically distinguishable when detected using a method or apparatus set forth herein.

As used herein, the term “functionalized” generally refers to any material or substance that has been modified to include a functional group. A functionalized material or substance may be naturally or synthetically functionalized. For example, a polypeptide can be naturally functionalized with a phosphate, oligosaccharide (e.g., glycosyl, glycosylphosphatidylinositol or phosphoglycosyl), nitrosyl, methyl, acetyl, lipid (e.g., glycosyl phosphatidylinositol, myristoyl or prenyl), ubiquitin or other naturally occurring post-translational modification. A functionalized material or substance may be functionalized for any given purpose, including altering chemical properties (e.g., altering hydrophobicity or changing surface charge density) or altering reactivity (e.g., capable of reacting with a moiety or reagent to form a covalent bond to the moiety or reagent).

As used herein, the term “anchoring group” generally refers to a molecule or particle that serves as an intermediary attaching a protein or peptide to a surface (e.g., a solid support or a microbead). An anchoring group may be covalently or non-covalently attached to a surface and/or a polypeptide. An anchoring group may be a biomolecule, polymer, particle, nanoparticle, or any other entity that can attach to a surface or polypeptide. In some cases, an anchoring group may be a structured nucleic acid particle.

As used herein, the term “click reaction” or “bioorthogonal reaction” generally refers to single-step, thermodynamically favorable conjugation reaction utilizing biocompatible reagents. A click reaction may utilize no toxic or biologically incompatible reagents (e.g., acids, bases, heavy metals) or generate no toxic or biologically incompatible byproducts. A click reaction may utilize an aqueous solvent or buffer (e.g., phosphate buffer solution, Tris buffer, saline buffer, MOPS, etc.). A click reaction may be thermodynamically favorable if it has a negative Gibbs free energy of reaction, for example a Gibbs free energy of reaction of less than about −5 kiloJoules/mole (kJ/mol), −10 kJ/mol, −25 kJ/mol, −50 kJ/mol, −100 kJ/mol, −200 kJ/mol, −300 kJ/mol, −400 kJ/mol, or less than −500 kJ/mol. Example bioorthogonal and click reactions are described in detail in WO 2019/195633A1, which is herein incorporated by reference in its entirety. Example click reactions may include metal-catalyzed azide-alkyne cycloaddition, strain-promoted azide-alkyne cycloaddition, strain-promoted azide-nitrone cycloaddition, strained alkene reactions, thiolene reaction, Diels-Alder reaction, inverse electron demand Diels-Alder reaction, [3+2] cycloaddition, [4+1] cycloaddition, nucleophilic substitution, dihydroxylation, thiolyne reaction, photoclick, nitrone dipole cycloaddition, norbornene cycloaddition, oxanobomadiene cycloaddition, tetrazine ligation, and tetrazole photoclick reactions. Example functional groups or reactive handles utilized to perform click reactions may include alkenes, alkynes, azides, epoxides, amines, thiols, nitrones, isonitriles, isocyanides, aziridines, activated esters, and tetrazines.

As used herein, the terms “group” and “moiety” are intended to be synonymous when used in reference to the structure of a molecule. The terms refer to a component or part of the molecule. The terms do not necessarily denote the relative size of the component or part compared to the molecule, unless indicated otherwise. The terms do not necessarily denote the relative size of the component or part compared to any other component or part of the molecule, unless indicated otherwise. A group or moiety can contain one or more atom.

A “nucleotide sequence” according to the present invention may include any polymer or oligomer of nucleotides such as pyrimidine and purine bases, preferably cytosine, thymine, and uracil, and adenine and guanine, respectively and combinations thereof. The present invention contemplates any deoxyribonucleotide, ribonucleotide, hexitol-nucleotide, cyclohexane-nucleotide, peptide nucleic acid component, and any chemical variants thereof, such as methylated, 7-deaza purine analogs, 8-halopurine analogs, hydroxymethylated or glycosylated forms of these bases, and the like. The polymers or oligomers may be heterogeneous or homogenous in composition and may be isolated from naturally occurring sources or may be artificially or synthetically produced. In addition, a nucleotide sequence may be DNA, RNA, HNA, CeNA or a mixture thereof, and may exist permanently or transitionally in single-stranded or double-stranded form, including homoduplex, heteroduplex, and hybrid states.

“Amplification” or “amplifying” generally refers to a polynucleotide amplification reaction, namely, a population of polynucleotides that are replicated from one or more starting sequences. Amplifying may refer to a variety of amplification reactions, including but not limited to polymerase chain reaction (PCR), linear polymerase reactions, nucleic acid sequence-based amplification, rolling circle amplification and like reactions. Typically, amplification primers are used for amplification, the result of the amplification reaction being an amplicon.

“Sequencing primers” generally refer to single stranded nucleotide sequences which can prime the synthesis of DNA and are used to sequence DNA. An amplification primer may also be used as a sequencing primer. A sequencing primer can be used as an amplification primer. A sequencing primer hybridizes to the DNA, i.e. base pairs are formed. Nucleotides that can form base pairs, that are complementary to one another, are e.g., cytosine and guanine, thymine and adenine, adenine and uracil, guanine and uracil. The complementarity between the amplification primer and the existing DNA strand does not have to be 100%, i.e., not all bases of a primer need to base pair with the existing DNA strand. The sequence of the existing DNA strand, e.g., sample DNA or an adapter ligated DNA fragment, to which a sequencing primer (partially) hybridizes is often referred to as sequencing primer binding site (SEQ). From the 3′-end of a sequencing primer hybridized with the existing DNA strand, nucleotides are incorporated using the existing strand as a template (template directed DNA synthesis). The incorporation of a particular nucleotide (A, T, C, or G) can be detected during the synthesis, e.g. in pyrosequencing or when fluorescently labelled nucleotides are used. Alternatively, a chain termination method can be used, e.g., Sanger sequencing or Dye termination sequencing. In any case, these and other methods may be contemplated, as long as the order of the nucleotides of a DNA template may be determined by synthesizing DNA with a sequencing primer and detecting incorporated nucleotides and/or synthesized fragments.

An “adapter,” as referred to herein, generally refers to a short double-stranded DNA molecule with a limited number of base pairs, e.g. about 10 to about 100 base pairs in length, which can be designed such that they can be ligated to the ends of DNA fragments or amplicons. Adapters are generally composed of two synthetic oligonucleotides which have nucleotide sequences which are at least partially complementary to each other. An adapter may have blunt ends, may have staggered ends, or a blunt end and a staggered end. A staggered end is a ‘3′ or ‘5′ overhang. When mixing the two synthetic oligonucleotides in solution under appropriate conditions, they will anneal to each other forming a double-stranded structure. After annealing, one end of the adapter molecule may be designed such that it is compatible with the end of a restriction fragment and can be ligated thereto; the other end of the adapter can be designed so that it cannot be ligated, but this does need not to be the case, for instance when an adapter is to be ligated in between DNA fragments. In certain cases, adapters can be ligated to fragments to provide for a starting point for subsequent manipulation of the adapter-ligated fragment, for instance for amplification or sequencing. In the latter case, so-called sequencing adapters may be ligated to the fragments.

“Sequencing” may generally refer to determining the order of. (A) nucleotides (base sequences) in a nucleic acid sample, e.g., DNA or RNA; or determining the order of (B) amino acids in all or part of a polymer, such as a protein, peptide, or other multimeric molecule. Many techniques are available, such as Sanger sequencing or High Throughput Sequencing technologies (HTS). Sanger sequencing may involve sequencing via detection through (capillary) electrophoresis, in which up to 384 capillaries may be sequence analyzed in one run. High throughput sequencing involves the parallel sequencing of thousands or millions or more sequences at once. HTS can be defined as Next Generation sequencing (NGS), i.e. techniques based on solid phase pyrosequencing or as Next-Next Generation sequencing based on single nucleotide real time sequencing (SMRT). HTS technologies are available such as offered by Roche, lllumina and Applied Biosystems (Life Technologies). Further high throughput sequencing technologies are described by and/or available from Helicos, Pacific Biosciences, Complete Genomics, Ion Torrent Systems, Oxford Nanopore Technologies, Nabsys, ZS Genetics, GnuBio. Each of these sequencing technologies have their own way of preparing samples prior to the actual sequencing step. These steps may be included in the high throughput sequencing method. In certain cases, steps that are particular for the sequencing step may be integrated in the sample preparation protocol prior to the actual sequencing step for reasons of efficiency or economy. For instance, adapters that are ligated to fragments may contain sections that can be used in subsequent sequencing steps (so-called sequencing adapters). Or primers that are used to amplify a subset of fragments prior to sequencing may contain parts within their sequence that introduce sections that can later be used in the sequencing step, for instance by introducing through an amplification step a sequencing adapter or a capturing moiety in an amplicon that can be used in a subsequent sequencing step. Depending also on the sequencing technology used, amplification steps may be omitted.

As used herein, the abbreviations for the natural 1-enantiomeric amino acids are conventional and can be as follows: alanine (A, Ala); arginine (R, Arg); asparagine (N, Asn); aspartic acid (D, Asp); cysteine (C, Cys); glutamic acid (E, Glu); glutamine (Q, Gin); glycine (G, Gly); histidine (H, His); isoleucine (I, He); leucine (L, Leu); lysine (K, Lys); methionine (M, Met); phenylalanine (F, Phe); proline (P, Pro); serine (S, Ser); threonine (T, Thr); tryptophan (W, Trp); tyrosine (Y, Tyr); valine (V, Val). Unless otherwise specified, X can indicate any amino acid. In some aspects, X can be asparagine (N), glutamine (Q), histidine (H), lysine (K), or arginine (R). References to these amino acids are also in the form of “[amino acid][residues/residues]” (e.g., lysine residue, lysine residues, leucine residue, leucine residues, etc.).

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below.

### High Throughput Single-Molecule Protein Sequencing

The present disclosure offers a novel approach to sequence proteins and peptides at the single molecule level in a high-throughput fashion. Systems and methods of the present disclosure involve a unique Barcode Transfer Reagent (BTR) that conjugates to peptides, comprises barcode information, and removes the terminal amino acids. The ability to sequence peptides with single molecule sensitivity is expected to provide breakthroughs in proteomic research as well as the study and treatment of diseases. Numerous approaches have been proposed for single molecule peptide sequencing, but these methods suffer from inefficient access of reagents to target peptides and the use of low through-put readout methods. In contrast, by providing an approach to barcode individual amino acids in peptides for removal and ex-situ analysis via methods such as DNA sequencing, the methods and systems of the present disclosure overcome these challenges.

In an aspect, provided herein is a method for processing a peptide or protein, comprising providing the peptide or protein coupled to a barcode, wherein the peptide or protein comprises a terminal amino acid; contacting the terminal amino acid or derivative thereof with a barcode transfer reagent (BTR) to generate a barcoded amino acid complex (BTR-AC), and cleaving the BTR-AC from the peptide or protein. In some embodiments, the BTR-AC comprises barcode information. In some embodiments, the method further comprises transferring or copying the barcode information from the BTR to the barcode of the peptide or protein. In some embodiments, the method comprises transferring or copying the barcode of the peptide or protein to the BTR. In some embodiments, the method further comprises repeating one or more operations at least once to generate a plurality of BTR-ACs. In some embodiments, the method further comprises contacting the BTR-ACs or the plurality of BTR-ACs with a binding agent. The binding agent may be specific or partially specific to the terminal amino acid, the BTR-AC, or a portion of the BTR-AC. In some embodiments, the method further comprises sorting the BTR-AC or plurality of BTR-ACs into groups. In some embodiments, the BTR-ACs are sorted by the terminal amino acid or derivative thereof comprised within the BTR-AC. In some embodiments, the method further comprises copying or transferring a binding agent barcode to the BTR-AC or derivative thereof. In some embodiments, the method further comprises reading out barcode information (e.g., via DNA sequencing) from the BTR-ACs or derivatives thereof and identifying the terminal amino acid or terminal amino acids, thereby sequencing the peptide.

Peptides: The peptide or protein may be derived from a sample, such as a biological sample, As described elsewhere herein, the biological sample may comprise a cell, tissue, cell suspension, culture of cells, a bodily fluid, or an environmental sample. In some examples, the tissue sample may comprise a biopsy. Examples of bodily fluids include blood, serum, plasma, urine, saliva, stool, lavage, cerebrospinal fluid. Examples of environmental samples may include sewage samples.

The peptide or protein may be processed to generate a barcoded peptide or protein (e.g., a peptide or protein coupled to a barcode). Example methods and processing operations are described elsewhere herein and include extraction of the peptide or protein from a sample, de-aggregation of proteins from the sample, isolation of cells or proteins, enrichment, fragmentation, and barcoding of the peptide or protein.

Barcodes: The peptide or protein and the BTR may comprise or be coupled (e.g., covalently or non-covalently) to a barcode. As described elsewhere herein, a barcode may comprise any useful molecule, such as a protein or peptide, nucleic acid molecule, lipid, detectable tag (e.g., fluorophore, mass tag, heavy metal tag, radioisotope, chromogenic enzyme), chemical moiety, or other label. In some embodiments, the proteins in a cell can be tagged with a barcode that has a tag that enables visualization (e.g., fluorescent proteins, dyes). Use of optically detectable tags may allow for tracking and detection of movement of the tagged protein using microscopy (e.g., light microscopy, super-resolution microscopy, fluorescent microscopy). The peptide and the BTR may comprise the same type of barcode or different types of molecules. For example, the peptide may comprise a peptide barcode, while the BTR comprises a nucleic acid barcode molecule. In some instances, the peptide and the BTR comprise the same type of barcode molecules that are distinct from one another, e.g., two nucleic acid barcode molecules with different sequences. In such examples, the peptide or BTR barcodes may comprise additional useful sequences, such as UMIs, primer sequences, cleavage sequences or sites, or encoded information, such as the cycle or round (iteration) number for which the barcode is provided, proximity to a location, structure, interactions, molecular type or characteristics, or a combination thereof, as described elsewhere herein. In some examples, the BTR comprises a nucleic acid barcode molecule comprising a primer sequence and cycle or round information. In some examples, the peptide barcode comprises a primer sequence and a peptide-identifying barcode sequence. In some examples, the primer sequence of the BTR is complementary to the primer sequence of the peptide barcode. In other examples, the BTR comprises a nucleic acid barcode molecule comprising a primer sequence, and the peptide barcode comprises a primer sequence that is complementary to that of the BTR. In some examples, a temporal barcode comprising cycle or round information may be provided that can couple to the BTR, the peptide barcode, or both.

The barcode molecules described herein may comprise multiplexed information. For example, the nucleic acid barcode molecule conjugated to the peptide or as part of the BTR may comprise sequences that encode cycle or other temporal information or spatial information. In one such example, an array of peptides may be provided on a substrate. The array may comprise a plurality of individually addressable units, in which each (or a subset of) individually addressable units of the array comprises a peptide to be analyzed. The peptides, BTRs, or binding agents may comprise spatial information (e.g., spatial barcode sequences) which uniquely identify the individually addressable units and thus the location of the array. The BTRs may additionally comprise temporal information, e.g., a cycle barcode that indicates the round or iteration in which the BTR is provided. Subsequent sequencing of the barcode molecules may be used to reveal the spatial information (e.g., the originating location in the array of a peptide or amino acid). In some instances, the barcode molecules comprise a unique molecular identifier (UMI), which may be used to determine the quantity of a given BTR or amino acid of a given peptide, substrate, array, or sample.

Barcode Transfer Reagents: The BTR may couple or bind to the terminal amino acid of the peptide or protein to generate the BTR-AC. The coupling of the BTR to the terminal amino acid may be covalent or noncovalent. In an example, a BTR may comprise a chemical moiety that is able to react to a terminal amino acid of the peptide and optionally, cleave the amino acid from a peptide. For example, the chemical moiety may be a thiocyanate conjugate, e.g., an isothiocyanate (ITC) such as phenyl isothiocyanate (PITC), 3-pyridyl isothiocyanate (PYITC), 2-piperidinoethyl isothiocyanate (PEITC), 3-(4-morpholino) propyl isothiocyanate (MPITC), 3-(diethylamino)propyl isothiocyanate (DEPTIC) or naphthylisothiocyanate (NITC), ammonium thiocyanate, potassium thiocyanate, trimethylsilyl isothiocyanate (TMS-ITC), phenyl phosphoroisothiocyanatidate, acetyl isothiocyanate (AITC), or an aldehyde group, e.g., ortho-phthalaldehyde (OPA), 2,3-naphthalenedicarboxyaldehyde (NDA), 2-pyridinecarboxyaldehyde, dinitrofluorobenzene (DNFB), dansyl chloride, or other moiety which can react with an N-terminal amino acid (NTAA).

The chemical moiety of the BTR may be an amino acid-reactive moiety. The amino acid-reactive moiety of the BTR may be any useful moiety that enables the reactive moiety to conjugate to and optionally cleave an amino acid. In some examples, the first reactive moiety can react with a terminal amino acid (e.g., NTAA or CTAA). In such examples, the first reactive moiety may comprise any primary amine or carboxylic group reactive group, including but not limited to isocyanates, acyl azides, NHS esters, sulfonyl chlorides, aldehydes, glyoxals, epoxides, oxiranes, carbonates, aryl halides, imidoesters, carbodiimides, anhydrides, phenyl esters, isothiocyanates (e.g., phenyl isothiocyanate, sodium isothiocyanate, ammonium isothiocyanates (e.g., tetrabutylammonium isothiocyanate, tetrabutylammonium isothiocyanate), diphenylphosphoryl isothiocyanate), acetyl chloride, cyanogen bromide, carboxypeptidases, azide, alkyne, DBCO, maleimide, succinimide, thiol-thiol disulfide bonds, tetrazine, TCO, vinyl, methylcyclopropene, acryloyl, allyl, among others. Additional examples of amino acid reactive groups are provided in U.S. Pat. Pub. No. 2020/0217853, which is incorporated by reference herein in its entirety.

The chemical moiety of the BTR may be or comprise a thiol, amine, or click chemistry moiety, which can allow for coupling or conjugation to peptides that are functionalized, e.g., with a thiol group to allow for disulfide bonding between the BTR and peptide, through amide coupling, or through complementary click chemistry reactions.

The BTR may additionally comprise a barcode molecule. As described herein, the barcode molecule may comprise any useful type of molecule, e.g., nucleic acid molecule, lipid, carbohydrate, peptide, polymer, or detectable tag (e.g., fluorophore, mass tag, hapten). In some instances, the barcode molecule comprises a nucleic acid molecule. The nucleic acid molecule, as described elsewhere herein, may comprise RNA, DNA, modified nucleotides, or a combination thereof. The nucleic acid molecule may comprise encoded barcode information, such as a cycle or round number according to the order or round a BTR is provided. For instance, a first BTR comprising cycle information (e.g., Cycle 1) may be provided to react with a N-terminal amino acid (NTAA). Subsequently, a second BTR comprising cycle information (e.g., Cycle 2) may be provided to react with the n-1 NTAA. Accordingly, each iteration or cycle may be tracked using the barcode information and may be used to determine the order (or sequence) in which a particular amino acid occurs in the peptide. Alternatively, or in addition to, a separate temporal barcode (e.g., nucleic acid barcode molecule encoding cycle or round information) may be provided that can couple to the BTR, the peptide barcode, or both.

The barcode molecule, e.g., nucleic acid barcode molecule, of the BTR may additionally comprise a primer sequence. The primer sequence of the BTR may be configured to couple to a primer sequence of the peptide barcode. The coupling may occur via ligation (e.g., via sticky-end or blunt-end ligation), hybridization (e.g., complementary sequences on the BTR primer sequence and the peptide barcode primer sequence) and optionally extended, e.g., using a polymerizing enzyme (e.g., polymerase). The coupling of the BTR primer sequence to the peptide barcode primer sequence can allow for copying or transfer of information of one barcode sequence to another, e.g., from the BTR to the peptide barcode, or from the peptide barcode to the BTR.

In some instances, the barcode molecule of the BTR may be attached to a BTR precursor to generate the BTR comprising the barcode molecule. For example, the BTR precursor may comprise an additional chemical moiety or reactive group that is capable of coupling, either directly or indirectly, to the barcode molecule. In an example of direct coupling, the barcode molecule may comprise a click chemistry moiety (e.g., alkyne, such as DBCO), and the additional chemical moiety of the BTR precursor may comprise an additional click chemistry moiety (e.g., azide) that can react with the click chemistry moiety of the barcode molecule. Alternatively, the BTR precursor may be coupled indirectly to the barcode molecule, e.g., via noncovalent interaction (e.g., avidin or streptavidin with biotin interaction) or via an intermediate linking molecule.

When applicable, the click chemistry moieties of the BTR, barcode molecule, or intermediate linking molecule may comprise any suitable bioorthogonal moieties, as described elsewhere herein, e.g., alkenes, alkynes (e.g., cyclooctynes or derivatives thereof, e.g., aza-dimethoxycyclooctyne (DIMAC), symmetrical pyrrolocyclooctyne (SYPCO), pyrrolocyclooctyne (PYRROC), difluorocyclooctyne (DIFO), α,α-bis(trifluoromethyl)pyrrolocyclooctyne (TRIPCO), bicyclo[6.1.0]nonyne (BCN), dibenzocyclooctyne (DIBO), difluorobenzocyclooctyne (DIFBO), dibenzoazacyclo-octyne (DBCO), difluoro-aza-dibenzocyclooctyne (F2-DIBAC), biaryl-azacyclooctynone (BARAC), difluorodimethoxydibenzocyclooctynol (FMDIBO), difluorodimethoxydibenzocyclooctynone (keto-FMDIBO), and 3,3,6,6-tetramethylthiacycloheptyne (TMTH)), azides, epoxides, amines, thiols, nitrones, isonitriles, isocyanides, aziridines, activated esters, and tetrazines, and combinations, variations, or derivatives thereof. The click chemistry moieties may be subjected to conditions sufficient to react a first click chemistry moiety to a second click chemistry moiety, e.g., provision of metal catalysts, appropriate solvents, pH, temperature, ionic concentration, or light/energy for any useful duration of time.

The BTR may comprise any additional useful moiety. For example, the BTR may comprise a releasable or cleavable moiety. Such a releasable or cleavable moiety may comprise, for example, a disulfide bond, which may be releasable by contacting with a reducing agent (e.g., DTT, TCEP). The BTR may additionally or alternatively comprise any number of spacing moieties, e.g., polymers (e.g., PEG, PVA, polyacrylamide), aminohexanoic acid, nucleic acids, alkyl chains, etc. Such spacing moieties may increase the distance between any other moieties of the BTR, e.g., the amino acid-reactive group and the barcode-reactive group.

In some instances, the coupling or reaction of the BTR to an amino acid (e.g., NTAA or CTAA) changes the chemical structure of the amino acid. For example, if using a BTR comprising an isothiocyanate moiety, the amino acid may be derivatized to a thiocarbamyl group (e.g., under mildly alkaline conditions) during or subsequent to contact with the isothiocyanate moiety. One or more further derivatizations may be performed. For instance, the amino acid or amino acid derivative (e.g., thiocarbamyl-derivatized amino acid) may be further derivatized to a thiazolone group (e.g., under acid conditions), a thiohydantoin group, or other chemical moiety. Similarly, a thiazolone group or thiohydantoin group may be further derivatized to a thiocarbamyl group.

The BTR may comprise a nucleic acid barcode molecule, which may comprise any useful functional sequence. Non-limiting examples of functional sequences include primer sites, UMIs, cleavage sites (e.g., restriction sites), abasic sites, transposition sites, nuclease-recognizing sites, sequencing primer sequences, read sequences, spacer sequences, etc.

Substrates: One or more operations described herein may be performed using a substrate. For example, one or more molecules described herein (e.g., barcoded peptide, BTR, or binding agent) may be coupled to a substrate. In some instances, the peptide, the peptide barcode, or both may be provided coupled to one or more substrates. In some instances, the binding agent is coupled to a substrate.

The substrate may be made from any suitable material, e.g., glass, silicon, gel, polymer, etc., as is described elsewhere herein. In some instances, the substrate may be a bead or a gel bead (e.g., polyacrylamide, agarose, or TentaGel® bead). The substrate may be functionalized. One or more molecules, e.g., a peptide, a binding agent, a barcode, may be coupled to the substrate via a covalent or non-covalent interaction. The molecules can be coupled to the substrate using any suitable chemistry, e.g., click chemistry moieties (e.g., alkyne-azide coupling), photoreactive groups (e.g., benzophenone, phenyldiazirine, phenylazide), 1-ethyl-3-(3-dimethylaminopropyl)carbodiimide hydrochloride (EDC) (e.g., to couple amino-oligos or peptides), N-hydroxysulfosuccinimide (NHS), Sulfo-NHS, or NHS-esters (e.g., to couple sulfhydryl oligos), maleimides, thiols, biotin-streptavidin interactions, cystamine, glutaraldehyde, formaldehyde, succinimidyl 4-(N-maleimidomethyl)cy clohexame-1-carboxylate (SMCC), Sulfo-SMCC, 4-(4,6-Dimethoxy-1,3,5-triazin-2-yl)-4-methylmorpholinium chloride (DMTMM), silane (e.g., amino silanes), combinations thereof, etc. In some instances, the substrate may be functionalized to comprise a coupling chemistry to couple the peptide, the BTR, or both. In one non-limiting example, a substrate (e.g., bead or surface) may comprise an alkyne such as dibenzocyclooctyne (DBCO, e.g., DBCO-alcohol, DBCO-Boc, DBCO-NHS, DBCO-silane), which may be configured to react to an amine, a carboyxl or carbonyl, a sulfhydryl, etc. A DBCO-functionalized substrate may conjugate to a barcode molecule, e.g., an azide-functionalized barcode molecule, which may then subsequently be coupled to a peptide to generate a barcoded peptide. In other examples, linkers such as bifunctional linkers may be used to attach a molecule to a substrate; such bifunctional linkers may comprise the same reactive moiety on both ends or a different moiety at each end (e.g., heterobifunctional linker).

Transfer: In some instances, the barcode information from the BTR is transferred to the barcode of the peptide or protein, or the barcode of the peptide or protein is transferred to the BTR. Transfer of information may occur by coupling of the BTR barcode information to the peptide barcode. For example, the BTR may comprise a nucleic acid barcode molecule comprising a primer sequence and, in some embodiments, encoded information, e.g., cycle or round information. Similarly, the peptide barcode may comprise a nucleic acid barcode molecule that identifies the peptide or the sample, partition, or cell from where the peptide originated. The peptide barcode may comprise an additional primer sequence. The primer sequence of the BTR may be complementary and hybridize to the additional primer sequence of the peptide barcode. Information from the BTR (e.g., cycle number) may be transferred to the peptide barcode, e.g., by performing an extension reaction (e.g., using a polymerase). Alternatively, information from the peptide barcode may be transferred to the BTR. Alternatively, or in addition to, the BTR nucleic acid molecule may be coupled to the peptide barcode via a splint or bridge oligo, ligation (e.g., blunt-end ligation, ligation of hybridized products), or both. In some instances, a temporal barcode (e.g., comprising round or cycle information) may be provided as a separate molecule which can couple, e.g., via hybridization, ligation, or via a splint molecule, to the BTR, the peptide barcode, or both. In some instances, the information transfer between the BTR and the peptide barcode may occur via nucleic acid recombination (e.g., using a recombinase, Cas9, or other endonuclease). In some instances, the information transfer may occur by toehold-mediated strand displacement and optional ligation.

Subsequent to transfer or coupling, the resultant nucleic acid molecule may be subjected to amplification. Amplification may be performed using any useful technique, such as polymerase chain reaction (PCR), linear polymerase reactions, nucleic acid sequence-based amplification, rolling circle amplification, loop-mediated isothermal amplification, helicase-dependent amplification, multiple displacement amplification, strand invasion based amplification, strand displacement amplification, recombinase polymerase amplification, nicking enzyme amplification reaction, nucleic acid sequence-based amplification, gp32-based amplification, and similar reactions. An amplification reaction may generate an amplicon. An amplification reaction may be performed isothermally or may require temperature changes.

Cleaving: The cleaving of the terminal amino acid or BTR-AC from the peptide may be achieved using any suitable mechanism, such as via application of a stimulus. The stimulus can be, for example, a chemical stimulus, a biological stimulus, a thermal stimulus (e.g., application of heat), a photo-stimulus, a physical or mechanical stimulus, or other type of stimulus or a combination of stimuli. In some instances, the stimulus may be a chemical stimulus, e.g., a change in pH (e.g., acidic or basic cleavage), addition of a lytic agent, initiating agent, radical-generating agent, reducing agent, etc. In some instances, the stimulus may be a biological stimulus, e.g., enzyme (e.g., Edmanase, protease, endonuclease) that can cleave or catalyze cleavage of the terminal amino acid or BTR-AC from the peptide.

In some examples, the BTR-AC comprises an amino acid reactive group (e.g., PITC) and cleavage of the BTR-AC from the peptide is achieved using a stimulus (e.g., change in pH, temperature). In one such example, the BTR comprises an isothiocyanate moiety (e.g., PITC moiety) that can couple to an N-terminal amino acid (NTAA) under mildly alkaline conditions to generate a phenylthiocarbamoyl (PTC) derivative of the NTAA, and cleavage of the NTAA from the peptide may be achieved using an Edman degradation reaction (e.g., application of an acid such as trifluoroacetic acid with heat), to generate a thiazolinone (ATZ) derivative or a phenylthiohydantoin (PTH) derivative.

In some instances, more than one terminal amino acid may be cleaved from the peptide per cleavage event. The cleaving may comprise cleaving 2 terminal amino acids, 3 terminal amino acids, 4 terminal amino acids, 5 terminal amino acids, 6 terminal amino acids, 7 terminal amino acids, 8 terminal amino acids, 9 terminal amino acids, 10 terminal amino acids, or more. For example, the peptide may comprise a peptide comprising a plurality of amino acid terminal amino acids, and single amino acids, di-peptides, tri-peptides, quadri-peptides, or larger may be cleaved in the methods described herein. In some instances, at most about 10 terminal amino acids, at most about 9 terminal amino acids, at most about 8 terminal amino acids, at most about 7 terminal amino acids, at most about 6 terminal amino acids, at most about 5 terminal amino acids, at most about 4 terminal amino acids, at most about 3 terminal amino acids, or fewer terminal amino acids may be cleaved in a given cleavage event. In some instances, cleavage of greater than one terminal amino acid (e.g., amino acid) may be mediated using an enzyme (e.g., Edmanase, protease) that is capable of recognizing or cleaving more than a single amino acid.

Cleavage of the terminal amino acid (or plurality of terminal amino acids) may be conducted using a biological stimulus, such as an enzyme. The enzyme can be any useful cleaving enzyme, e.g., a protease, such as an Edmanase, cruzain, a cleaving protein (e.g., ClpS, ClpX), Proteinase K, exopeptidase, aminopeptidase, diaminopeptidase, serine protease, cysteine protease, threonine protease, aspartic protease, aspartic protease, glutamic protease, metalloprotease, asparagine peptide lyase, pepsin, trypsin, pancreatin, Lys-C, Glu-C, Asp-N, chymotrypsin, carboxypeptidase (e.g., carboxypeptidase A, carboxypeptidase B, carboxypeptidase Y), SUMO protease, elastase, papain, endoproteinase, proteinase, TrypZean®, bromelain, collagenase, hyaluronase, thermolysin, ficin, keratinase, tryptase, fibroblast activation, enterokinase, chymotrypsinogen, chymase, clostripain, calpain, alpha-lytic protease, proline specific endopeptidase, furin, thrombin, subtilisin, genenase, PCSK9, cathepsin, prolidase, methionine aminopeptidase, cathepsin C, 1-cyclohexen-1-yl-boronic acid pinacol ester, pyroglutamate aminopeptidase, renin, kininogen, kallikrein, DPPIV/CD26, thimet oligopeptidase, prolyl oligopeptidase, leucine aminopeptidase, dipeptidylpeptidase, or other enzyme or protease, or a combination or variation (e.g., engineered mutant or variant) thereof.

In the instances of enzymatic cleavage, additional reagents may be provided to catalyze or induce the cleavage. For instance, metalloproteases, aminopeptidases, or exopeptidases may facilitate cleavage of an amino acid or plurality of amino acids in the presence of a catalyst, e.g., metal or metal ion (e.g., cobalt). Accordingly, a catalyst may be provided in order to facilitate the binding of the enzyme to an amino acid or the subsequent cleavage of the amino acid from the peptide. In some examples, cleavage may be mediated by an apo-enzyme, which is inactive in the absence of a metal catalyst of cofactor, and cleavage may be controlled by addition of metal or metal ions.

Other examples of cleaving stimuli include: a photo stimulus (e.g., application of UV, X-rays, gamma rays, or other wavelength of light), mechanical stimulus (e.g., sonication, high pressure, electromagnetic energy), thermal stimulus (e.g., application of heat), or chemical stimulus. In some instances, the peptide may comprise or be altered to comprise a cleavable or labile bond that can be cleaved upon application of the appropriate stimulus, e.g., disulfide bonds (e.g., cleavable upon application of a chemical stimulus such as a reducing agent), ester linkages (e.g., cleavable with a change of pH), a vicinal-diol linkage (e.g., cleavable with sodium periodate), a Diels-Alder linkage (e.g., cleavable upon application of heat), a sulfone linkage (e.g., cleavable via a base), a silyl ether linkage (e.g., cleavable via an acid), a glycosidic linkage (e.g., cleavable via an amylase), a peptide linkage (e.g., cleavable via a protease), or a phosphodiester linkage (e.g., cleavable via a nuclease (e.g., DNase)).

Binding agents: The binding agent may be contacted with the BTR-AC prior to, during, or subsequent to cleavage of the BTR-AC from the peptide. The binding agent may be any useful molecule that can couple to the amino acid or BTR-AC. For example, a binding agent may be or comprise a protein or peptide (e.g., an antibody, antibody fragment, single chain variant fragment (scFv), nanobody, anticalin, tRNA synthetase or tRNA-acyl transferase, a fibronectin domain), a peptide mimetic, a peptidomimetic (e.g., a peptoid, a beta-peptide, a D-peptide peptidomimetic), an artificial protein, artificial peptide, or artificial motif, a polysaccharide, a nucleic acid molecule (e.g., aptamer), a somamer, a polymer, an inorganic compound, an organic compound, a small molecule, or derivatives (e.g., engineered variants) or combinations thereof. The binding agent may comprise one or more components or separate binding agents that are linked or fused together to generate a multimeric binding agent. The multimeric unit may recognize a single binding partner or the sum of binding partners of the individual components. The binding agent may be able to bind to a modified amino acid (e.g., an amino acid coupled to a linker or a BTR, a post-translationally modified amino acid) or portion thereof. The binding agent may comprise a recognition site that specifically recognizes an amino acid, BTR-AC, or a derivatized, and optionally modified, amino acid or BTR-AC. For example, the binding agent may be configured to recognize or have binding specificity to a moiety of a modified amino acid, such as a specific amino acid residue, the BTR-AC, or derivatized amino acid or BTR-AC (e.g., a thiocarbamoyl-derivatized residue, a thiazolone-derivatized residue, a thiohydantoin-derivatized residue, etc.), or a portion thereof. In some instances, the binding agent may be configured to recognize or have binding specificity to a specific post-translational modification. In some instances, the binding agent may be derived or engineered from a naturally-occurring enzyme or protein, e.g., an aminopeptidase, carboxypeptidase, exopeptidase, metalloprotease, antibody, anticalin, N-recognin protein, Clp protease, endoprotease (e.g. trypsin), or tRNA synthetase. In some examples, a binding agent may be a cleaving enzyme (e.g., trypsin, endoprotease) that has been modified to remove the peptidase activity.

The binding agents may be used to capture the BTR-AC or plurality of BTR-ACs, e.g., via pull-down or affinity-based capture. For instance, the binding agents may comprise antibodies that specifically or partially specifically bind to particular amino acid residues or BTR-ACs. In some instances, the binding agents comprise a barcode molecule, e.g., a nucleic acid barcode molecule comprising a barcode sequence. The barcode sequence may encode for the identity of the binding agent or the binding partner. For example, an amino acid or BTR-AC may be contacted with a binding agent (e.g., antibody, antibody fragment, nanobody) that specifically recognizes the amino acid residue, BTR-AC, or derivative thereof (e.g., a PTH, PTC, ATZ derivatized form) over other amino acid residues or derivatives thereof. The nucleic acid barcode molecule may comprise information that identifies the binding agent (e.g., anti-alanine, anti-leucine, anti-glycine, etc.), which, due to the specificity of the binding agent to its target, may also identify the particular amino acid residue (or derivative). The nucleic acid barcode molecule may be directly coupled to the binding agent (e.g., an oligo-conjugated binding agent), or the barcode and the binding agent may be indirectly coupled, e.g., both provided on a substrate (e.g., bead or particle), such that the barcode may be associated with the binding agent.

In some instances, the barcode information of the BTR may be copied or transferred to the nucleic acid barcode molecule of the binding agent, or, alternatively, the nucleic acid barcode molecule of the binding agent may be transferred or copied to the BTR. For example, the binding agent may be coupled to the binding agent nucleic acid barcode molecule (e.g., directly or indirectly, such as via a substrate). The binding agent may recognize and bind the cleaved BTR-AC, and the nucleic acid barcode molecule of the binding agent may couple to the barcode of the BTR-AC. Coupling of the nucleic acid barcode molecule of the binding agent to the barcode information of the BTR-AC may occur, in some examples, through hybridization to one another or to a splint molecule, with optional ligation). A polymerase extension reaction may be performed to transfer information from one barcode to the other (see, e.g., FIGS. 2, and 21-23). Optional amplification operations (e.g., PCR, isothermal amplification) may be performed.

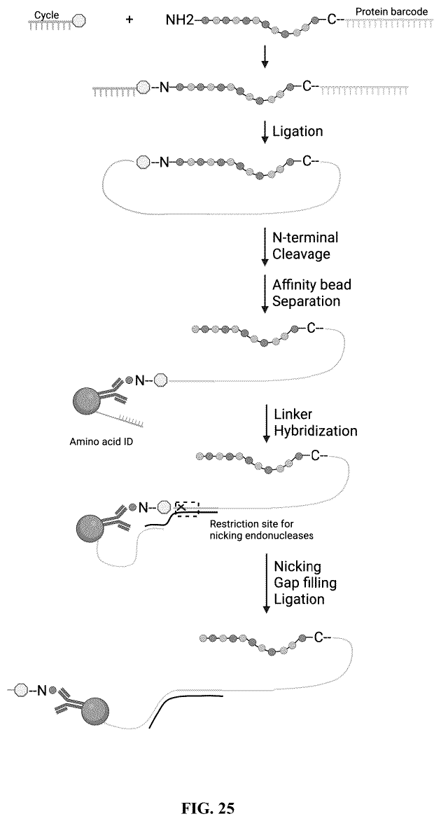

In some instances, the peptide barcode may be transferred to the nucleic acid barcode molecule of the binding agent, or alternatively, the nucleic acid barcode of the binding agent may be transferred to the peptide barcode. In one such example, the BTR-AC may comprise a BTR nucleic acid molecule that is capable of coupling to the protein barcode. Subsequent to cleavage of the BTR-AC, the cleaved BTR-AC may remain coupled to the peptide barcode. The binding agent may be coupled to a binding agent nucleic acid barcode molecule (e.g., directly or indirectly via a substrate). The binding agent may recognize and bind the cleaved BTR-AC, and the nucleic acid barcode molecule of the binding agent may couple to the peptide barcode that is coupled to the BTR-AC. Coupling of the nucleic acid barcode molecule of the binding agent to the peptide barcode or barcode information of the BTR-AC may occur, in some examples, through hybridization to one another or to a splint molecule, with optional ligation). A polymerase extension reaction may be performed to transfer information from one barcode to the other (see, e.g., FIG. 25). Optional amplification operations (e.g., PCR, isothermal amplification) may be performed. In some instances, one of the barcodes (e.g., the peptide barcode, the binding agent barcode, the BTR barcode) comprises a restriction site, which may allow for cleavage of the barcode at a designated region.

A peptide or plurality of peptides may be contacted with a library of binding agents. The library of binding agents may comprise a plurality of binding agents that have specificity to different analytes. For example, the library of binding agents may comprise a plurality of binding agents that recognize different amino acids, BTR-ACs, or derivatives thereof (e.g., derivatized amino acids such as the PTH, PTC, or ATZ forms), clusters of amino acids (e.g., dipeptides, tripeptides, etc.), or combinations of amino acids (e.g., amino acids with similar side chain groups). In one such example, a given binding agent may recognize and bind to more than one amino acid, optionally with different affinities or binding kinetics. The given binding agent may recognize and bind to a single amino acid, two different amino acids, three different amino acids, four different amino acids, etc. For instance, a given binding agent may bind to amino acids with similar residues, e.g., amino acids with positively-charged side chains (e.g., arginine, histidine, lysine), negatively-charged side chains (aspartic acid, glutamic acid), amino acids with polar uncharged side chains (e.g., serine, threonine, asparagine, glutamine), amino acids with hydrophobic side chains (e.g., alanine, valine, isoleucine, leucine, methionine, phenylalanine, tyrosine, trytophan), aliphatic side chains (e.g., glycine, alanine, valine, leucine, isoleucine), hydroxyl or sulfur or selenium-containing side chains (e.g., serine, cysteine, selenocysteine, threonine, methionine), aromatic side chains (e.g., phenylalanine, tyrosine, tryptophan), basic side chains (e.g., histidine, lysine, arginine), acidic side chains (e.g., aspartate, glutamate, asparagine, glutamine), or a combination thereof. Altogether, the library of binding agents may specifically recognize or bind to any number of different amino acids; for example, the library of binding agents may be configured to specifically bind to at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 different proteinogenic amino acids or derivatives thereof.

The library of binding agents may comprise any useful number of binding agents, each of which can have different binding specificities. For example, a first binding agent may recognize and one amino acid, and a second binding agent may recognize two amino acids, and a third binding agent may recognize three amino acids. In another example, a first binding agent may recognize one amino acid, a second binding agent may recognize a different amino acid, and a third binding agent may recognize a plurality of amino acids. It will be appreciated that any number of binding agents may be provided, and that each binding agent may have specificity to one or more amino acids. Altogether, the library of binding agents may bind to all 20 proteinogenic amino acids or derivatives thereof, or a subset (e.g., 10 or more, 15 or more) of the amino acids.

In some instances, passivation of a binding agent or of a substrate may be performed prior to or during contact with the cleaved BTR-AC. Passivation may be achieved using a blocking agent or solution, such as milk proteins (e.g., lactoglobulin, lactalbumin, lactoferrin, casein, whey, immunoglobulin, insulin, growth factors, osteopontin), albumin (e.g., bovine serum albumin), Tween 20, commercially available blocking solutions, or a combination thereof. Alternatively, or in addition to, passivation may be performed using a polymer (e.g., polyethylene glycol), organic compound (e.g., oil, lipids), sugar, nanoparticle, inorganic compound, ion, etc.

Sorting: In some instances, sorting of the BTR-ACs may be performed. The BTR-ACs may be sorted by any useful property, e.g., the identity of the terminal amino acid or the side chain of the terminal amino acid (e.g., alanine, leucine, tryptophan, etc.), chemical or physicochemical properties, e.g., charge, size, polarity, side chain types, e.g., hydrophobic side chains, aliphatic side chains, charged side chains, polar side chains, positively or negatively charged side chains, etc. The BTR-ACs may be sorted based on the affinity of the binding agent. For example, binding agents that are specific to a single or subset of amino acids may be bound to their respective BTR-ACs and then sorted into a compartment using affinity-based approaches (e.g., pulldown assays), based on the amino acid identity of the BTR-AC (e.g., alanine, leucine, tryptophan, etc.). Accordingly, each compartment may comprise BTR-ACs having a single amino acid type (or multiple types if the binding agents are specific to more than one amino acid type). Sorting may be performed using any useful approach, e.g., pulldown assays, sorting via magnetism or fluorescence (e.g., MACS or FACS), electrophoresis, chromatography, etc. In some instances, the binding agents are coupled to substrates, such that a single substrate has one or more binding agents that bind to the same target. Sorting of the BTR-ACs may thus be performed by sorting the individual substrates.

Nucleic Acid Sequencing: The nucleic acid molecules (e.g., peptide barcode, BTR barcode, or binding agent barcode) may be subjected to sequencing to determine the identity of the amino acids. For example, following cleavage of the BTR-ACs and contacting the BTR-ACs with binding agents, the nucleic acid molecules (e.g., the BTR barcode, the peptide barcodes) may be subjected to sequencing. In some instances, sequencing can be performed on the peptide barcode after multiple rounds or cycles of barcode transfer (from the BTRs). In some instances, sequencing can be performed on the BTR-ACs subsequent to transfer of the peptide barcode or portion thereof to the BTR-AC. In some instances, the nucleic acid molecules are amplified (e.g., using nucleic acid amplification approaches such as polymerase chain reaction (PCR), isothermal amplification, ligation-mediated amplification, transcription-based amplification, etc.). Amplification may be performed, for example, using the primer sequences on the BTR or the peptide barcode. Alternatively, or in addition to, an adapter sequence comprising a primer binding site may be added to the nucleic acid molecules. Any number of useful preparation operations may be performed, such as purification or enrichment, cleanup, nucleic acid reactions (e.g., ligation, extension, amplification, tagmentation, restriction enzyme cleavage), fragmenting, barcoding, addition of adapters, enzymatic treatment, etc. Sequencing may be performed using a commercially available nanopore system, e.g., Oxford Nanopore Technologies, Genia Technologies, NobleGen, or Quantum Biosystem, or other sequencing and next generation sequencing systems, e.g., Illumina, BGI, Qiagen, ThermoFisher, PacBio, and Roche, including formats such as parallel bead arrays, sequencing by synthesis, sequencing by ligation (e.g., SOLiD), capillary electrophoresis, electronic microchips, “biochips,” microarrays, parallel microchips, single-molecule arrays, and Sanger sequencing, as is described elsewhere herein.

Sequencing may output the identity of the nucleic acid molecules. For example, subsequent to one or more iterations of contacting the BTR to the terminal amino acid, cleavage of the BTR-AC, and optional transfer of information from the BTR to the peptide barcode, the resultant peptide barcode may comprise stacks of nucleic acid sequences obtained from multiple rounds of binding and transfer of barcode information from the BTR-ACs. Alternatively or in addition to, if the peptide barcode is transferred to the BTR-AC, the resultant BTR-AC may comprise (i) the terminal amino acid, (ii) information on the cycle number and (iii) the peptide barcode (or portion thereof). Accordingly, sequencing of the peptide barcode, BTR-AC, or both may yield sequencing reads that identify the information encoded therein, e.g., the peptide barcode, and the cycle number. In instances where the barcode information of the BTR, the binding agent barcode (if present), or the peptide barcode encodes additional information (e.g., comprises UMIs, spatial information etc.), multiple types of information may be revealed from the nucleic acid sequencing of the peptide barcode.

Sequencing reads may be assembled using a de novo approach to identify the peptide or protein. For instance, fragmented peptides arising from a common parent protein may be labeled with a common peptide barcode sequence. Putative peptide reads can thus be assembled based on the common barcode sequence, amino acid identity, and if applicable, cycle number. Erroneous reads may be identified through probabilistic modeling of accuracy of reads, resulting in reconstructed, fragmentary, peptide sequences (contigs) with possible gaps for missed or unidentified rounds/amino acid. An alternative option for de novo read reconstruction may employ end-to-end, unsupervised machine learning based reconstruction of peptide reads. This option may employ a Machine Learning Algorithm, such as a deep-learning based model that takes as its input NGS sequencing reads associated with a parent protein/peptide barcode, and outputs the likely reconstruction of peptide reads (contigs). Training of the model can be conducted with protein sequencing runs using known protein/peptide standards. The de novo reconstruction may output reconstructed, fragmentary, peptide sequences (contigs) with a probability assigned to each amino acid as well as the assembled peptide sequence. In some instances, a k-mer or De Brujin approach may be used for peptide sequence reconstruction. For example, reads arising from each nucleic acid molecule may be broken down into shorter k-mer sequences. The k-mer sequences from the pool of reads may be assembled into longer contig sequences. A De Brujin graph may be generated, e.g., to represent splice variants, post-translational modifications, or other proteoforms. The isoforms may be assembled, and the expression level may be determined using a Bayesian approach. The assembled isoforms of proteins may be subjected to evaluation and error correction, e.g., by comparison with standard proteins that are spiked in samples, and assessing for missing segments of sequences, incorrect or redundant assembly, uniform coverage, etc.

Alternatively or in addition to, the binding agent may comprise a detectable label or moiety. For example, the binding agent may comprise a fluorophore, radioisotope, mass tag, chromogenic enzyme (e.g., horse radish peroxidase), etc., which may be detectable using the appropriate imaging technique. Different binding agents (e.g., binding agents that recognize different amino acids, PTMs, or groups of amino acids or PTMs) may be labeled with distinct labels, e.g., different fluorophores, which can be used to identify the presence of a particular amino acid. In some examples, fluorophore-labelled binding agents can be detected using single molecule imaging (e.g., total internal reflection, confocal, wide-field, or super resolution microscopy (e.g., PALM, STORM, STED)).

In some instances, the binding agent, the BTR-AC, or other molecules may be characterized or analyzed using another detection scheme. In some examples, a nanopore may be used to sequence the nucleic acid barcode molecule of the BTR-AC and optionally, the cleaved terminal amino acid of the BTR-AC. The nanopore may be able to distinguish individual amino acids from other amino acids. Similarly, the nanopore may be used to sequence the barcode information of the BTR-AC to obtain information on the cycle number and originating peptide of a given amino acid.

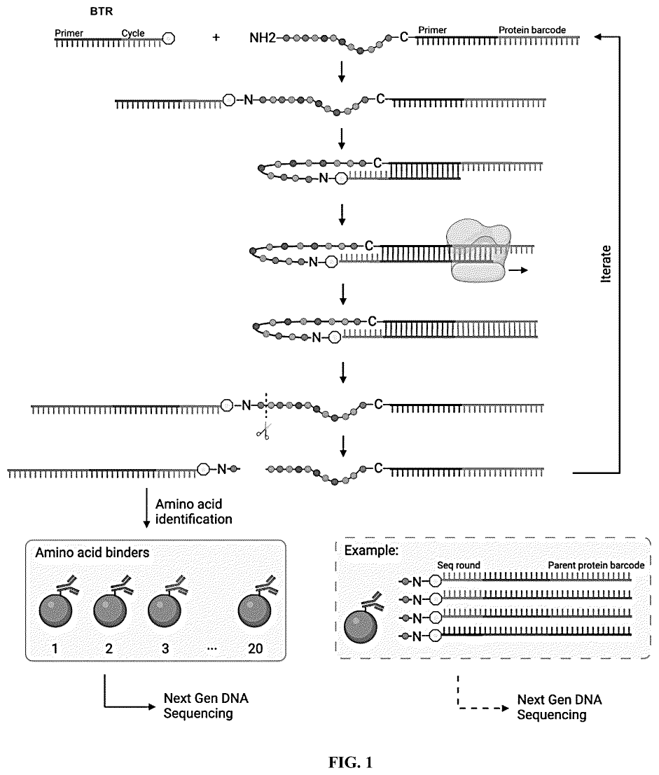

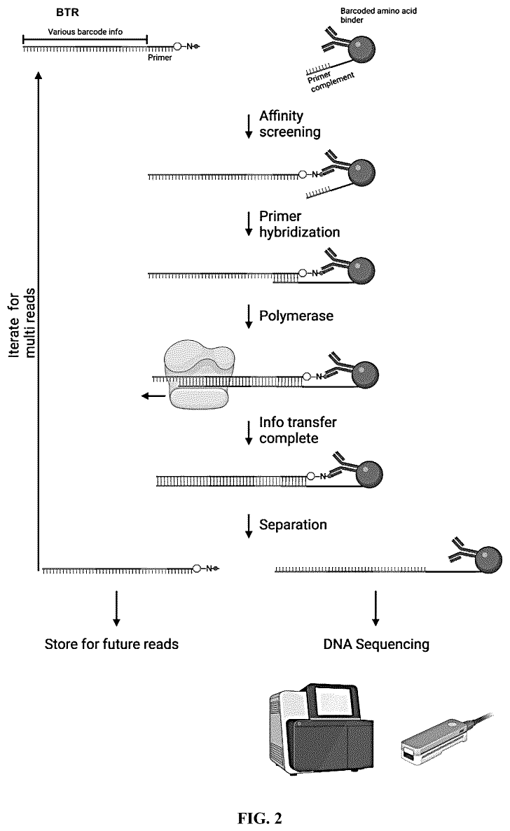

FIG. 1 schematically shows an example of processing and characterizing a peptide. In brief, a peptide is tagged (or provided pre-tagged) with a unique peptide barcode (e.g., a nucleic acid barcode molecule). A BTR comprising barcode information (e.g., a nucleic acid barcode molecule) is provided. The BTR may comprise any useful sequences, such as a primer sequence and in some instances, temporal barcode information, e.g., cycle or round information (e.g., cycle 1, cycle 2, cycle 3, etc.). Alternatively, the temporal barcode information may be provided as a separate molecule (e.g., temporal nucleic acid barcode molecule), which may separately anneal or be ligated to the BTR or the peptide barcode (not shown). The peptide barcode may comprise an additional primer sequence and a barcode that identifies the peptide or the originating parent protein, partition, cell, sample, etc. from which the peptide arises. The BTR conjugates to the terminal end of the protein (the example in FIG. 1 shows sequencing from the N-terminus) to generate a BTR-AC. In some examples, the BTR comprises an amino acid reactive group, e.g., PITC, that can react with the N-terminus of the peptide. Following the conjugation of the BTR to the terminal amino acid of the peptide, the barcode information of the BTR is added (e.g., by copying or transferring) to the peptide barcode or vice versa. Such information transfer may occur via coupling of the nucleic acid barcode molecule of the BTR to the peptide barcode, e.g., via hybridization of complementary primer sequences, hybridization via a splint or bridge molecule (not shown), ligation (not shown), an extension reaction, or a combination thereof. Next, the terminal amino acid is cleaved (e.g., chemically or enzymatically) from the rest of the peptide. In some examples, cleavage may be achieved by using an acid (e.g., trifluoroacetic acid) to cleave the terminal amino acid from the peptide. The liberated BTR-AC can be further processed for downstream analysis.

In some instances, the downstream analysis comprises use of binding agents. The BTR-AC may be contacted with binding agents, such as affinity reagents, that can couple, specifically, partially-specifically, or non-specifically, to the BTR-AC. For example, the binding agents may be specific to one amino acid of the 20 proteinogenic amino acids or to a subset of amino acids of the 20 proteinogenic amino acids. In some instances, the binding agents can bind to particular amino acids or PTMs and can be purified or enriched from a sample using a pull-down assay (e.g., binding agents are attached to magnetic beads that can be pulled down using magnetic force, chromatography, or other separation mechanism, as described elsewhere herein). In some instances, a library of binding agents may be provided and contacted with a plurality of BTR-ACs comprising different barcodes. The library of binding agents may comprise binding agents that are specific to single amino acids or multiple amino acids. Subsequent to binding of the library of binding agents with the BTR-ACs, the binding agents may be sorted into individual populations, e.g., based on the amino acid or sets of amino acids that the binding agent recognizes. Accordingly, each sorted individual population may be assigned and identified as a particular amino acid or set of amino acids, depending on the specificity of the binding agent. In some instances, the binding agents are detected. For example, the binding agents may comprise a detectable label (e.g., fluorophore, radioisotope, mass tag) that can be identified and output the identity of the binding agent and its binding partner (e.g., the terminal amino acid).

In some instances, the information of the BTR-AC can be read out to determine the parent protein and the sequence round or cycle. For example, if the barcode information is DNA based, the peptide barcode may be transferred to the BTR-AC, the BTR-AC may be cleaved from the peptide, and the barcode information of the BTR-ACs can be read out with next generation DNA sequencing, DNA nanopores, or DNA or RNA ligation-based identification (e.g., in situ hybridization, fluorescent probes, microarray analysis, etc.). In some examples, the BTR-ACs comprise a cycle number and the peptide barcode sequence that is transferred or copied from the barcoded peptide; accordingly, sequencing of the BTR-ACs or the nucleic acid molecules of the BTR-ACs can yield information on the originating peptide (from the peptide barcode), the amino acid identity (e.g., as determined from the binding agents), and the cycle number or order in which the amino acid is present in the peptide. Iterative analysis of the individual terminal amino acids can yield full or partial sequence information of the amino acid constituents of the peptide.

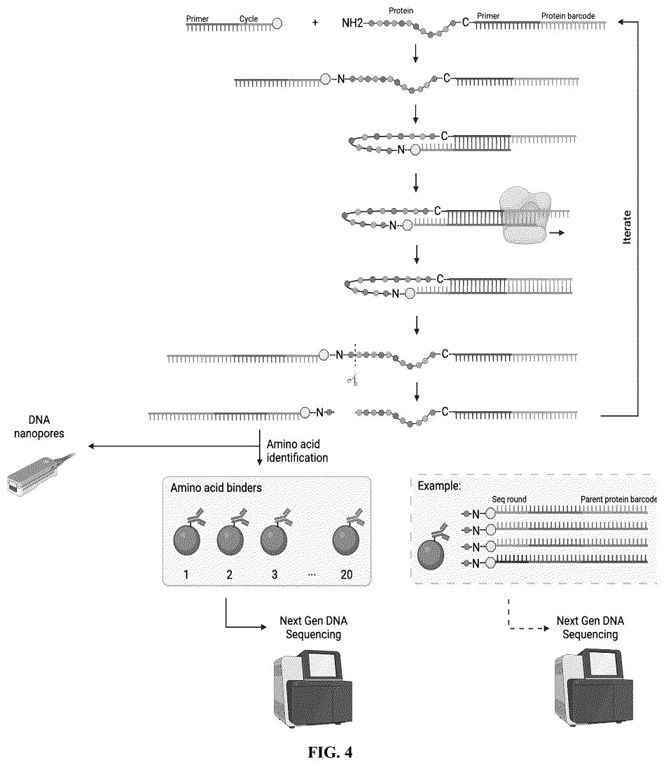

FIG. 4 schematically shows another example of processing and characterizing a peptide. The operations of FIG. 4 may be similar to those described above and presented in FIG. 1. In some instances, following transfer of the peptide barcode to the BTR-AC and cleavage of the BTR-AC from the peptide, the BTR-AC may be further characterized using a nanopore sequencing system. The nanopores may detect and identify the terminal amino acid of the BTR-AC, the nucleic acid barcode molecule of the BTR-AC, or both. Such sequencing can thus identify the identity and cycle number or order in which the detected amino acid is present in a peptide.

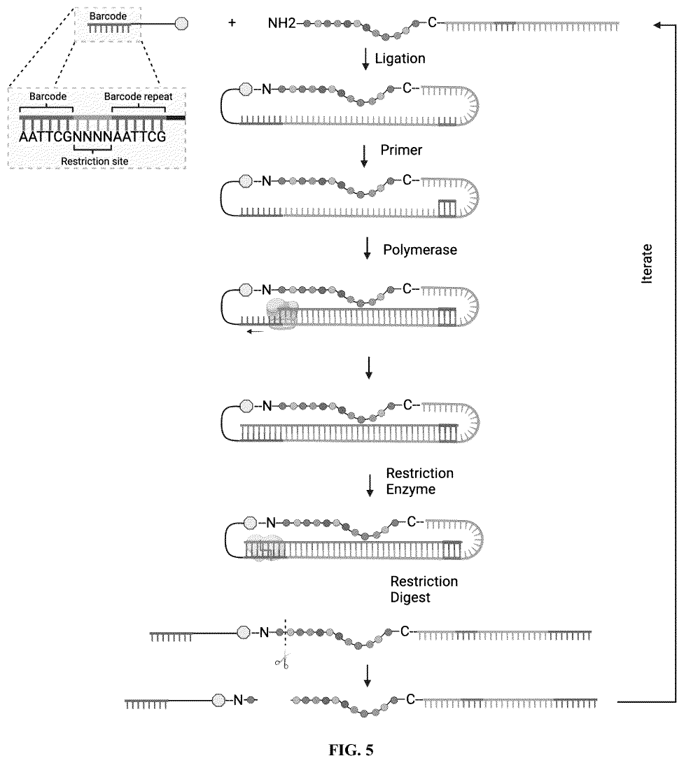

FIG. 5 schematically shows another example of processing and characterizing a peptide. In brief, a peptide is tagged (or provided pre-tagged) with a unique peptide barcode (e.g., a nucleic acid barcode molecule). A BTR comprising barcode information (e.g., a nucleic acid barcode molecule) is provided. The BTR may comprise any useful sequences, such as a barcode, barcode repeat, and a restriction site. The peptide barcode may comprise a primer sequence and a barcode that identifies the peptide or the originating parent protein from which the peptide arises. The BTR conjugates to the terminal end of the protein to generate a BTR-AC. In some examples, the BTR comprises a chemical moiety such as an amino acid reactive group, e.g., PITC, that can react with the N-terminus of the peptide. Following the conjugation of the BTR to the terminal amino acid of the peptide, the barcode information of the BTR is added (e.g., by copying or transferring) to the peptide barcode or vice versa. Such information transfer may occur via coupling of the nucleic acid barcode molecule of the BTR to the peptide barcode, e.g., via ligation, optionally using a splint molecule (not shown). A primer may be provided that can anneal to the primer sequence of the peptide barcode, and an extension reaction may be performed to generate an extended barcoded nucleic acid molecule. Next, the extended barcoded nucleic acid molecule may be cleaved, e.g., using a restriction enzyme. The terminal amino acid is cleaved (e.g., chemically or enzymatically) from the rest of the peptide. The liberated BTR-AC can be further processed for downstream analysis, as described elsewhere herein.

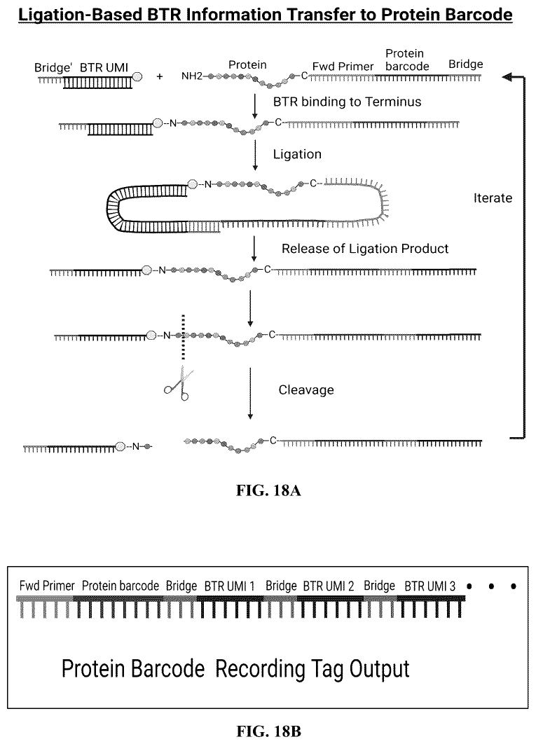

FIG. 18A schematically shows another example of processing and characterizing a peptide. A peptide is tagged (or provided pre-tagged) with a unique peptide barcode (e.g., a nucleic acid barcode molecule). A BTR comprising barcode information (e.g., a nucleic acid barcode molecule) is provided. The BTR may comprise any useful sequences, such as a barcode sequence, a UMI, and a single-stranded overhang or bridge sequence. The peptide barcode may comprise a primer sequence, a barcode that identifies the peptide or the originating parent protein from which the peptide arises, and an additional bridge sequence that may be complementary to the bridge sequence of the BTR. The BTR conjugates to the terminal end of the protein to generate a BTR-AC. In some examples, the BTR comprises an amino acid reactive group, e.g., PITC, that can react with the N-terminus of the peptide. Following the conjugation of the BTR to the terminal amino acid of the peptide, the barcode information of the BTR is added (e.g., by copying or transferring) to the peptide barcode or vice versa. Such information transfer may occur via coupling of the nucleic acid barcode molecule of the BTR to the peptide barcode, e.g., via hybridization of the two complementary bridge sequences of the BTR and peptide barcode. The bridge sequence may also act as a primer sequence to perform an extension reaction. Alternatively, the primer sequence of the peptide barcode may be used to prime the extension reaction. The extended barcoded nucleic acid molecule may be cleaved, e.g., using a restriction enzyme. The terminal amino acid is cleaved (e.g., chemically or enzymatically) from the rest of the peptide. The liberated BTR-AC can be further processed for downstream analysis, as described elsewhere herein. FIG. 18B schematically shows the output of multiple iterations of the workflow outlined in FIG. 18A.

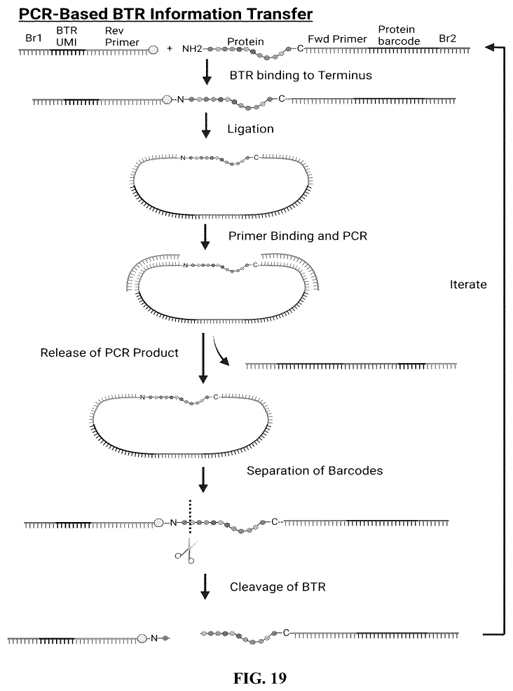

FIG. 19 schematically shows another example of processing and characterizing a peptide. A peptide is tagged (or provided pre-tagged) with a unique peptide barcode (e.g., a nucleic acid barcode molecule). A BTR comprising barcode information (e.g., a nucleic acid barcode molecule) is provided. The BTR may comprise any useful sequences, such as a first barcode sequence, a UMI, and a first primer sequence (e.g., a reverse primer). The peptide barcode may comprise a second primer sequence (e.g., a forward primer), a barcode that identifies the peptide or the originating parent protein from which the peptide arises, and optionally an additional barcode sequence. The BTR conjugates to the terminal end of the protein to generate a BTR-AC. In some examples, the BTR comprises an amino acid reactive group, e.g., PITC, that can react with the N-terminus of the peptide. Following the conjugation of the BTR to the terminal amino acid of the peptide, the barcode information of the BTR is added (e.g., by copying or transferring) to the peptide barcode or vice versa. Such information transfer may occur via coupling of the nucleic acid barcode molecule of the BTR to the peptide barcode, e.g., via ligation of two end sequences of the peptide barcode and the BTR. Primers may be provided that can hybridize to the first primer sequence and the primer sequence, and an extension reaction may be performed. The extended barcoded nucleic acid molecule may be cleaved, e.g., using a restriction enzyme. The terminal amino acid is cleaved (e.g., chemically or enzymatically) from the rest of the peptide. The liberated BTR-AC can be further processed for downstream analysis, as described elsewhere herein.

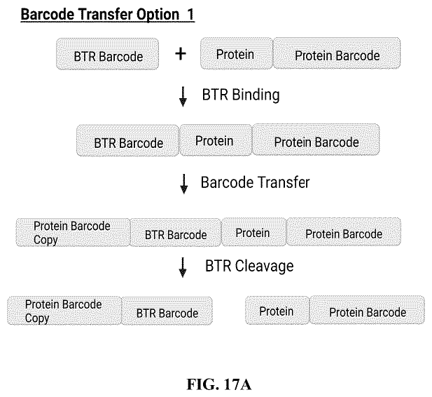

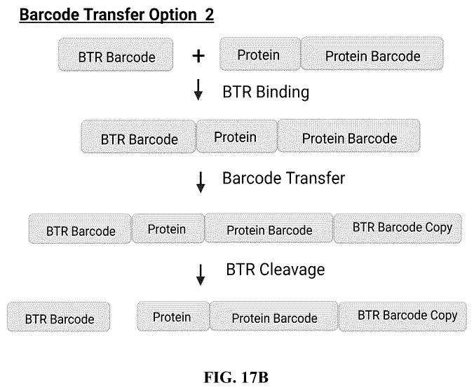

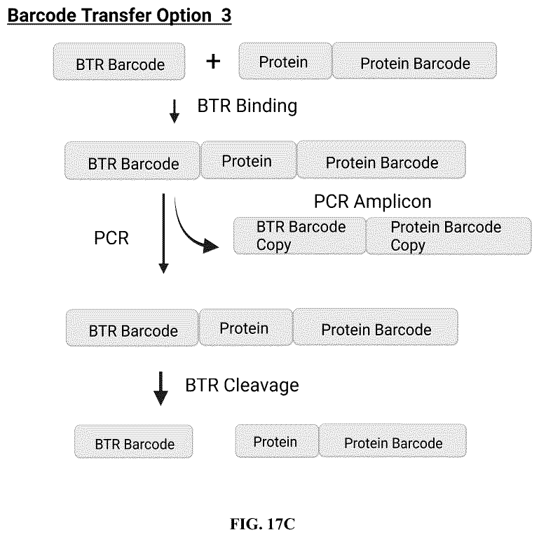

Multiple approaches to transfer of the peptide barcode to the BTR, or from the BTR to the peptide barcode are possible. FIGS. 17A-17C show several barcode transfer and cleavage schemes. In FIG. 17A, a BTR is contacted with a protein coupled to a protein barcode. The BTR tethers or couples to one end of the protein, and barcode transfer yields a copy of the protein barcode on another region (e.g., opposite end) of the BTR (the protein barcode is transferred or copied to the BTR). Cleavage of the BTR from the peptide may be performed (e.g., cleavage of the BTR-AC). FIG. 17B shows a similar approach, but the BTR barcode is copied to the protein barcode. FIG. 17C shows another example approach for barcode transfer. A BTR is contacted with a protein coupled to a protein barcode. The BTR tethers or couples to one end of the protein, and amplification is performed to generate a copy of the protein barcode coupled to a copy of the BTR barcode. Cleavage of the BTR from the peptide may be performed (e.g., cleavage of the BTR-AC).

Iteration: In some instances, one or more of the operations described herein may be iterated or repeated. Iteration of the operations may allow for sequential processing, analysis, or identification of the individual amino acids of the peptide, which can allow for reconstruction of the entire peptide. For example, referring to FIG. 1, the workflow may be performed to transfer the peptide barcode (or portion thereof) to the BTR-AC prior to cleavage of the BTR-AC from the peptide. The workflow may then be repeated to encode the identities of the n-1 terminal amino acid, the n-2 terminal amino acid, the n-3 terminal amino acid, etc., until the entire or portion of the peptide is sequentially removed. Each cleaved BTR-AC from a cycle may be collected, optionally pooled with other BTR-ACs, and analyzed, e.g., via nanopores, contacting with binding agents, nucleic acid sequencing, as described elsewhere herein. The readout of the BTR-AC nucleic acid barcode molecules may yield information on the cycle or order in which an amino acid occurs in a peptide (e.g., the NTAA, the n-1 NTAA, the n-2 NTAA, etc.) and the originating peptide, and the contacting with specific binding agents may yield information on the identity of the amino acid. Alternatively, or in addition to, the binding agents may comprise binding agent-identifying barcode molecules that can be transferred to the BTR-ACs for later detection (e.g., using DNA sequencing).

FIGS. 2, and 21-23 schematically show transfer of a nucleic acid barcode molecule of a binding agent to a BTR-AC. In FIG. 2, a BTR-AC comprising multiplexed barcode information (e.g., a peptide barcode and cycle information) is cleaved from the peptide (not shown) and contacted with a binding agent comprising a nucleic acid barcode molecule that identifies the binding agent. In some instances, the binding agent is coupled to a substrate, such as a bead, and the nucleic acid barcode molecule may be coupled directly to the binding agent (not shown) or to the substrate. The binding agent recognizes and binds to the amino acid portion of the BTR-AC. The nucleic acid barcode molecule of the binding agent may comprise a primer sequence that can anneal to a primer sequence of the BTR and optionally, identifying barcode sequence of the binding agent. An extension reaction, e.g., using a polymerase, may be performed to transfer the information from the binding agent nucleic acid barcode molecule to the BTR or from the BTR to the binding agent nucleic acid barcode molecule, thereby generating an extended barcode molecule. Subsequent to information transfer, the extended barcode molecule may be denatured and removed from the bead or from the BTR-AC. Alternatively, the extended barcode molecule or a strand of the extended barcode molecule may be directly analyzed using a sequencing system, e.g., nanopores or DNA sequencing system. The process may be iterated one or more times, which may yield additional copies of the extended barcode molecule and improve sensitivity or accuracy of the detection of the amino acid and encoded information in the extended barcode molecule. Alternatively or in addition to, the extended barcode molecule may be amplified to generate multiple copies.

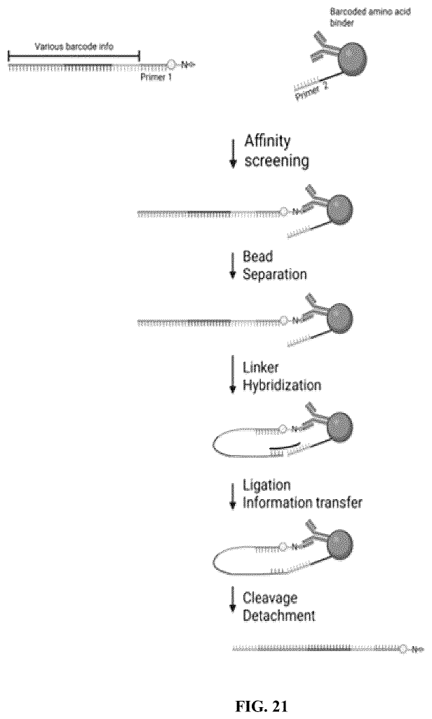

FIG. 21 schematically shows another example of transfer of a nucleic acid barcode molecule of a binding agent to the BTR-AC. In FIG. 21, a BTR-AC comprising multiplexed barcode information (e.g., a peptide barcode, a UMI, cycle information) is cleaved from the peptide (not shown) and contacted with a binding agent that is coupled (e.g., directly coupled or coupled via a substrate) to a nucleic acid barcode molecule that identifies the binding agent. The binding agent recognizes and binds to the amino acid portion of the BTR-AC. In some instances, the substrates may be separated after the binding, e.g., sorted by the binder type or amino acid that the binder recognizes (e.g., each amino acid type may be separated into different compartments to be separately processed or analyzed).

The nucleic acid barcode molecule of the binding agent may comprise a primer sequence that can be coupled to a primer sequence of the BTR using a splint or bridge oligo and optional ligation to generate a ligated barcoded molecule. The nucleic acid barcode molecule of the binding agent may also comprise identifying information of the binding agent or the binding partner. An extension reaction, e.g., using a polymerase, may optionally be performed. The ligated barcoded molecule may then be cleaved or detached from the binding agent (or substrate). The cleaved, ligated barcoded molecule may be optionally amplified and directly analyzed using a sequencing system, e.g., nanopores or DNA sequencing system.

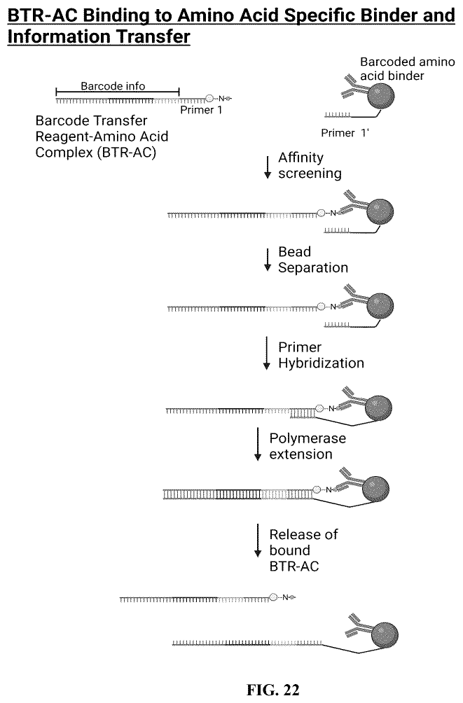

FIG. 22 schematically shows another example of transfer of a nucleic acid barcode molecule of a binding agent to the BTR-AC. In FIG. 21, a BTR-AC comprising multiplexed barcode information (e.g., a peptide barcode, a UMI, cycle information) is cleaved from the peptide (not shown) and contacted with a binding agent that is coupled (e.g., directly coupled or coupled via a substrate) to a nucleic acid barcode molecule that identifies the binding agent. The binding agent recognizes and binds to the amino acid portion of the BTR-AC. In some instances, the substrates may be separated after the binding, e.g., sorted by the binder type or amino acid that the binder recognizes (e.g., each amino acid type may be separated into different compartments to be separately processed or analyzed).

The nucleic acid barcode molecule of the binding agent may comprise a primer sequence that can anneal to a primer sequence of the BTR and optionally, identifying information of the binding agent or the binding partner of the binding agent. An extension reaction, e.g., using a polymerase, may be performed to transfer the information from the binding agent nucleic acid barcode molecule to the BTR or from the BTR to the binding agent nucleic acid barcode molecule, thereby generating an extended barcode molecule. Subsequent to information transfer, the extended barcode molecule may be denatured and removed from the bead or from the BTR-AC. In some instances, the binding agent may be unbound or dissociated from the amino acid portion of the BTR-AC. The liberated BTR-AC, or the extended copy that remains on the substrate may be analyzed, e.g., via DNA sequencing or nanopores.

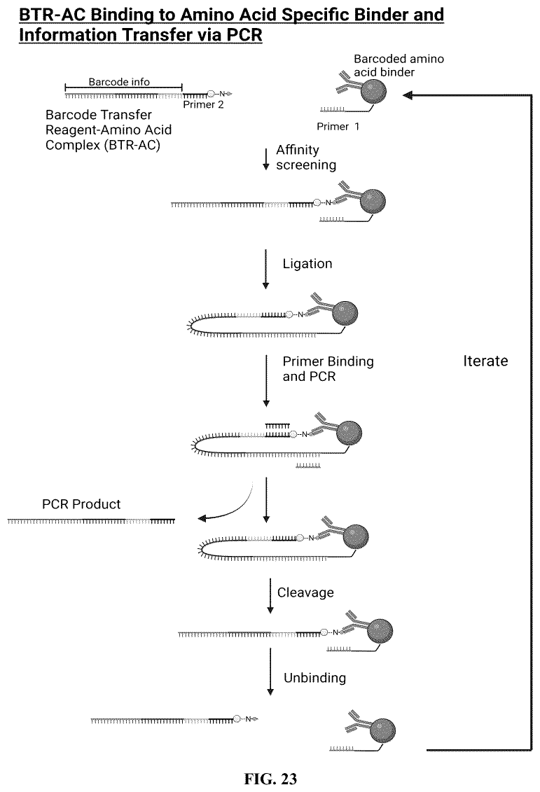

FIG. 23 schematically shows another example of transfer of a nucleic acid barcode molecule of a binding agent to the BTR-AC. In FIG. 23, a BTR-AC comprising multiplexed barcode information (e.g., a peptide barcode, a UMI, cycle information) is cleaved from the peptide (not shown) and contacted with a binding agent that is coupled (e.g., directly coupled or coupled via a substrate) to a nucleic acid barcode molecule that comprises a primer sequence and optionally, a sequence that identifies the binding agent. The binding agent recognizes and binds to the amino acid portion of the BTR-AC. In some instances, the substrates may be separated after the binding, e.g., sorted by the binder type or amino acid that the binder recognizes (e.g., each amino acid type may be separated into different compartments to be separately processed or analyzed).

The nucleic acid barcode molecule of the binding agent may comprise a primer sequence that can be coupled and ligated to a primer sequence of the BTR to generate a ligated barcoded molecule. Optionally, an extension reaction, e.g., using a polymerase, may be performed. The primer sequences of the binding agent and BTR can be used as priming sites for amplification to generate copies of the ligated barcoded molecule. Subsequent to information transfer and optional amplification, the ligated barcoded molecule may be cleaved (e.g., using an endonuclease) and optionally removed from the bead. In some instances, the binding agent may be unbound or dissociated from the amino acid portion of the BTR-AC. The liberated BTR-AC may be further analyzed, e.g., via DNA sequencing or nanopores. Any of the operations may be iterated or repeated; for example, multiple cycles of binding and information transfer can be conducted to get multiple reads of the amino acid, which may increase the signal-to-noise ratio. As illustrated, barcode round or cycle information can be present, but is not necessary.

Referring to FIGS. 2 and 21-23, subsequent to transfer or copying of the nucleic acid barcode molecule of the binding agent to the BTR-ACs to generate a coupled nucleic acid molecule (e.g., an extended barcoded molecule or a ligated barcoded molecule), the coupled nucleic acid molecule may comprise multiplexed information. For example, the coupled nucleic acid molecule may comprise cycle number (e.g., information on the sequence, order, or position that a given amino acid is located), primer sequences, peptide barcodes (e.g., to associate multiple amino acids as arising from a common peptide), and, in some instances, the binding agent barcodes (e.g., which identify the amino acid residue), which may all be used to reconstruct the peptide sequence.

It will be appreciated that while single instances of barcode transfer are depicted in FIGS. 2, and 21-23, that many reactions may be performed simultaneously and in parallel. For example, a plurality of BTR-ACs may be generated from a single or multiple peptides, and barcode transfer from the peptide barcodes to the plurality of BTR-ACs may be performed. Multiple iterations of the cleaving and barcode transfer may be performed to generate a pooled population of BTR-ACs, which may comprise different cycle numbers, peptide barcodes (if arising from different peptides), UMIs, etc. Affinity screening may be performed by providing a library of binding agents, each comprising identifying barcodes, that can recognize and specifically or partially specifically bind to individual amino acids of the cleaved BTR-ACs. The barcodes of the library of binding agents may be transferred to the BTR-ACs, as described above. Accordingly, subsequent to affinity screening, the BTR-ACs or derivatives thereof (e.g., complements, reverse complements, amplicons, etc.) may comprise multiplexed information including, in some examples, (i) cycle number, (ii) peptide barcode, (iii) binding agent (and hence, amino acid identity) barcode, each of which can be used to reconstruct the sequence of a peptide or plurality of peptides.

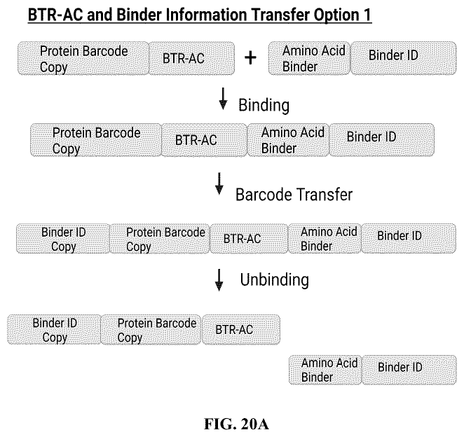

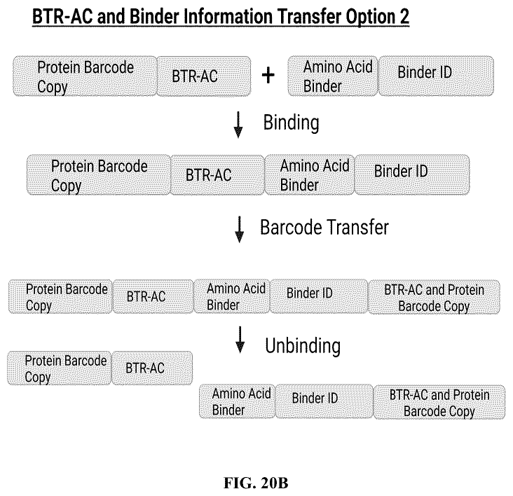

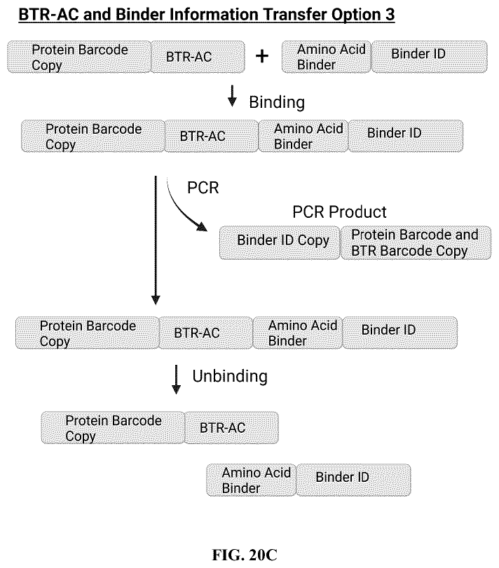

FIGS. 20A-20C schematically depict example workflows for transfer or copying of the binding agent barcode to the BTR barcode or from the BTR barcode to the binding agent. FIG. 20A shows copying or transfer of the binding agent barcode to the BTR barcode. A BTR-AC comprising a protein barcode (e.g., as shown as an output of the process of FIG. 17A) is contacted with a binding agent that is specific to one or more amino acids. The binding agent comprises a binding agent barcode that identifies the binding agent (e.g., the amino acid(s) that the binding agent binds). The barcode of the binding agent is transferred to the barcode of the BTR-AC, thereby yielding a multiplexed barcode comprising: the BTR barcode, the peptide barcode, and the binding agent barcode. The binding agent may then be removed from the BTR-AC. In FIG. 20B, the BTR-AC barcode (comprising the BTR barcode and the peptide barcode) is copied to the binding agent barcode. FIG. 20C shows another example approach for barcode transfer. A BTR-AC comprising a peptide barcode is contacted with a binding agent comprising a binding agent barcode. Amplification is performed to generate a copy of the binding agent barcode coupled to the BTR-AC barcode comprising the peptide barcode. Unbinding of the BTR-AC from the binding agent may be performed.

Applications: Beneficially, the methods and systems presented herein provide distinct advantages over current approaches to protein or peptide sequencing and has applications in diagnosing a disease, disorder, or condition. For example, in some cases, the systems and methods provided herein may be used to identify disease markers. In some cases, the systems and methods described herein may provide a diagnosis based on the spatial information and amino-acid sequence of proteins identified in a sample. In some cases, disease progression can be measured using the spatial information and amino-acid sequence of proteins identified using the systems and methods described herein. The methods and systems provided herein may also be useful in diagnosing diseases in clinical settings. For many diseases, patient samples such as saliva, blood serum, or cerebral-spinal fluid are used to identify protein markers associated with diseases. Some of these markers are low quantity requiring the need for a sensitive method to detect proteins. In some embodiments, the present disclosure may allow for analysis of disease markers from extracted protein samples taken from patients. In some embodiments, the system described herein may be housed in a device which will directly take patient samples for internal processing and analysis.

Additional advantages of the methods and systems disclosed herein include the ability to monitor the temporal dynamics of proteins. For example, cells passively release proteins or vesicles (e.g., via exocytosis or similar pathways) that contain protein cargo, which can be used to infer the protein expression levels of these cells through protein sequencing. The present disclosure provides for systems and methods that can allow for the continuous monitoring of cellular protein levels to diagnose a patient with a disease, monitor a patient's response to treatment, or monitor progression of a patient's disorder. In addition, the present disclosure can allow for the continuous monitoring of proteomic profile of a cell not in the context of diseases. In some embodiments, the present disclosure may allow for the analysis of protein expression levels from cells at multiple time points. In some cases, cells isolated from a patient at different time points can be analyzed to determine the changing proteomic profile. In some cases, the intracellular content of the cell can be obtained for testing. In some cases, the protein sequencing can include identification of post-translational modifications on proteins within the cell which can be used to determine the activation/functional state of the proteins. In addition to monitoring the temporal dynamics of protein expression in a cell, the present disclosure can allow for the tracking of protein movement within a cell or interactions between proteins within a cell.

The methods and systems described herein can be part of a service or device to be used to determine the immune profile of a patient. In some embodiments, a biological sample from the patient can be sorted to isolate a specific type of immune cell. Non-limiting examples of specific types of immune cells include B cells, T cells, macrophages, NK cells, lymphocytes, dendritic cells, neutrophils, or monocytes. In some cases, the specific type of immune cell may be sorted using fluorescence-activated cell sorting (FACS). In some embodiments, the protein content from the specific immune cell may be extracted and sequenced to determine the immune profile for that specific type of immune cell. In some embodiments, antibodies or receptors of interest can be separated from the extracted protein content from the cell using affinity reagents (e.g., Protein A, Protein G) and these proteins can be sequenced separately.

Systems and methods described herein offer promise in pharmaceutical research. For example, the presently disclosed systems and methods may aid in identifying candidate protein biomarkers as drug targets. Systems and methods described herein may also assist in pharmaceutical research directed towards approaches that study proteomic changes that result from the administration of candidate drugs. In one scenario, the presently disclosed system can be deployed as a service where pharmaceutical research samples are processed in a lab setting to identify target proteins. Alternatively, the system can be incorporated in a commercial device to be used in pharmaceutical research for the discovery of therapeutic protein targets. In addition, this disclosure can also be used to assay for protein-based biologics used in therapies.

The systems and methods described herein can be combined with single-cell processing methods (e.g., droplet microfluidics, microwells) that allow single-cell based proteomic studies, as described elsewhere herein. Similarly, the presently disclosed system may be incorporated with techniques for acquiring spatial information of proteins to yield novel technologies and products for spatial proteomics. Common approach for preserving the spatial information of tissues include spatially-barcoded DNA microarrays and hydrogel-based molecular retention methods. This system may be combined with such upstream tissue processing techniques to enable spatial proteomics.

The methods and systems provided herein may be used to identify the structure of a protein. In some embodiments, the methods and systems provided may be used to determine the folding of a protein. In some cases, the barcodes bind to the surface exposed amino acids on the protein, which allows for the identification of those residues once sequenced. In some embodiments, the methods and systems provided may be used determine the proximity of amino acids within the protein.

The methods and systems provided herein may be used to determine protein interactions within a protein complex. For example, interacting proteins within a protein complex may be tagged with different barcodes. In some cases, the barcodes on these different proteins may be extended by copying the barcode on the protein's interacting partner. In some cases, the extended barcode may allow for identification of the interaction proteins after sequencing.

The methods and systems provided herein may be used to enhance the detectability of amino acid residues or post-translational modifications at single molecule resolution. Non-limiting examples of post-translational modification include phosphorylation, acetylation, methylation, formylation, glycosylation, or ubiquitination. In some cases, reactions specific to each type of amino acid side chain or post-translational modification may be used to add to or convert the post-translational modification to a stable or an inert chemical group. In some cases, detectable chemicals groups (e.g., fluorophores or haptens) can be added to the post-translational modification. In some examples, fluorophore-modified post-translational modifications can be detected using single molecule imaging (e.g., total internal reflection, confocal, fluorescence, or wide-field microscopy). In some cases, the post-translational modification may be modified with bulky chemical groups or charge chemical groups to enable them to be detected in nanopore-based detection and protein sequencing approaches. In some cases, the post-translational modifications can be modified into chemical groups (e.g., biotin, digoxigenin) that allow for detection with binding agents (e.g., binding agents). In some examples, the binding agents can recognize the added chemical modification. In some examples, the binding agents can recognize the modified post-translational modification along with the attached amino acid.

The present disclosure may also be useful in molecular target profiling, e.g., to determine where a molecule (e.g., small molecule or biomolecule) binds to a protein. For example, molecules that bind to a protein may provide protective qualities to the target site of binding, which may prevent further downstream binding events (e.g., via binding agents, proteases, enzymes). In one such example, a molecule may bind to a protein and inhibit (e.g., via steric hindrance, electrostatic repulsion, etc.) interaction or binding of a protease, and thus prevent cleavage where the molecule is bound. Proteins or peptides that are not treated with the molecule may accordingly have different cleavage patterns when treated with a protease as compared to the treated condition. The methods and systems provided herein may be used to determine the location on a protein or peptide where a molecule binds. In one example, determining the location of a peptide or protein onto which a particular molecule of interest binds comprises allowing the molecule of interest to mix with a protein target to form a complex, and exposing the complex to a protease. The fragments of the protease-digested complex can be compared to the protein target without the molecule present and also digested with the protease (e.g., a control case). The protease-digested fragments from the test and control case can be labeled with separate multiplexing barcode tags and can then be sequenced with single molecule protein sequencing. The differences in cut sites can be determined and compared between both cases. Differences between these two conditions may be informative of the binding interaction the molecule of interest with the protein target or of the molecule of interest with the protease.

In protein engineering, proteins with novel or desired functions are selected from DNA sequences encoding variants or libraries of proteins. Current approaches to engineering new proteins, such as mRNA display, ribosome display, phage display, and monoclonal antibody production, require the physical linkage of phenotype (e.g., protein function) to genotype (e.g., the encoding DNA sequence) so that the function of a protein can be associated to its encoding DNA sequence. The methods and systems provided herein may be used to integrate single molecule protein sequencing approaches with directed evolution methods to enable protein sequences to be determined without requiring encoding DNA sequences to be associated with their respective proteins.

In some embodiments, proteins may be encoded and expressed from a library of encoding DNA or RNA sequences. In some cases, the proteins are sequenced using the herein disclosed methods and systems following some analysis of the function of these proteins (e.g., affinity, enzymatic activity, fluorescence). In some cases, once sequenced, additional round or cycles of mutagenesis and selection can be carried out from their respective encoding DNA sequences. In some examples, a library of proteins may be generated from a single encoding DNA or RNA sequence by performing mutagenesis (e.g., via introducing substitutions during translation). The library of proteins may then be barcoded, as described above, and sequenced, e.g., using the barcode transfer reagents, cleaving, contacting the BTR-ACs with a binding agent, etc.

In some embodiments, a library of proteins can be generated from a single encoding DNA or RNA sequence through introducing substitutions during translation. Non-limiting examples of how mutagenesis can be achieved include introducing tRNA molecules charged with different or missense amino acids or by altering the conditions (e.g., buffer composition) of prokaryotic or eukaryotic based ribosome translation to introduce errors during translation. In some cases, proteins generated in such a manner can be tagged with barcodes for identification and analyzed via protein sequencing, as described herein.

While nanopore-based technologies are under study for single-molecule proteomics, their accuracy is hampered by the sequence complexity of peptides and proteins. The ability of this technology to sequentially isolate amino acids with barcodes may enable accurate nanopore-based proteomic technologies. For example, the final amino acid identification and DNA sequence readout steps of this disclosure can be carried out using nanopore readers. This combination can yield new nanopore-based products that incorporate this disclosure for single molecule protein sequencing. Further, the present systems and methods may be incorporated into systems and methods for protein engineering.

### Substrate Conjugation

The present disclosure provides methods for coupling molecules (e.g., biomolecules such as nucleic acid molecules, peptides, lipids, carbohydrates, etc.) to a substrate. The substrate may be functionalized to allow for covalent or noncovalent coupling of the molecules to a substrate. The substrate may comprise any useful functional moiety, e.g., a reactive moiety, that can couple or conjugate to a molecule. In a non-limiting example, a reactive moiety may comprise a click chemistry moiety, such as an azide, alkyne, nitrone, alkene (e.g., a strained alkene), tetrazine, methyltetrazine, triazole, tetrazole, phosphite, phosphine, etc. A click chemistry moiety may be reactive in copper-catalyzed Huisgen cycloaddition or the 1,3-dipolar cycloaddition between an azide and a terminal alkyne, a Diels-Alder reaction (e.g., a cycloaddition between a diene and a dienophile), or a nucleophilic substitution reaction in which one of the reactive species is an epoxy or aziridine. A molecule that is to be coupled to a substrate may comprise a complementary click chemistry moiety to that of the substrate; for example, the substrate may comprise an alkyne moiety and the molecule to be coupled may comprise an azide moiety, which can react with the alkyne moiety of the substrate to generate a covalent linkage. In one such example, the substrate may comprise dibenzocyclooctyne (DBCO) moieties to which azide-comprising molecules (e.g., azide-DNA, azide-polymers, azide-peptides) can react and conjugate.

Alternatively, or in addition to, the reactive moiety may comprise a photoreactive moiety that may be activated when exposed to a photostimulus (e.g., light such as UV or visible light). Examples of photoreactive moieties include aryl (phenyl) azides (e.g., phenyl azide, ortho-hydroxyphenyl azide, meta-hydroxyphenyl azide, tetrafluorophenyl azide, ortho-nitrophenyl azide, meta-nitrophenyl azide), diazirines, azido-methyl-coumarins, benzophenones, anthraquinones, diazo compounds, diazirines, psoralen, and analogs or derivatives thereof.

The reactive moiety may comprise a carboxyl-reactive crosslinker group, such as diazomethane, diazoacetyl, carbonyldiimidazole, carbodiimides (e.g., 1-ethyl-3-(3-dimethylaminopropyl)carbodiimide hydrochloride (EDC)), dicyclohexylcarbodiimide (DCC)), or an amine-reactive group (e.g., N-hydroxysulfosuccinimide (NHS), Sulfo-NHS, or NHS-esters). The reactive group may comprise a crosslinking agent, which may comprise an NHS group, an EDC group, a maleimide, a thiol, a cystamine, an aldehyde, a succinimidyl group, an expoxide, an acrylate. Examples of crosslinking agents include, for example, NHS (N-hydroxysuccinimide); sulfo-NHS (N-hydroxysulfosuccinimide); EDC (1-Ethyl-3-[3-dimethylaminopropyl]); carbodiimide hydrochloride; SMCC (succinimidyl 4-(N-maleimidomethyl)cyclohexane-1-carboxylate); sulfo-SMCC; DSS (disuccinimidyl suberate); DSG (disuccinimidyl glutarate); DFDNB (1,5-difluoro-2,4-dinitrobenzene); BS3 (bis(sulfosuccinimidyl)suberate); TSAT (tris-(succinimidyl)aminotriacetate); BS(PEG)5 (PEGylated bis(sulfosuccinimidyl)suberate); BS(PEG)9 (PEGylated bis(sulfosuccinimidyl)suberate); DSP(dithiobis(succinimidyl propionate)); DTSSP (3,3′-dithiobis(sulfosuccinimidyl propionate)); DST(disuccinimidyl tartrate); BSOCOES (bis(2-(succinimidooxycarbonyloxy)ethyl)sulfone); EGS (ethylene glycol bis(succinimidyl succinate)); DMA (dimethyl adipimidate); DMP (dimethyl pimelimidate); DMS (dimethyl suberimidate); DTBP (Wang and Richard's Reagent); BM(PEG)2 (1,8-bismaleimido-diethyleneglycol); BM(PEG)3 (1,11-bismaleimido-triethyleneglycol); BMB (1,4-bismaleimidobutane); DTME (dithiobismaleimidoethane); BMH (bismaleimidohexane); BMOE (bismaleimidoethane); TMEA (tris(2-maleimidoethyl)amine); SPDP (succinimidyl 3-(2-pyridyldithio)propionate); SMCC (Succinimidyl trans-4-(maleimidylmethyl)cyclohexane-1-Carboxylate); SIA (succinimidyl iodoacetate); SBAP (succinimidyl 3-(bromoacetamido)propionate); STAB (succinimidyl(4-iodoacetyl)aminobenzoate); Sulfo-SIAB (sulfosuccinimidyl(4-iodoacetyl)aminobenzoate); AMAS (N-α-maleimidoacet-oxysuccinimide ester); BMPS (N-β-maleimidopropyl-oxysuccinimide ester); GMBS (N-γ-maleimidobutyryl-oxysuccinimide ester); Sulfo-GMBS (N-γ-maleimidobutyryl-oxysulfosuccinimide ester); MBS (m-maleimidobenzoyl-N-hydroxysuccinimide ester); Sulfo-MBS (m-maleimidobenzoyl-N-hydroxysulfosuccinimide ester); SMCC (succinimidyl 4-(N-maleimidomethyl)cyclohexane-1-carboxylate); Sulfo-SMCC (sulfosuccinimidyl 4-(N-maleimidomethyl)cyclohexane-1-carboxylate); EMCS (N-ε-malemidocaproyl-oxysuccinimide ester); Sulfo-EMCS (N-ε-maleimidocaproyl-oxysulfosuccinimide ester); SMPB (succinimidyl 4-(p-maleimidophenyl)butyrate); Sulfo-SMPB (sulfosuccinimidyl 4-(N-maleimidophenyl)butyrate); SMPH (Succinimidyl 6-((beta-maleimidopropionamido)hexanoate)); LC-SMCC (succinimidyl 4-(N-maleimidomethyl)cyclohexane-1-carboxy-(6-amidocaproate)); Sulfo-KMUS (N-κ-maleimidoundecanoyl-oxysulfosuccinimide ester); SPDP (succinimidyl 3-(2-pyridyldithio)propionate); LC-SPDP (succinimidyl 6-(3(2-pyridyldithio)propionamido) hexanoate); LC-SPDP (succinimidyl 6-(3(2-pyridyldithio)propionamido)hexanoate); Sulfo-LC-SPDP (sulfosuccinimidyl 6-(3′-(2-pyridyldithio)propionamido)hexanoate); SMPT (4-succinimidyloxycarbonyl-alpha-methyl-α(2-pyridyldithio)toluene); PEG4-SPDP (PEGylated, long-chain SPDP crosslinker); PEG12-SPDP (PEGylated, long-chain SPDP crosslinker); SM(PEG)2 (PEGylated SMCC crosslinker); SM(PEG)4 (PEGylated SMCC crosslinker); SM(PEG)6 (PEGylated, long-chain SMCC crosslinker); SM(PEG)8 (PEGylated, long-chain SMCC crosslinker); SM(PEG)12 (PEGylated, long-chain SMCC crosslinker); SM(PEG)24 (PEGylated, long-chain SMCC crosslinker); BMPH (N-β-maleimidopropionic acid hydrazide); EMCH (N-ε-maleimidocaproic acid hydrazide); MPBH (4-(4-N-maleimidophenyl)butyric acid hydrazide); KMUH (N-x-maleimidoundecanoic acid hydrazide); PDPH (3-(2-pyridyldithio)propionyl hydrazide); ATFB-SE (4-Azido-2,3,5,6-Tetrafluorobenzoic Acid, Succinimidyl Ester); ANB-NOS (N-5-azido-2-nitrobenzoyloxysuccinimide); SDA (NHS-Diazirine) (succinimidyl 4,4′-azipentanoate); LC-SDA (NHS-LC-Diazirine) (succinimidyl 6-(4,4′-azipentanamido)hexanoate); SDAD (NHS-SS-Diazirine) (succinimidyl 2-((4,4′-azipentanamido)ethyl)-1,3′-dithiopropionate); Sulfo-SDA (Sulfo-NHS-Diazirine) (sulfosuccinimidyl 4,4′-azipentanoate); Sulfo-LC-SDA (Sulfo-NHS-LC-Diazirine) (sulfosuccinimidyl 6-(4,4′-azipentanamido)hexanoate); Sulfo-SDAD (Sulfo-NHS-SS-Diazirine) (sulfosuccinimidyl 2-((4,4′-azipentanamido)ethyl)-1,3′-dithiopropionate); SPB (succinimidyl-[4-(psoralen-8-yloxy)]-butyrate); Sulfo-SANPAH (sulfosuccinimidyl 6-(4′-azido-2′-nitrophenylamino)hexanoate); DCC (dicyclohexylcarbodiimide); EDC (1-ethyl-3-(3-dimethylaminopropyl)carbodiimide hydrochloride); gluteraldehyde; formaldehyde; and combinations or derivatives thereof.

Molecules may also be attached to substrates using linkers. The linkers can have any useful number of functional groups or reactive groups and may be uni-functional (having one functional group), bi-functional, tri-functional, quadri-functional, or comprise a greater number of functional groups. In some instances, a molecule (e.g., nucleic acid molecule, peptide, or polymer) may be attached to a substrate using a heterobifunctional linker. The heterobifunctional linker may comprise any useful functional group, as described herein. Non-limiting examples of heterobifunctional linkers include: p-Azidobenzyol hydrazide (ABH), N-5-Azido-2-nitrobenzoyloxysuccinimide (ANB-NOS), N-[4-(p-Azidosalicylamido)butyl]-3′-(2′-pyridyldithio) propionamide (APDP), p-Azidophenyl Glyoxal monohydrate (APG), Bis [B-(4-azidosalicylamido)ethyl]disulfide (BASED), Bis [2-(Succinimidooxycarbonyloxy)ethyl] Sulfone (BSOCOES), BMPS, 1,4-Di [3′-(2′-pyridyldithio)propionamido] Butane (DPDPB), Dithiobis(succinimidyl Propionate) (DSP), Disuccinimidyl Suberate (DSS), Discuccinimidyl Tartrate (DST), 3,3′-Dithiobis(sulfosuccinimidyl Propionate (DTSSP), EDC, Ethylene Glycol bis (succinimidyl succinate) (EGS), N-(E-maleimidocaproic acid hydrazide (EMCH), N-(E-maleimidocaproyloxy)-succinimide ester (EMCS), N-Maleimidobutyryloxysuccinimide ester (GMBS), Hydroxylamine-HCl, MAL-PEG-SCM, m-Maleimidobenzoyl-N-hydroxysuccinimide Ester (MBS), N-Hydroxysuccinimidyl-4-azidosalicylic acid (NHS-ASA), PDPH, N-Succinimidyl bromoacetate (SBA), SIA, Sulfo-SIA, Succinimidyl-4-(N-maleimidomethyl)cyclohexane-1-carboxylate (SMCC), Succinimidyl 4-(p-maleimidophenyl) Butyrate (SMPB), Succinimidyl-6-[β-maleimidopropionamido]hexanoate (SMPH), N-Succinimidyl 3-[2-pyridyldithio]-propionate (SPDP), Sulfo-LC-SPDP, N-(p-Maleimidophenyl isocyanate (PMPI), N-Succinimidyl(4-iodoacetyl)Aminobenzoate (SIAB), Sulfo-MBS, Sulfo-SANPAH, Sulfo-SMCC, Sulfo-DST, Sulfo-EMCS, Sulfo-GMBS, N-Hydroxysulfosuccinimidyl-4-azidobenzoate (Sulfo-HSAB), Sulfosuccinimidyl(4-azidophenyl)-1,3 dithio propionate (Sulfo-SADP), Sulfosuccinimidyl 2-(m-azido-o-nitrobenzamido)-ethyl-1,3′-dithio propionate (Sulfo-SAND), Sulfosuccinimidyl-2-(p-azidosalicylamido)ethyl-1,3-dithiopropionate (Sulfo SASD), Sulfo-SIAB, Sulfo-SMCC, Sulfo-SMPB, and the like.

More than one type of molecule may be coupled to the substrate. For example, a substrate may be coupled to nucleic acid molecules and peptides. Alternatively, a substrate may be coupled to only one type of molecule (e.g., only nucleic acid molecules, only peptides, only lipids, only carbohydrates, etc.). A substrate may be coupled to any useful combination of molecules, linkers, reactive moieties or functional groups, which may be coupled at any useful density, as described elsewhere herein. For example, a multifunctional linker may be used to attach both a nucleic acid barcode molecule and a peptide to the substrate. Alternatively, the substrate may comprise a plurality of bifunctional linkers that can conjugate to different molecules. In another example, a substrate may comprise a linker and reactive sites; the linker may be used to attach one type of molecule (e.g., peptides or nucleic acid molecules), whereas the reactive sites may be used to attach another type of molecule (e.g., nucleic acid molecules or peptides).

Linkers can comprise other functional portions, such as spacers (e.g., polymer chains, e.g., PEG, alkyl chains, etc.), cleavage sites (e.g., disulfide bridges that are cleavable upon application of a chemical stimulus, photocleavable or thermocleavable moieties, etc.), enzyme recognition sites, etc.

The proximity of a molecule coupled to a substrate to its nearest neighbor (e.g., another molecule) may be controlled using a variety of approaches, e.g., self-assembling monolayers, patterning approaches, linking moieties, etc. In some instances, it may be advantageous to have two molecules in close proximity (e.g., two polymerizable molecules, such as a peptide and a nucleic acid molecule, or two nucleic acid molecules). For instance, with respect to the sequencing approaches described herein, binding agents may be coupled to a binding agent barcode that identifies a particular amino acid or set of amino acids. The binding agent and the binding agent barcode may be coupled to a substrate, and more than one binding agent barcode may be present on the substrate. In some instances, the proximity of the molecules (e.g., binding agent barcodes) may be mediated using tethering molecules, such as nucleic acid molecule “staples” or multi-functional linkers.

Nucleic acid molecules may be coupled to a substrate by direct coupling. In such instances, the substrate or the nucleic acid molecules may comprise functional moieties that can interact. For example, the substrate and nucleic acid molecules may comprise a complementary click chemistry pair, e.g., alkyne and azide. In one such example, a substrate may comprise alkyne moieties (e.g., DBCO), which can be reacted with azide-functionalized nucleic acid molecules. The nucleic acid molecules may be reacted with the alkyne moieties in a click chemistry reaction to covalently link the substrate to the nucleic acid molecules. In another example, the substrate may comprise avidin or streptavidin moieties, to which biotinylated nucleic acid molecules may interact and bind non-covalently.

Alternatively, or in addition to, the nucleic acid molecules may be coupled to a substrate using a linker, e.g., as described elsewhere herein. The linker may comprise at least two functional groups (e.g., a heterobifunctional linker) that can couple to both the substrate and the nucleic acid molecules. In an example, the substrate may comprise an amine group, and alkyne-functionalized DNA primers (e.g., DBCO-DNA primers) may be attached using a linker such as azidoacetic acid NHS ester. In another example, amine-functionalized substrates may be coupled to azide-functionalized DNA primers using a DBCO-NHS ester or DBCO-PEG-NHS ester linker. As described elsewhere herein, the linkers may comprise additional functional moieties (e.g., cleavage sites, spacers such as polymer or alkyl chains).

Similarly, peptides may be coupled to a substrate by direct coupling or by using a linker. A peptide may be coupled to a substrate at a terminus of the peptide (e.g., C terminus or N terminus), at an internal residue or amino acid of the peptide, or at multiple locations along the peptide. In examples of direct coupling, a peptide may be functionalized with a moiety that can interact with a moiety of the substrate (e.g., click chemistry pair, avidin-biotin). For example, the substrate and peptides may comprise a complementary click chemistry pair, e.g., alkyne and azide, or binding partners such as avidin and biotin. In one example of a click chemistry pair, a substrate may comprise alkyne moieties (e.g., DBCO), which can be reacted with azide-functionalized peptides. The peptides may be reacted with the alkyne moieties in a click chemistry reaction to covalently link the substrate to the peptides. In another example, the substrate may comprise avidin or streptavidin moieties, to which biotinylated peptides may interact and bind non-covalently.

Alternatively, or in addition to, the peptides may be coupled to a substrate using a linker, e.g., as described elsewhere herein. The linker may comprise at least two functional groups (e.g., a heterobifunctional linker) that can couple to both the substrate and the nucleic acid molecules. In an example, the substrate may comprise an amine group, and alkyne-functionalized peptides may be attached using a linker such as azidoacetic acid NHS ester. In another example, amine-functionalized substrates may be coupled to azide-functionalized peptides using a DBCO-NHS ester or DBCO-PEG-NHS ester linker. In yet another example, substrates comprising an amine group may be coupled to an azide-functionalized peptide using EDC and Sulfo-NHS.

A peptide may be functionalized with a functional moiety to enable attachment or coupling of the peptide to the substrate. The functional moiety may comprise a click chemistry moiety or other linking moiety and can be attached to the peptide at a peptide terminus (N-terminus or C-terminus), or at an internal amino acid. Chemical approaches to functionalize peptides can include C-terminal-specific conjugation (e.g., via C-terminal decarboxylative alkylation) using photoredox catalysis, e.g., as described by Bloom et al, Nature Chemistry 10, 205-211. 2018. and Zhang et al, ACS Chem. Biol. 2021, 16, 11, 2595-2603, each of which is incorporated by reference herein in its entirety, or amide coupling to an amine-functionalized surface. N-terminal attachment may comprise amide coupling of the N-terminus amine group to a carboxylic group functionalized surface or using 2-pyridinecarboxaldehyde variants. Alternatively, or in addition to, functionalization of terminal ends of peptides may be achieved enzymatically, e.g., using carboxypeptidases or amidases for C-terminal functionalization (e.g., as described in Xu et al, ACS Chem Biol. 2011 Oct. 21; 6(10): 1015-1020; Zhu et al, Chinese Chemical Letters. 2018, Vol 29 Issue 7, Pages 1116-1118; and Zhu et al, ACS Catal. 2022, 12, 13, 8019-8026, each of which is incorporated by reference herein in its entirety), Sortase A, subtiligase, Butelase I, or trypsiligase. In some examples, ubiquitin ligase can be used to attach ubiquitin proteins with linker moieties to substrates. These linker moieties can then be used to chemically attach proteins to ubiquitin-coupled substrates. Internal amino acid residues may be coupled to substrates using, for example, amide coupling using EDC/NHS chemistry or DMT-MM to Glutamate or Aspartate residues, alkylation or disulfide bridge labeling of cysteines, or amide coupling to lysine residues.

A peptide may be treated prior to, during, or subsequent to coupling of the peptide to a substrate. In some examples, it may be advantageous to block or protect primary amines or carboxyl groups and optionally, de-block or de-protect the N-terminus primary amine or C-terminus carboxy group in order to facilitate attachment of the N-terminus or C-terminus to a substrate. In an example, single-point (e.g., C-terminal) selective attachment of peptides can be achieved by reacting the peptide with a linker comprising an amine-reactive group (e.g., isothiocyanates such as PITC) and a reactive group (e.g., click chemistry group). The linker can be, for example, PITC-conjugated click chemistry moieties such as PITC-azide, PITC-alkyne, optionally with spacer moieties in between, e.g., PITC-alkyl-azide, PITC-PEG-azide, PITC-alkyl-alkyne, PITC-PEG-azide. In some instances, the linker is the same molecule as the BTR. The linker may react with and “blocks” the primary amines (e.g., modifies lysines), including the N-terminus. Subsequent cleavage of the N-terminal amino acid (e.g., using an Edman reagent, such as acid), can be performed, and one of the remaining modified lysines may be attached to a substrate (e.g., using the click chemistry moiety coupled to the amine-reactive group). Optionally, the peptide may be treated with a protease, e.g., LysC, which cleaves peptides such that a remaining peptide has a C-terminal lysine and such that the remaining peptide comprises a primary amine only at the C-terminal lysine residue and the N-terminus; such a cleavage may be performed prior to reacting the amine-reactive group, e.g., as shown by Xie et al. Langmuir 2022, 38, 30, 9119-9128, which is incorporated by reference herein in its entirety.

Similarly, carboxylic groups can be reacted in a way to enable C-terminal or internal residue attachment. In an example of C-terminal conjugation, carboxyl groups may be labeled with a C-terminal sequencing reagent, such as isothiocyanate, when treated with an activating reagent (e.g., acetic anhydride) to generate a peptide-thiohydantoin (at the C-terminus) and “blocked” carboxyl groups on the aspartic acid and glutamic acid residues. The thiohydantoin may then be reacted to couple to a substrate. Alternatively, cleavage of the C-terminal amino acid via a single round of C-terminal sequencing degradation, or via a protease, exposes only a single reactive carboxylic group at the C-terminal amino acid. The single reactive C-terminal carboxylic group can then be used as a reactive moiety for a single attachment site.

In another approach, a peptide or protein can be attached via the N-terminus using the specific reactivities of the N-terminus amine group. Amine-based reactions, such as amide coupling, can be carried out at low pH where only the N-terminal amine group is active. In addition, 2-pyridinecarboxyaldehyde and variants can be used to react to the N-terminal amine group.

In some instances, a peptide may be conjugated to a substrate using a polymerization reaction, e.g., a free radical polymerization, such as using PEGylated peptides, methacrylamide-modified peptides, Michael-type addition of maleimide-terminated oligo-NIPAAM-conjugated peptides; photocrosslinking of azophenyl-conjugated peptides, or other polymerization reactions with monomer-conjugated peptides, e.g., as described by Krishna et al. Biopolymers. 2010; 94(1): 32-48, which is incorporated by reference herein in its entirety.

Multiple types of molecules may be attached to a substrate. The substrate may comprise, coupled thereto, any combination of molecules, including but not limited to peptides, proteins (e.g., enzymes, antibodies, nanobodies, antibody fragments), nucleic acid molecules, lipids, carbohydrates or sugars, metabolites, small molecules, polymers, metals, viral particles, biotin, avidin, streptavidin, neutravidin, etc. The multiple types of molecules may be attached simultaneously to the substrate or in a sequential manner. For example, a substrate may be treated to conjugate nucleic acid molecules and subsequently treated to conjugate peptides, or alternatively, the substrate may be treated to conjugate peptides prior to the nucleic acid molecules.

A substrate, or portion thereof, may be subjected to conditions sufficient to passivate the substrate or portion thereof. Passivation of a substrate may be useful for a variety of purposes, such as preventing nonspecific binding of binding agents, altering the surface density of a molecule (e.g., increasing the density of nucleic acid molecules or peptides), blocking reactive sites (e.g., blocking available click chemistry moieties subsequent to conjugation of the molecules on the substrate), etc. Passivation may be achieved using chemical approaches, e.g., deposition of blocking agents such as proteins (e.g., albumin), Tween-20, polymers, metals or metal oxides, or biochemical approaches, e.g., using metal microbes. Substrates comprising reactive moieties may also be passivated following molecule conjugation (e.g., coupling of nucleic acid molecules, peptides, etc.) by reacting any unreacted sites with an appropriate molecule. For example, a substrate comprising click chemistry moieties, e.g., DBCO beads, may be coupled to molecules of interest (e.g., such as nucleic acid molecules, peptides, binding agents) at a useful density using click chemistry (e.g., azide-nucleic acid molecules, azide-peptides). Unreacted sites may be passivated by providing and reacting complementary click-chemistry molecules, e.g., azide-polymers (e.g., PEG-azide), which may reduce downstream nonspecific interactions.

Substrate passivation may occur at any useful time or step. For instance, passivation to block unreacted DBCO sites may be performed prior to, during, or subsequent to conjugation of analytes or other molecules of interest (e.g., peptides and nucleic acid molecules). The passivation may be controlled by stoichiometry or densities of the passivating agent relative to the molecules of interest, or by physical approaches, e.g., photopatteming, self-assembling monolayers, etc.

### Sample Processing

The present disclosure also provides for methods of processing samples. One or more methods for processing samples may comprise preparation of biological samples for analysis, which, in some instances, includes partitioning of cells for conducting single-cell analysis. A method for processing a biological sample may comprise extraction or isolation of one or more peptides or proteins from the biological sample for further processing and analysis, as is described elsewhere herein.

Preparation of Cell Suspensions for Single-Cell Analysis: The methods described herein may involve preparation of single cell suspensions from a biological sample. Single cell suspensions may be prepared from biological samples by dissociating cells and optionally, culturing them in a liquid medium. In some instances, biological samples comprise a liquid sample. For example, a biological sample may comprise a bacterial liquid culture, a mammalian liquid culture, a blood, plasma, or serum sample. Processing of such liquid samples may include centrifugation (e.g., to isolate cells), resuspension of cells in a suitable medium, such as Dulbecco's Phosphate Buffered Saline (DPBS), and optional culturing of the isolated cells.

A biological sample may comprise cultured cells, e.g., cell cultured in suspension, or cells adhered to a solid surface, such as petri dishes or tissue culture dishes. Cultured adherent cells samples may be treated to generate a cell suspension, e.g., via a protease such as trypsin, to detach the cells from the surface. A biological sample may comprise a tissue or biopsy sample. A tissue or biopsy sample may be processed mechanically or enzymatically to generate a cell suspension. Such processing may include sonication (mechanical treatment) or enzymatic treatment, such as the use of pronase, collagenase, hyaluronidase, metalloproteinases, trypsin, or other enzymes that digest extracellular matrix components. The dissociated cells can then be stored in a suitable buffer, such as DPBS.

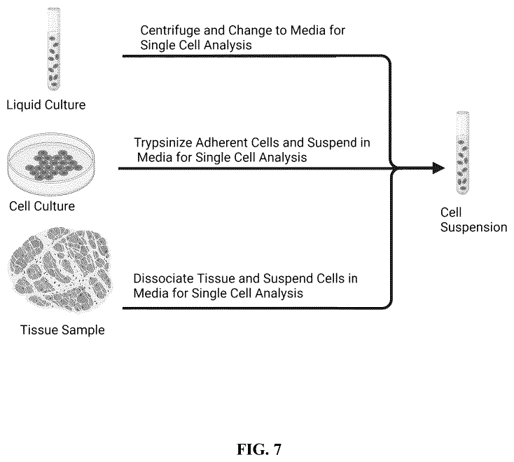

FIG. 7 schematically shows techniques for obtaining a cell suspension from different biological sample types. A liquid culture (e.g., from a biopsy, cultured cells, etc.) may be centrifuged to pellet and collect cells. Adherent cell cultures (e.g., from a tissue culture plate or dish) may be detached, e.g., using trypsin, and suspended in a media for further analysis or processing. A tissue sample may be processed to dissociate the tissue and harvest cells, which may optionally be cultured in media, prior to further analysis or processing.

Cell Sorting: A biological sample or a cell suspension may be subjected to sorting to isolate a cell of interest. Sorting may be performed to select or isolate a cell based on a quality or characteristic of the cell, e.g., expression of a protein target, size, deformability, fluorescence or other optical property, or other physical property of the cell. Sorting may be accomplished using any number of approaches, e.g., using immunosorting (e.g., fluorescence activated cell sorting (FACS) or magnetic activated cell sorting (MACS)), electrophoretic approaches, chromatography, microfluidic approaches (e.g., using inertial focusing, cell traps, electrophoresis), acoustic sorting, optical sorting (e.g., optoelectronic tweezers), mechanical cell picking (e.g., using manual or robotic pipettes) or passive approaches (e.g., gravitational settling).

Partitioning: Cells of a biological sample or cell suspension may be partitioned into individual partitions such that at least a subset of the individual partitions comprises a single cell. The individual partitions may comprise a barcode molecule (e.g., fluorophore or set of fluorophores, nucleic acid barcode molecules, etc.). Barcode molecules may be unique to the partition, such that each individual partition comprises a different barcode sequence than other partitions. The barcode molecules may be loaded into the individual partitions at any useful ratio of barcode molecules to sample species (e.g., cells, proteins, nucleic acid molecules). The barcode molecules may be loaded into partitions such that about 0.0001, 0.001, 0.1, 1, 2, 3, 4, 5, 6, 7, 8, 9, 10, 20, 50, 100, 500, 1000, 5000, 10000, or 200000 barcodes are loaded per sample species. In some cases, the barcodes are loaded into partitions such that more than about 0.0001, 0.001, 0.1, 1, 2, 3, 4, 5, 6, 7, 8, 9, 10, 20, 50, 100, 500, 1000, 5000, 10000, or 200000 barcodes are loaded per sample species. In some cases, the barcodes are loaded in the partitions so that less than about 0.0001, 0.001, 0.1, 1, 2, 3, 4, 5, 6, 7, 8, 9, 10, 20, 50, 100, 500, 1000, 5000, 10000, or 200000 barcodes are loaded per sample species.

A partition may assume any useful geometry such as a droplet, a microwell, a solid substrate, a gel (e.g., a cell encapsulated in a gel bead), a bead, a flask, a tube, a spot, a capsule, a channel, a chamber, or other compartment or vessel. A partition may be part of an array of partitions, e.g., a droplet in a microfluidic device, a microwell of a microwell plate, a spot on a multi-spot array, etc.

Lysis, Permeabilization, and Analyte Extraction: Single cells (e.g., in partitions) may be processed to obtain one or more analytes contained therein. A method for processing a single cell may comprise lysing the cell to release the contents into the individual compartment or partition. Lysis may be performed using a detergent (e.g., Triton-X 100, sodium dodecyl sulfate, sodium deoxycholate, CHAPS), RIPA buffer, a change in temperature (e.g., elevated or lower temperature, freezing, freeze-thawing), enzymes, mechanical lysis (e.g., sonication, application of mechanical force), electrical lysis, or a combination thereof. Lysis may be performed in the presence of protease inhibitors to prevent degradation or digestion of the proteins from the cell. The contents may optionally be further processed, e.g., subjected to purification or extraction, denaturation of proteins or peptides, enzyme or chemical digestion, etc. In some instances, the contents may be subjected to enzymatic digestion to remove nucleic acid molecules, e.g. using nucleases such as DNAse or RNAse. Alternatively or in addition to, a cell may be fixed (e.g., using a fixative) and/or permeabilized. Examples of fixatives include aldehydes (e.g., glutaraldehyde, formaldehyde, paraformaldehyde), alcohols (e.g., methanol, ethanol), acetone, acids (e.g., acetic acid, Davidson's AFA), oxidizing agents (e.g., osmium tetroxide, potassium dichromate, chromic acid, permanganate salts), Zenker's fixative, picrates, Hepes-glutamic acid buffer-mediated organic solvent protection effect (HOPE), or Karnovsky fixative. Cell permeabilization may be achieved mechanically (e.g., using sonication, electroporation, shearing) or chemically (e.g., using an organic solvent such as methanol or acetone or detergents such as saponin, Tween-20, Triton X-100).

Protein Processing: The biological sample (or single cell suspensions or partitioned cells) may be further processed to enable proteomic analysis. For example, de-aggregation of proteins in the sample may be performed, e.g., using chemical or mechanical approaches. Chemical de-aggregation methods can include but are not limited to sodium dodecyl (SDS), Triton-X 100, 3-((3-cholamidopropyl)dimethylamminio)-1-proppanesulfonate (CHAPS), ethylene carbonate, or formamide. Mechanical de-aggregation methods can include but are not limited to sonication or high temperature treatment. The biological sample (or single cell suspensions or partitioned cells) may be subjected to conditions sufficient to denature one or more proteins. Denaturation may be achieved using heat, chemicals (e.g., SDS, urea, guanidine), reducing agents (e.g., dithiothreitol (DTT), beta mercaptoethanol, TCEP), urea, enzymes (e.g., ClpX, ClpS, unfoldases). Other biological or chemical agents may be included during the protein processing, e.g., lysozymes, papain, cruzain, trypsin, protease inhibitors, nucleases or nuclease-containing proteins (e.g., DNAse, RNAse, DNA glycosylases, restriction endonucleases, transposases, micrococcal nucleases, Cas proteins).

Peptides or proteins may be fragmented prior to analysis. Fragmenting proteins may be useful in reducing the size of the proteins and allow for efficient processing of peptides, as is described elsewhere herein. Fragmentation may be performed using proteases, e.g., trypsin, chymotrypsin, pepsin, Lys-C, Glu-C, Proteinase K, furin, thrombin, endopeptidase, papain, subtilisin, elastase, enterokinase, genenanse, endoproteinase, metalloproteases, or with chemical treatment, e.g., cyanogen bromide, hydrazine, hydroxylamine, formic acid, BNPS-skatole, iodosobenzoic acid, 2-nitro-5-thiocyanobenzoic acid, etc. Alternatively or in addition to, fragmentation may be performed using mechanical methods, such as sonication, vortexing, mechanical stirring, using temperature changes (e.g., freeze/thaw, heating), or other fragmentation approach.

Enrichment of proteins or peptides in a biological sample may be performed, e.g., for separating proteins and peptides from cellular debris or other types of analytes (e.g., nucleic acids, lipids, carbohydrates, metabolites). Such enrichment may include, for example, the use of affinity columns (e.g., ion exchange), size exclusion columns, affinity precipitation (e.g., immunoprecipitation), chromatography (e.g., HPLC), or electrophoresis. In instances where cells are partitioned prior to enrichment, the enrichment may be performed using microbeads, affinity microcolumns, affinity beads, etc. In some instances, fractionation may be performed on the proteins or peptides, which may be used to separate the proteins by size, hydrophobicity, charge, affinity, size, mass, density, etc. In some instances, proteins or peptides from a cell or library may be sorted or enriched based on a functional characteristic (e.g., enzyme activity, binding to a molecule of interest, etc.). In such examples, a library of peptides may be screened to identify a peptide or protein of interest; the peptides or proteins of interest may be separated or isolated from the library and tagged with barcodes and sequenced, as described herein.

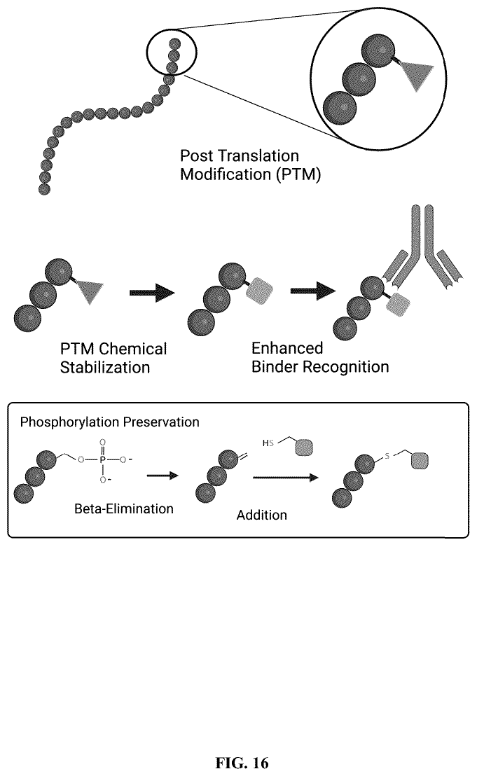

Proteins or peptides may be modified, e.g., to enable better detection (e.g., to improve binding of the binding agents), to protect or stabilize post translational modifications or residues that are sensitive to Edman degradation, or for any other useful purpose. FIG. 16 schematically shows an example of a post-translational modification that can be modified to improve or enhance the recognition and binding of a binding agent (e.g., antibody or antibody fragment) to the PTM. In one such example, a peptide may comprise a phosphorylated amino acid (naturally occurring PTM). The peptide may be subjected to a beta-elimination and then Michael addition of a thiol group, thereby generating a modified amino acid. The peptide may subsequently be subjected to conditions sufficient to tether the terminal amino acid or derivative thereof to a BTR to generate a BTR-AC, cleavage of the BTR-AC from the peptide, and contacting the BTR-AC with a binding agent. The binding agent may have improved specificity or affinity to the modified amino acid or PTM than to the native amino acid or PTM. In another example, cysteine residues may be alkylated, e.g., treated with iodoacetate or chloroacetate. In other examples, lysine chains may be blocked or protected using, for example, PITC. In other examples, epitope tags or affinity tags may be added to individual amino acids of a peptide, e.g., fluorescent tags, haptens, nucleic acids, lipids, sugars, tags, chemical moieties, proteins or peptides, somamers, etc. may be added to individual amino acids of a peptide.

Peptides may be barcoded, in bulk or in partitions. Peptides may be barcoded with any useful type of barcode molecule, e.g., spectral or fluorescent barcodes, mass tags, nucleic acid barcode molecules, etc. The barcode molecules may allow for identification of an originating peptide, a partition, a sample, a cell, or cell compartment. For example, a cell sample may be partitioned such that a partition comprises at most one cell; the partition may comprise a unique barcode molecule (e.g., nucleic acid barcode molecule) that identifies the partition and thus the cell. Subsequent labeling of the peptides within the partition (e.g., by permeabilizing or lysing the cell) with the barcode molecules may be useful in identifying the peptides as arising or originating from the same cell or partition. In other examples, a substrate may comprise nucleic acid molecules comprising a unique barcode sequence that differs from barcode sequences of other substrates. As such, the barcode sequence may be used to identify the substrate. In some instances, barcoded substrates may be partitioned with cell samples, such that at least a subset of the partitions comprise a single cell and a single barcoded substrate. As such, the peptides arising from the single cell and transferred to the barcoded substrate may all be identifiable as originating from the single cell. Barcode molecules may comprise additional useful functional sequences, e.g., UMIs, primer sites, restriction sites, cleavage sites, transposition sites, sequencing sites, read sites, etc.

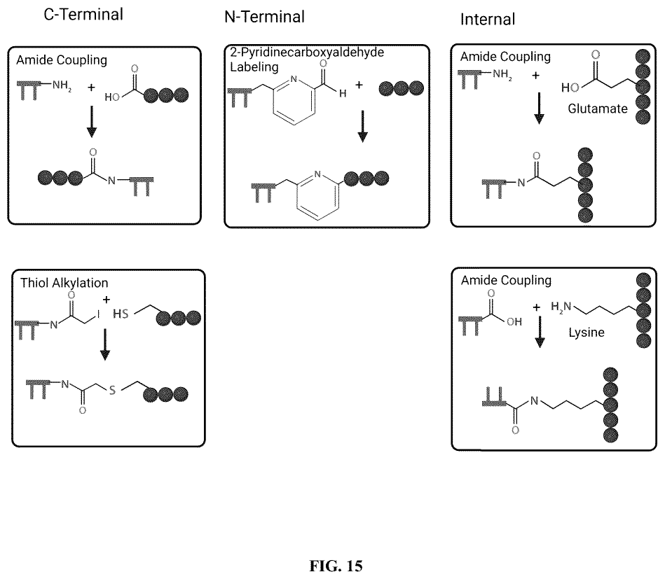

Attachment of barcode molecules to peptides may be achieved using any suitable chemistry. For example, C-terminal conjugation of nucleic acid barcode molecules may be achieved by amide coupling of amine-conjugated DNA barcode molecules to peptides or by thiol alkylation, e.g., reacting a thiolated peptide with an alkylated (e.g., iodoacetamide) DNA barcode molecule. N-terminal conjugation can be achieved, for instance, using 2-pyridinecarboxyaldehyde labeling of a DNA barcode and reacting with the N-terminus of a peptide. Internal residues, e.g., glutamate, can also be labeled with amine-conjugated DNA barcode molecules or carboxylated DNA barcodes (e.g., to react with primary amines in lysine). Examples of such conjugation approaches are schematically illustrated in FIG. 15.

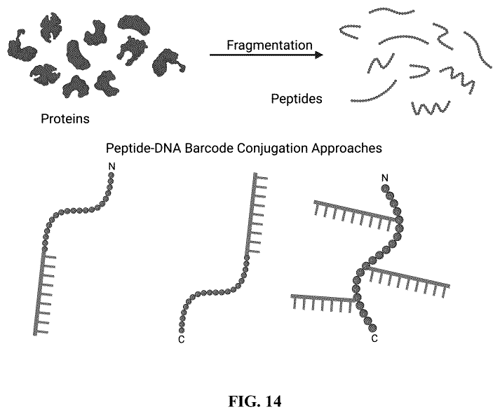

Individual peptides may be barcoded at multiple locations for a given peptide. A peptide may be labeled at multiple sites with the same or different barcode sequences. For example, a peptide may be partitioned into a partition comprising a plurality of identical barcode molecules that comprise a barcode sequence that is unique to the partition. The peptide may be labeled at a single or multiple sites with the unique partition barcode sequence, optionally each comprising a unique molecular identifier (UMI), such that subsequent downstream analysis (e.g., sequencing) may be attributable to the same peptide using the barcode sequence. In some instances, a terminus of the peptide (e.g., N-terminus or C-terminus) or an internal amino acid may be labeled with a barcode, as shown schematically in FIG. 14. In some instances, the peptide may be fragmented prior to analysis or sequencing; accordingly, upstream attachment of multiple identical barcode molecules to the same peptide may allow for attribution of the sequence analysis back to a single peptide. Barcoding of peptides may occur prior to, during, or subsequent to fragmentation. Peptides may be labeled with barcodes (e.g., nucleic acid barcode molecules) using any suitable chemistry, e.g., as described above, or using bifunctional or trifunctional linkers comprising multiple linking moieties, e.g., as described elsewhere herein, such as click chemistry moieties, NHS-esters, EDC, etc. For example, C-terminal attachment may comprise amide coupling to C-terminus carboxylic group or photoredox tagging of C-terminus carboxylic group (e.g., to add an electrophile tag). N-terminal attachment may comprise amide coupling to N-terminus amine group, where specific attachment can occur at low pH, or using 2-pyridinecarboxaldehyde variants for specific attachment to N-terminus. Internal attachment may comprise, for example, amide coupling using EDC/NHS chemistry or DMT-MM to Glutamate or Aspartate; alkylation or disulfide bridge labeling of cysteines; or amide coupling to lysine residues (see, e.g., FIG. 15).

In some examples, a peptide may be labeled with different barcode molecules, which can be indexed by proximity to one another, e.g., using primers that can anneal to adjacent barcode molecules. In one such approach, after a protein has been labeled with a plurality of barcodes with different barcode sequences, proximity-based polymerase extension may be used to copy and associate the sequence of adjacent barcodes. For example, each barcode molecule may comprise a primer binding site, to which a dual-primer linker sequence comprising two sequences is annealed. The dual primer linker sequence can bind to the primer binding sites of two adjacent barcodes. An extension reaction, e.g., using a polymerase, may extend and copy the barcode sequences of the adjacent barcodes. Subsequently, the dual primer linker sequence, which now has copies of the two adjacent barcodes, may be removed and sequenced. From the sequencing reads, an adjacency matrix of barcode sequences may be generated (e.g., to correspond barcode sequences on a single dual primer linker as spatially adjacent). Accordingly, each of the barcode sequences may be associated with a nearby adjacent barcode sequences, and as such, peptide portions may be aligned or attributed as being adjacent. Such an approach may be useful in instances where the peptide is fragmented, such that individual fragments of a peptide may be corresponded with the nearest neighbor using the barcode sequences, and, in some instances, traced back to the originating peptide or protein from which two fragments arise. For example, a peptide may be barcoded with a plurality of barcodes with different barcode sequences, and the dual primer linker sequence may be added to couple (e.g., via hybridization or via blunt-end ligation) to two barcodes located adjacent to one another (e.g., within 1, 2, 3, 4, 5, 6, 7, 8, 9, 10, or more amino acids). In some instances, the peptide may thereafter be fragmented. The dual primer linker sequence may be copied or amplified at any useful step or operation, e.g., prior to, during, or subsequent to peptide sequencing using a plurality of BTRs. Sequencing of the dual primer linker sequences may then associate or identify two adjacent barcode sequences as arising from the same peptide and as being located adjacent to one another.

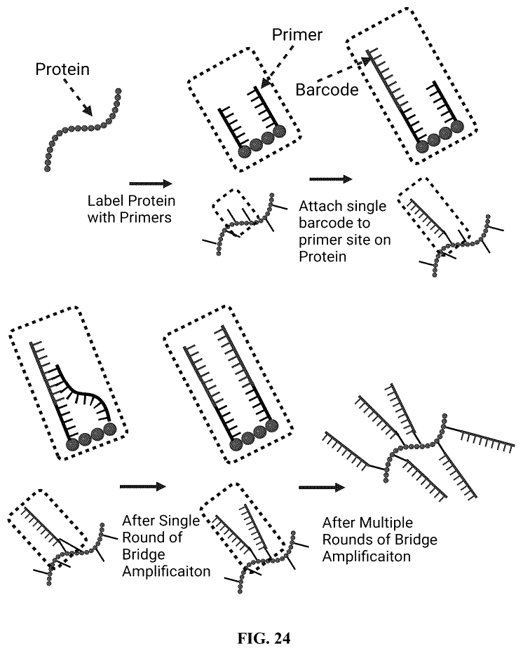

In another example, a peptide may be barcoded at multiple locations for a given peptide using bridge amplification. In such an approach, and as schematically depicted in FIG. 24, a peptide or protein may be labeled at multiple sites with a nucleic acid primer. A nucleic acid barcode molecule may be provided, which can anneal to the nucleic acid primer (not shown) or be ligated to the nucleic acid primer. Subsequent rounds of bridge amplification may be performed in order to copy the nucleic acid barcode molecule to the other primers located at other sites of the given peptide. In some examples, a peptide may be tagged with multiple copies of the nucleic acid primer, and barcode sequences may be provided sparsely, such that only one nucleic acid primer per peptide is extended by polymerase extension. Subsequent rounds of bridge amplification can result in a peptide having the same barcode sequence at each nucleic acid primer. Subsequent fragmenting of peptides may be performed, such that peptide fragments comprise on average, a single barcode. Accordingly, in some cases, the output such an amplification approach may be peptides with individual barcodes generated from fragmenting multi-labeled proteins where peptides from the same protein have the same barcodes.

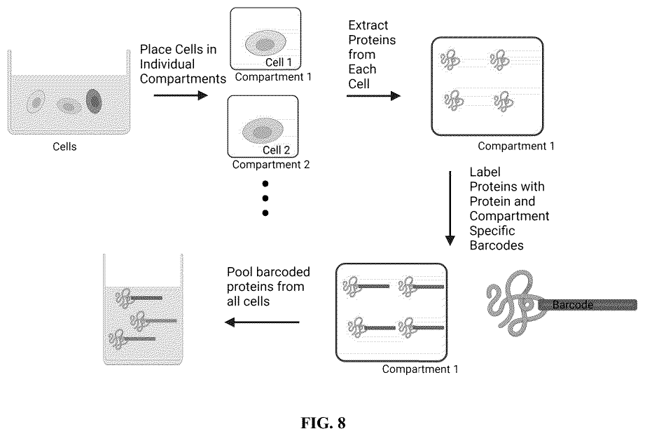

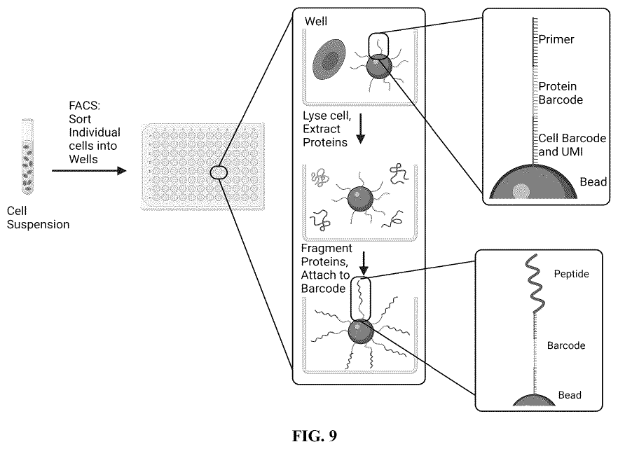

FIGS. 8-10 schematically illustrate an example workflow for processing a cell sample in partitions to obtain barcoded peptides. A sample of cells may be partitioned into individual partitions or compartments (e.g., droplets, microwells) such that at least a subset of the partitions comprise a single cell. The partitions may then be treated with a lysing agent to lyse the cells and release the proteins from the cells into the partition. The proteins may then be labeled with a partition-specific barcode (e.g., using a barcode bead, see FIGS. 8-9), such that all peptides or proteins arising from a single compartment comprises the same barcode. In some examples, the barcodes comprise nucleic acid barcode molecules, and the barcode sequence can be used in downstream processing, e.g., via sequencing, to identify the partition or cell from which a peptide originated. The nucleic acid barcode molecule may comprise any additional useful sequences, e.g., UMIs, primer sequences, etc. The nucleic acid barcode molecules within a given partition may be provided tethered to a substrate (e.g., bead).

Bulk Processing: A biological sample may be processed in bulk. For example, a biological sample may be processed to obtain a suspension of cells, which may be directly lysed in the suspension, without partitioning of cells in individual compartments. Cells may be lysed in bulk using any useful approach, e.g., as described above and optionally subjected to further processing, e.g., homogenization, protease inhibition, denaturation, protein processing (e.g., chemical treatment, fragmentation), or a combination thereof. A biological sample may be subjected to pre-processing prior to cell lysis or protein extraction. Such pre-processing may include removal of debris, purification, filtration, concentration, or sorting.

Spatial barcoding: A biological sample may comprise a tissue sample comprising multiple cells. Tissue samples may be processed using an approach to retain spatial information (e.g., to identify peptides from individual cells), e.g., using spatial barcodes. For instance, a 2-D or 3-D tissue sample may be provided, and individual cells or locations within a tissue sample may be contacted with a plurality of spatial barcodes (e.g., nucleic acid barcode molecules) comprising different barcode sequences. The different barcode sequences may be attributed to a particular location in the 2-D or 3-D tissue sample, which may correspond with a location of a cell. For example, spatial barcodes may be provided using deterministic methods such as two-photon patterning, or stochastic methods such as PCR, to assign different segments of the 2-D or 3-D tissue sample with unique spatial barcodes. Accordingly, peptides that are labeled with spatial barcodes may retain spatial or positional information of a peptide or protein or be attributed back to a single location within a tissue sample, or back to a single cell.

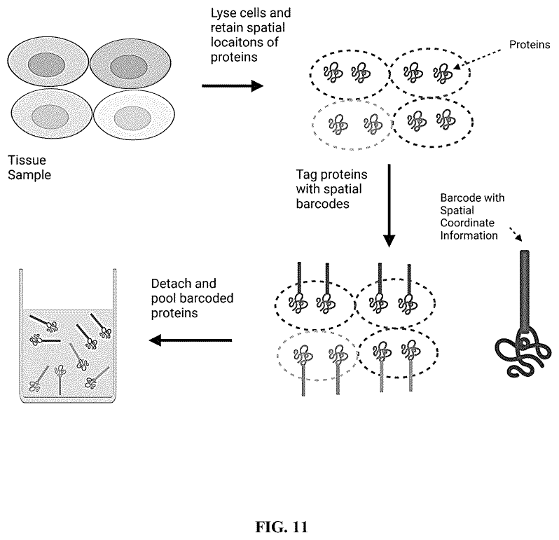

FIG. 11 schematically illustrates an example workflow of spatial barcoding of a tissue sample. A tissue sample comprising multiple cells (illustrated as a 2×2 array of cells) may be provided. The tissue sample may be subjected to lysis or fixation and permeabilization to provide access to the proteins contained within the multiple cells. Spatial barcodes, e.g., nucleic acid barcode molecules, may be provided. The spatial barcodes may comprise coordinate or location information. In an example, each cell may be contacted with a different spatial barcode, or portions of cells may be contacted with different spatial barcodes, which may optionally be pre-indexed (e.g., using imaging, or deterministic spatial barcoding approaches). Further processing of the peptides may be performed, as described elsewhere herein. As the peptides are labeled with the spatial barcodes, each peptide having a spatial barcode may be attributed back to its originating coordinate or location, which can help identify the originating cell from which a peptide arises.

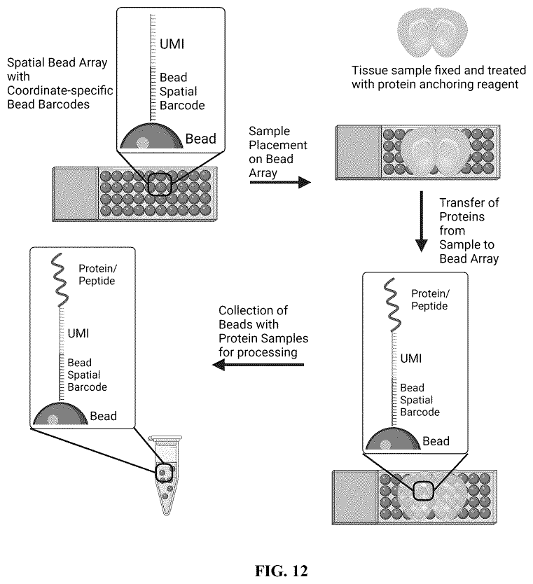

FIG. 12 schematically illustrates another example workflow of spatial barcoding of a tissue sample. A spatial barcode array may be provided on a substrate (e.g., a glass microscope slide, a hydrogel mesh). In some instances, the spatial barcodes may be directly conjugated to the substrate, or they may be provided on barcoded beads. In an example, a plurality of beads each comprising different barcode sequences may be arranged in an array on a substrate. Each bead may comprise a spatial barcode comprising a spatial barcode sequence, and optionally, a unique molecular identifier (UMI). A tissue sample (e.g., a fixed tissue sample) may be placed adjacent to (e.g., overlayed onto) the spatial barcode array. The tissue sample may then be subjected to conditions sufficient to transfer the peptides or proteins to the spatial barcode array. For example, the peptides or proteins may be transferred via passive transport, e.g., diffusion or Brownian motion, or via active transport, e.g., electrophoresis, pressure-driven flow, etc. The peptides or proteins may be attached to the spatial barcodes, e.g., using a linker (e.g., comprising amine-reactive groups, or click chemistry groups such as azide, alkyne, or other functional moieties such as aldehyde groups or NHS or carboxylic groups), conjugation chemistry, or an anchoring agent to generate barcode-tagged (barcoded) proteins. Examples of anchoring agents include fixatives, such as formaldehyde, paraformaldehyde, glutaraldehyde, or monomers for incorporation into a hydrogel, e.g., Acryloyl-X, acrylamide, N-(3-Aminopropyl)methacrylamide, or N-(3-Aminoethyl)methacrylamide, or benzophenone. Anchoring agents may also comprise multi-functional linkers, e.g., Acryloyl-X, Biotin-NHS, Biotin-PEG-Amine, DBCO-NHS, DBCO-amine. For bead arrays, the plurality of beads may be collected from the sample for further processing.

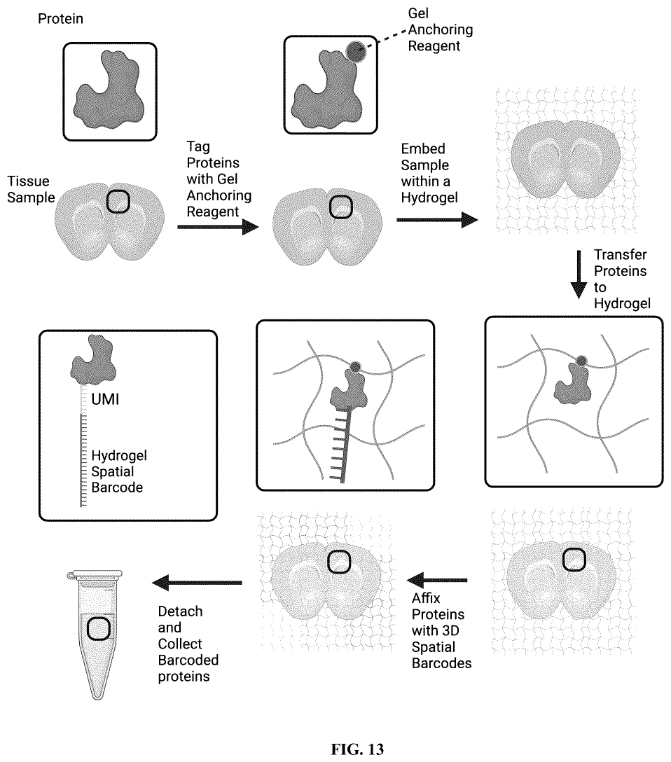

FIG. 13 schematically shows an example of tagging proteins in samples with spatial barcodes using a 3D hydrogel matrix. Intact samples can be embedded in a 3D hydrogel (e.g., polyacrylamide, polyacrylate, Expansion microscopy (ExM) gel), and proteins from samples are transferred to the hydrogel via a gel anchoring reagent (e.g., benzophenone, which can allow for photocapture). Spatial barcodes may be provided such that a pre-indexed coordinate or location comprises a unique spatial barcode. The spatial barcodes may be attached to the hydrogel matrix. The barcoded proteins may subsequently be released or detached from the hydrogel matrix for further processing. Release or detachment may be obtained using enzymatic approaches (e.g., endonuclease cleavage), chemical approaches, or mechanical approaches. The spatially barcoded proteins may then be identifiable in downstream processing operations, e.g., sequencing, to determine the barcode and the originating location of the protein.

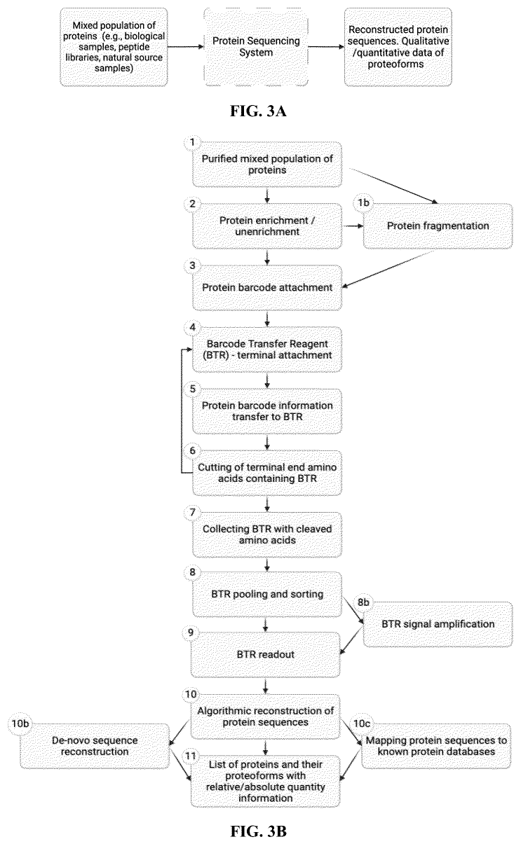

It will be appreciated that any useful combination of sample and protein processing operations may be performed prior to, during, or subsequent to the sequencing operations (e.g., ex situ peptide analysis) described herein. For example, FIG. 3A schematically depicts an example workflow for sequencing a peptide from a sample. The sample may comprise a mixed population of proteins, e.g., biological samples, peptide libraries, natural source (e.g., environmental) samples, which may be processed (e.g., peptides can be extracted, fragmented, barcoded, etc.). The processed peptides may be used as an input for peptide sequencing analysis, e.g., using barcode transfer reagents, as described elsewhere herein, to serially barcode individual terminal amino acids for identification. The serially barcoded individual terminal amino acids may be analyzed, e.g., using nucleic acid sequencing, and the barcodes may be used to reconstruct protein sequences, as well as qualitative or quantitative data on the peptides (e.g., identification or quantification of proteoforms). FIG. 3B schematically shows an example workflow for characterizing a peptide or protein. For example, a sample comprising a mixed population of proteins may be processed to obtain the proteins (e.g., via enrichment). The proteins may be fragmented. The proteins (or fragmented proteins) may be subjected to barcoding, as described herein. The barcoded proteins may then be subjected to ex situ sequencing analysis, as described herein (e.g., see FIG. 1), which may comprise attachment of a barcode transfer reagent (e.g., a nucleic acid barcode molecule) to a terminal amino acid to generate a BTR-AC, transfer of the protein barcode to the BTR-AC, and cleavage of the BTR-AC from the protein. One or more rounds or cycles of the ex-situ analysis may be performed to obtain a plurality of BTR-ACs from the peptide. The BTR-ACs may be collected and in some instances, pooled and sorted. Sorting of the BTR-ACs may be performed based on a quality of the BTR-AC, e.g., according to the amino acid type, and may be achieved using a binding agent that has specificity to a single or multiple amino acid types. In some instances, the barcodes of the BTR-AC and the protein barcode comprise nucleic acid molecules, which can be amplified for improved signal readout. The nucleic acid molecules can be readout, e.g., using DNA sequencing to obtain the cycle number and the originating protein. Algorithmic reconstruction of protein sequences may be performed using the readout of the barcodes. The proteins can be mapped back to protein sequences of known protein databases to identify the protein. Alternatively, the readout may be useful in de novo sequence reconstruction. Absolute or relative quantification of the proteins or proteoforms may be obtained.

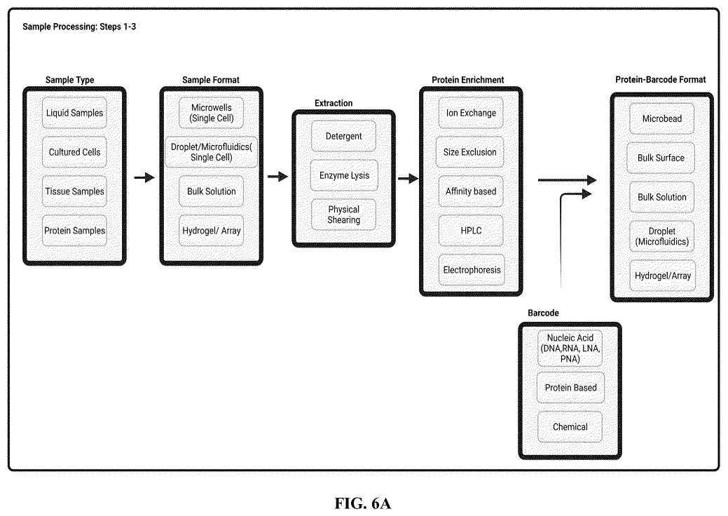

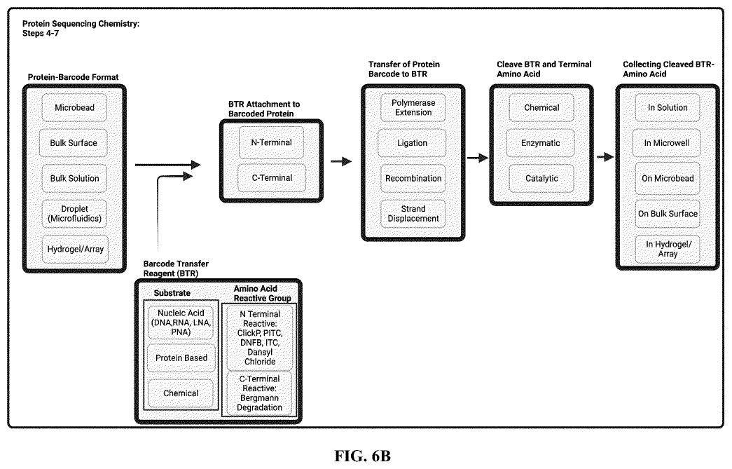

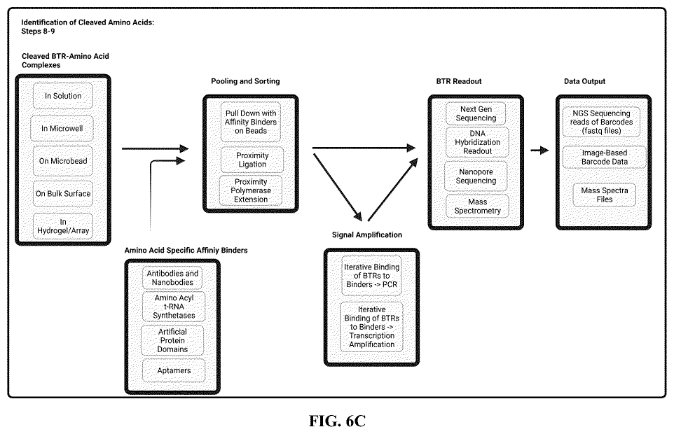

FIG. 6A schematically shows an example workflow of processing a sample, with alternatives for sample type, sample format, extraction, protein processing, and protein barcoding. A sample may comprise or be obtained from liquid samples, cultured cells, tissue samples, or protein samples. The samples may be provided in a variety of formats, or partitioned, such that the samples are provided in microwells (e.g., for single cell protein analysis), droplets or emulsions (e.g., in a microfluidic device), in bulk solution, or adjacent to a hydrogel, array, or other substrate. Extraction of the proteins (e.g., from cells) may be performed, e.g., by lysing cells in the sample using detergents (e.g., SDS, RIPA, Triton-X 100, CHAPS), enzymatic digestion or lysis, or physical shearing (e.g., sonication, electroporation). In some instances, the proteins may be further enriched or purified. Examples of protein purification or enrichment techniques include ion exchange chromatography, size exclusion chromatography, affinity-based separation, HPLC, and electrophoresis. The proteins may be barcoded (e.g., with nucleic acid barcodes, non-canonical or modified barcodes such as LNA or PNA, peptide barcodes, or chemical barcodes, e.g., fluorophores, mass tags, radioisotopes, etc.). Barcoding may occur such that the barcoded peptide is provided on a bead, a bulk surface, a bulk solution, a droplet, or a hydrogel or array.

FIG. 6B schematically shows an example workflow of sequencing a barcoded peptide, using the methods provided herein. The barcoded proteins may be provided on a bead, bulk surface, bulk solution, droplet, hydrogel, or array and may be combined or contacted with a barcode transfer reagent (BTR). The BTR may comprise a barcode (e.g., nucleic acid, peptide, or chemical) and an amino acid reactive group, as described elsewhere herein. Non-limiting examples of N-terminal reactive moieties include PITC, DNFB, isothiocyanates, dansyl chloride. Non-limiting examples of C-terminal reactive moieties include Bergmann reagents, isothiocyanates, etc. The BTR may attach or couple to a terminal amino acid (e.g., N-terminal or C-terminal amino acid) to generate a BTR-AC. The barcode of the barcoded peptide, or portion thereof, may be transferred to the BTR, e.g., using a polymerase extension reaction, ligation, recombination, or strand displacement. The terminal amino acid of the BTR-AC may be cleaved from the peptide, e.g., chemically, enzymatically, or catalytically. The liberated BTR-AC may then be collected from the solution, microwell, microbead, bulk surface, hydrogel, array, etc. for further analysis.

FIG. 6C schematically shows example workflows of further analysis that can be performed on a plurality of BTR-ACs to sequence a peptide. A plurality of liberated BTR-ACs may be combined with or contacted with amino acid-specific binding agents (e.g., affinity binding agents such as antibodies or nanobodies, single-chain variable fragments, amino acyl tRNA synthetases, artificial protein domains, aptamers). The amino acid-specific binding agents may be used to sort the BTR-ACs. For example, the BTR-ACs comprising one type of amino acid may be sorted to a first group; BTR-ACs comprising a different type of amino acid may be sorted to a second group, etc. Such sorting may be achieved, for example, using a pull-down assay (e.g., if the binding agents are coupled to magnetic or fluorescent beads that can be sorted). In some instances, the amino acid-specific binding agents may comprise binding agent barcodes, which may be directly coupled to the binding agents or to a substrate that is coupled to the binding agent. The binding agent barcodes may identify the binding agent or the binding partner (e.g., specific amino acid). In some instances, the binding agent barcodes are transferred or copied to the BTR-ACs, e.g., using proximity ligation, proximity polymerase extension, etc. (e.g., as shown in FIGS. 2, and 21-23). Optionally, signal amplification, e.g., amplification of the BTR-AC barcodes, iterative binding of the BTR-ACs to the binding agents, may be performed. The BTR-AC barcodes (or amplified product thereof) may then be subjected to detection, such as barcode readout using DNA sequencing (e.g., NGS sequencing, DNA hybridization assays, nanopore sequencing) or mass spectrometry. The output data from such detection can include NGS sequencing data (e.g., sequencing reads of barcodes), image-based barcode data (e.g., from DNA hybridization assays), or mass spectra. The output data may be computationally assessed to determine the identity of the amino acid, the order or sequence (e.g., cycle number) in which the amino acid is present in a peptide, and the originating peptide.

In additional aspects of the present disclosure, provided herein are systems, compositions and kits for performing single molecule protein sequencing. The systems, compositions, and kits may comprise a barcode transfer reagent, which may comprise a primer sequence that is configured to bind or couple to a barcode molecule (e.g., a peptide comprising a nucleic acid barcode molecule), and a chemical moiety that can react with an amino acid (e.g., NTAA, CTAA, internal amino acid, or combination thereof). The systems, compositions, and kits may comprise additional useful items, such as reagents, catalysts, ions, buffers, enzymes, labeling agents, and instructions for use.

In another aspect of the present disclosure, provided herein is a method for processing a sample comprising a peptide, comprising fixing the sample, permeabilizing and digesting the sample, transferring the peptide to an array, tagging the peptide with a plurality of barcodes to generate a tagged peptide, imaging the peptide or extending a barcode of the plurality of barcodes to a neighboring or adjacent barcode, releasing the tagged peptide from the array, and collecting the tagged peptide for further processing. The further processing may comprise peptide or protein sequencing, as described herein.

Another aspect of the present disclosure relates to processing proteins or peptides for better detection. An example method may comprise converting an amino acid or post-translational modification on a peptide to a chemical group, tagging the peptide with a barcode, contacting a BTR to a terminal amino acid or derivative thereof of the peptide to generate a BTR-AC, cleaving the BTR-AC from the peptide to release the BTR-AC, and detecting the chemical group, e.g., using a binding agent, thereby detecting the amino acid of post-translational modification.

In yet another aspect of the present disclosure, provided herein is a method for barcoding a peptide. The method may comprise tagging the peptide with a plurality of barcodes comprising different barcode sequences, coupling a dual primer linker sequence to two adjacent barcode sequences of the plurality of barcodes, copying or transferring one of the adjacent barcode sequences of the two adjacent barcode sequences to the other adjacent barcode sequence of the two adjacent barcode sequences, and sequencing the peptide. The copying or transferring of the barcode sequences may occur via a nucleic acid extension reaction (e.g., using a polymerase). The sequencing may be performed using a BTR, as described elsewhere herein.

In another aspect, disclosed herein is a method for sequencing native proteins. The method may comprise tagging or barcoding a native folded protein with a plurality of barcodes to generate a tagged or barcoded protein, fragmenting the tagged or barcoded protein into a plurality of peptides, contacting a BTR to a peptide of the plurality of peptides to generate a BTR-AC, cleaving the BTR-AC, contacting the BTR-AC with a binding agent, and reading out the barcode information from the BTR-AC. One or more operations may be repeated, e.g., to generate a plurality of BTR-ACs to serially sequence the peptide, to detect barcodes from all the peptides fragmented from the protein, etc. Such a method may be useful in identifying amino acids that are exposed, on the surface, or solvent-facing.

Another aspect of the present disclosure relates to identifying and sequencing proteins based on a functional aspect of a protein. Such an example method may comprise performing a functional assay of a peptide or a library of peptides to identify peptides of interest, separating the peptides of interest to generate substantially isolated peptides, tagging the substantially isolated peptides with peptide-specific barcodes, and sequencing the tagged peptides, e.g., as described elsewhere herein.

Another aspect of the present disclosure relates to screening a library of proteins which may arise from a single encoding DNA or RNA sequence. In an example, a method may comprise generating a library of proteins from a single encoding DNA or RNA sequence, e.g., by introducing substitutions during translation or other mutagenesis technique, tagging one or more proteins from the library of proteins with a barcode, and sequencing the tagged proteins. The sequencing may be performed using the methods described herein, e.g., contacting the tagged proteins with a BTR comprising barcode information to generate a BTR-AC, cleaving the BTR-AC, optionally repeating the process to generate a plurality of BTR-ACs, contacting the BTR-AC or plurality of BTR-ACs with one or more binding agents, and reading out barcode information from the BTR-ACs.

In yet another aspect of the present disclosure, provided herein is a method for generating a molecular target profile or to determine the location of binding of a molecule on a peptide. The method may comprise mixing a molecule with a first protein target to form a complex and exposing the complex to a protease to generate one or more fragments of the complex, exposing a second protein target to a protease to generate one or more fragments of a protein target, labeling or barcoding the one or more fragments of the protein target and the one or more fragments of the complex to generate one or more barcoded fragments, and sequencing the one or more barcoded fragments. Sequencing may be performed using BTRs, as described herein, and may be useful in evaluation of one or more features of the one or more fragments of the complex and one or more features of the one or more fragments of the protein target.

Also provided herein are methods for preparing multimeric binding agents, comprising linking or fusing one or more binding agents.

In another aspect, provided herein is a method of conjugating a chemical tag to a peptide or protein, comprising tagging a peptide or protein with a chemical tag that is attached to an enzyme substrate, and using the chemical tag to at conjugate the peptide or protein to a surface or substrate.

### Method Overview and Example Procedures

Sample Preparation. The present disclosure provides for approaches for preparing samples for peptide sequencing, including peptide extraction, purification, and peptide barcoding. This process may begin with peptides labeled with unique identifier barcodes (UMI peptides/proteins). These barcoded peptides can be prepared from protein samples, where proteins are chemically or enzymatically digested into peptides, and the resultant peptides are conjugated with peptide specific barcodes. These molecular barcodes may be appended to peptides chemically (to either N or C termini, or to internal sites) or enzymatically. Each peptide may comprise a single peptide barcodes or multiple peptide barcodes that are the same or different. A variety of molecular types can be used for barcodes: DNA, RNA, proteins, synthetic polymers or other materials.

For example, as shown in FIG. 1, DNA barcodes are used for illustration. In the case of DNA or RNA barcodes, they may contain a primer site for downstream steps as well as unique barcode sequences that identify each peptide (FIG. 1). Alternatively, proteins can first be chemically or enzymatically modified with one or multiple barcodes, and then digested chemically or enzymatically to yield peptides with unique barcodes. Furthermore, barcode tagged peptides can be prepared in solution, or on a solid support.

Design of Barcode Transfer Reagent: The reagent which reacts to the termini of peptides for the sequential removal of amino acids may have three components. The BTRs may comprise (1) a barcode containing cycle information. In principle, a variety of molecules can be used as barcodes, such as DNA, RNA, proteins, synthetic molecules, or other materials. The second possible component is (2) a primer that hybridizes to a site on the peptide barcode (this is specific to DNA/RNA). The last component is a (3) chemical moiety that becomes conjugated to either the N-, C-, or both terminal ends of a peptide.

Tagging of Peptides with Barcode Transfer Reagent, barcode information transfer, Cleavage, and Iteration): Upon addition of the Barcode Transfer Reagent to the barcoded peptides, the reagent can conjugate covalently to either the N- or C-termini of the peptide, depending on its design. As it is conjugated to a peptide, the primer region of the barcode on the reagent will attach to its target region on the peptide barcode. As an example, in the case of DNA or RNA barcodes, the primer will hybridize to its complementary region on the peptide barcode. In the case of DNA or RNA barcodes, a polymerase is then added which will extend the primer region of the reagent copying the barcode information of the peptide in the process. While DNA barcodes are used here as an example, similar procedures can be implemented for other types of molecular barcodes. At this stage, the Barcoded Reagent contains the cycle information as well as the peptide barcode information; in other words, the identity of the peptide and the location of the terminal amino acid.

The Barcode Transfer Reagent and the terminal amino acid is then chemically cleaved off the peptide that it is attached to, liberating the barcode-amino acid complex (BTR-AC) from the peptide in the process. Alternatively, this cleavage can happen enzymatically. The removal of the terminal amino acid along with the reagent resets the peptide for another round or cycle of reaction with a new reagent starting from the next amino acid. This process continues with amino acids being liberated from the termini one at a time, tagged with barcodes in the process, for a desired number of steps or until all the available amino acids on a peptide are released. Importantly, with each cycle, the released barcode-amino acid complexes contain information for the cycle number (not necessary but would be advantageous) as well as the identity of the peptides of origin for the released amino acids. The present disclosure further provides embodiments where barcode transfer comprises transferring information from the sequencing reagent to the peptide barcode.

FIG. 5 illustrates a scheme for transfer of barcode information from the BTR reagent to the protein barcode using ligation and polymerase extension steps. Once a BTR, containing its own unique barcode, reacts to a terminal end, a ligase is used to attach the free end of the BTR to the free end of the protein barcode. Then, a polymerase extension step creates a double stranded segment between the two ligated regions. Finally, cleavage of the double stranded segment with a restriction enzyme releases the protein barcode with a copy of the BTR barcode sequence. The BTR along with the terminal amino acid is cleaved and the whole process repeated from the new terminal amino acid.

Identifying Released Amino Acids, their Origin Peptides, and Sequential Order via Ex-situ Molecular Analysis: The steps of iteratively conjugating terminal amino acids on peptides with the Barcode Transfer Reagent, copying over the peptide barcode, and subsequent cleavage results in a set of individual liberated amino acids tagged with barcodes containing peptide barcode information as well as cycle information. At this stage, ex-situ analysis is performed to first, identify and segregate amino acids by type, and second, read out the barcode information accompanying each liberated amino acid.

While a variety of methods can be used to identify amino acids, such as using binding agents, mass spectrometry, or nanopore readers among others, the use of binding agents is discussed here as an example. Barcode-amino acid complexes can be pulled down and segregated by identity using binding agents specific to amino acids, such as antibodies, nanobodies, modified amino acid tRNA synthetases, Edmanase, somamers, proteins or other similar reagents. This pull-down step can occur in different formats: e.g., with binding agents on beads, or on a solid surface. At this stage, these amino acids can also be segregated based on post-translational modifications, such as phosphorylation or nitrosylation marks, using the appropriate binding agents.

Once separated by identity, various approaches can be used to read out the barcode information depending on the type of barcode used. In the case of DNA or RNA barcodes, Next Generation DNA Sequencing (NGS) (or other DNA sequencing approaches) is used to read out the sequence that accompanies each amino acid. This step is preceded by an amplification step using NGS library preparation techniques to generate sufficient samples for sequencing. The sequencing can be carried out using sequencing by synthesis approaches (e.g., Illumina Sequencers, PacBio sequencers), sequencing by ligation (e.g., SolID), or nanopore-based sequencing (e.g., Oxford Nanopore). In addition, DNA or RNA barcodes may also be identified using sequence hybridization or ligation-based approaches. For other types of barcodes, such as protein or synthetic polymers, affinity tags or other types of chemical labels may be used.

The barcode tagged to each amino acid contains information relating to the barcode of the peptide from which the amino acid was cleaved as well as the cycle information. Using this information, the order of amino acids on their parent peptides can be determined computationally. Potential errors arising from incomplete reactions or other sources can be addressed using statistical and machine learning techniques that connect peptide information to proteomic data.

Improving Signal-to-Noise Ratio via repeated interrogation, amplification methods and Barcode information Transfer: The accuracy of the ex-situ analysis can be enhanced by repeatedly interrogating the same BTR-ACs. As an example, when using amino acid specific binding agents to pull down the BTR-ACs, once the BTR-AC is bound to the binding agent, the barcode information from the BTR-ACs can be copied. The copied information can be used for readout of the barcode while the BTR-AC is dissociated from the binding agent and pulled down again. These steps, pulling down complexes, copying barcode information, and dissociating, can be repeated as many times as needed to achieve a satisfactory signal to noise ratio. In the case of DNA or RNA barcodes, the copied barcode information can be amplified for readout using sequencing similar to DNA deep sequencing approaches.

In addition to copying barcode information, proximity ligation can be used to connect the identity of the binding agent to the identity of the barcode. As one example, when using binding agents on beads or on a surface, the binding agents can comprise a barcode that uniquely identifies them. When the barcode-amino acid complex is pulled down by the binding agent, the peptide/amino acid specific barcode can be ligated to the barcode identifying the binding agent. In the case of DNA or RNA barcodes, these two barcodes can be transferred/copied chemically or enzymatically, and the information can be readout using DNA sequencing or other methods common for identifying nucleic acids. FIG. 2 provides an example, as described elsewhere herein.

An example sample processing workflow and peptide sequencing analysis approach is provided below.

Step 1. Extraction of Proteins from Samples

### Context A. Single Cell Samples

The methods and systems disclosed herein may comprise preparing single cell suspensions. Methods provided herein may further comprise: (a) sorting or isolating of single cells from single-cell suspensions into individual compartments; (b) lysing the single cells; and (c) releasing and denaturing proteins from the single cells. Methods may or may not comprise subjecting the single cells to nuclease digestion to remove nucleic acids, such as RNA or DNA, from the single cells.

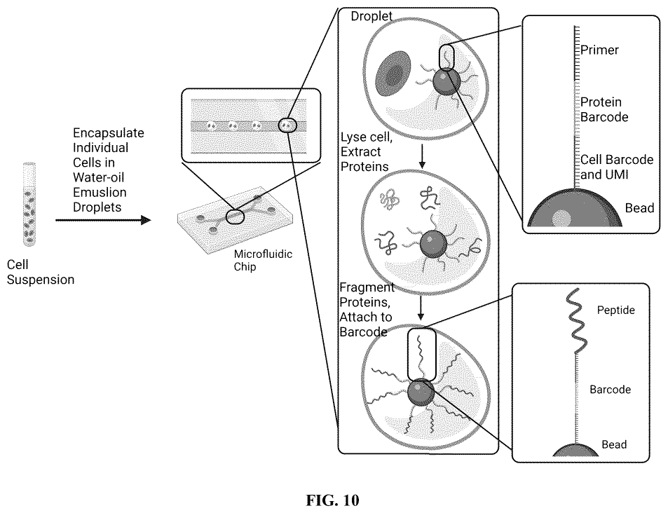

Referring again to FIG. 8, processing single cell samples may begin with cell suspensions and result in proteins with cell-specific barcodes. FIG. 9 provides a single cell-specific sample preparation for protein sequencing, which involves sorting individual cells into wells and using barcoded beads to tag and tether proteins. FIG. 10 provides single cell-specific sample preparation for protein sequencing, which involves encapsulating single cells in water-oil emulsion droplets along with barcoded beads that tag and tether proteins.

Step A1 (Input): Single Cell Suspension Preparation Example. As a first step to single cell sample preparation, single cell suspensions are prepared from biological samples by dissociating cells and placing them in a media. The sample also can be treated to de-aggregate the proteins in the sample. The sample can be de-aggregated through chemical or mechanical methods. Chemical de-aggregate methods can include but are not limited to: sodium dodecyl (SDS), Triton-X 100, 3-((3-cholamidopropyl)dimethylamminio)-1-proppanesulfonate (CHAPS), ethylene carbonate, or formamide. Mechanical de-aggregation methods can include but are not limited to: sonication or high temperature treatment. (a) Liquid Samples. In some embodiments, the single cell samples comprise liquid samples. For example, one set of samples involve cells that are already dissociated, such as bacterial liquid cultures, mammalian liquid cultures, and blood serum samples. In these cases, the samples are centrifuged to isolate cells, which are then resuspended in a suitable media, such as Dulbecco's Phosphate Buffered Saline (DPBS). (b) Cultured Cells. Samples can also include cultured cells, where cells are prepared adhered to a solid surface, such as petri dishes. Cultured cells can include mammalian cell samples used in medical research, patient-derived cell samples, or induced pluripotent stem-cells among others. Cultured cells samples are treated with a light protease, such as trypsin, to detach them from the surface, and are then collected and suspended in a suitable media. (c) Tissue or Biopsy Samples. Another possible set of samples include tissue samples. Single cell suspensions are prepared from fresh, acute tissue samples mechanically, enzymatically, or through a combination of both. Sonication, the application of ultrasonic waves to a sample, can be used to break down a tissue sample into its constituent individual cells. In addition, enzymes such as pronase that break down the extracellular matrix of tissues that hold cells together can be applied to tissues to generate single cell suspensions. The dissociated cells can then be stored in a suitable buffer, such as DPBS.

Step A2: Sorting. Once cell suspensions are prepared, individual cells are sorted or isolated into individual compartments or partitions for further processing. Cell specific barcodes (e.g., beads with unique DNA barcodes) will be present in each compartment to tag the protein contents of each cell with a unique identifier (See Step 3). Sorting of cell suspensions may comprise, for example, one or more of (a) FACS sorting into well-plates; (b) microfluidic sorting into droplets; (c) gravity sorting into microwells; and/or (d) mechanical cell picking into wells. (a) FACS Sorting into Well-Plates. In some embodiments, cell suspensions are sorted into individual wells in plates using fluorescence activated cell sorting (FACS). For example, endogenous fluorescence of cells or applied fluorescence label may be used to sort cells into individual well plates. (e.g., Sort-Seq). (b) Microfluidic Sorting into Droplets. In other embodiments, microfluidic sorting into liquid or solid droplets may be used to sort cell suspensions. Further, microfluidic approaches common in transcriptomics, such as Drop-Seq, can be used to sort cells into individual water-oil emulsion droplets. (c) Gravity Sorting into MicroWells (e.g., SeqWell). In other embodiments, sorting may be performed via gravity sorting into microwells, such as SeqWell. Further, cells may be sorted into microwell plates with each well designed to hold a single cell. In this approach, cells in a suspension are directly applied to the plates and allowed to settle into individual wells. (d) Mechanical Cell Picking into Wells. An alternative sorting method may comprise mechanically isolating cells and placing cells into wells in a well-plate. In the case of cultured cells, micropipettes can be used to aspirate individual cells and place them in a desired well on a plate. This process may be repeated for as many cells as needed.

Step A3: Lysis—Permeabilization and Extraction. Once sorted into individual compartments, individual cells are lysed to release their protein content. While various lysis methods exist, protease inhibitors are generally added to inhibit endogenous proteases that might degrade protein content. (a) Detergent Treatment. In some embodiments, lysis is performed via detergent treatment. In such an embodiment, isolated single cells can be lysed by exposing them to detergents that solubilize lipid membranes, thereby liberating cellular contents. Detergents for cell lysis may include, Triton-X 100, sodium dodecyl sulfate (SDS), or sodium deoxycholate among others. Often, detergent treatment of cells is accompanied by high temperature incubation to facilitate lysis. (b) Detergent Treatment and Enzyme Digestion. In addition to detergents, lysis buffers can include enzymes that digest extracellular matrix molecules to facilitate lysis. Such enzymes include collagenase and protease among others. (c) Sonication. Sonication can also be used in combination with detergent to lyse cells. Cells are placed in a detergent solution, and sonication is applied to the sample while incubating at a high temperature (e.g., 60° C.). (d) Freeze-thaw. Freeze-thaw methods may also be used. In such embodiments, after isolation, cells can be lysed through repeated cycles of freezing and thawing. Iterative freeze-thaws disrupt the membrane integrity of cells resulting in the release of their cellular contents. Commonly, any practical combination of the above methods can be applied depending on the sample type.

Step A4: Nuclease Digestion (Optional). In addition to proteins, nucleic acids are one of the major macromolecular components of cells. Nucleic acids, such as RNA and DNA, can be removed from samples to remove potential interference during protein sequencing. After lysis, DNA can be removed by adding endonucleases such as DNAse I, and RNA can be removed using a cocktail of RNAse enzymes.

Step A5: Denaturation and Reduction. Following the lysis of cells, release proteins are denatured and reduced. Denaturation renders different parts of proteins accessible for further processing, while reduction breaks disulfide bonds to yield reduced cysteine residues. (a) Heat Denaturation and Reduction. In some embodiments, heat denaturation and reduction are used, such that released proteins from cells can be denatured and reduced via treatment agents (e.g., SDS, Urea, Guanidine) along with reducing agents (e.g., dithiothreitol, beta mercaptoethanol, TCEP) at a high temperature. (b) Enzyme Denaturation. In other embodiments, enzyme denaturation is used. For example, enzyme degradation may involve using proteins such as ClpX which are able to bind and denature proteins. Output (a). In some embodiments, denaturation, and reduction results in denatured proteins from individual cells in microwells. Output (b). In some embodiments, denaturation and reduction may result in denatured proteins from individual cells encapsulated in droplets (with a cell per droplet) along with barcoded beads in a microfluidic system.

### Context B. Bulk Cell Suspensions or Cultured Cells

Bulk cell suspensions or cultured cells may be extracted to generate denatured proteins from many cells in a solution. Various methods may be employed.

Step B1 (Input): Suspending cells in media. Cells may be suspended in liquid samples or as cultured cells. For example, liquid samples may include centrifuge samples (e.g., Liquid culture, serum), and exchange into protein extraction media. Cultured cells may be suspended via trypsinization of cultured cells and suspension in protein extraction media.

Step B2: Homogenization, Permeabilization and Extraction. Homogenization, permeabilization and extraction may be performed via a variety of methods. For example, detergent treatment may be employed by adding, for example, Triton-x 100, or SDS (a protease inhibitor may be added here). Detergent treatment and enzyme digestion may also be employed using, for example, Triton-x 100 and trypsin, lysozyme or papain. Further, freeze thaw methods or sonication—discussed above—may be employed to achieve homogenization, permeabilization and extraction. Finally, ultracentrifugation and sucrose gradients may be employed.

Step B3: Nuclease Digestion. Next, nuclease digestion is performed using, for example, endonuclease digestion of DNA and RNA.

Step B4: Denaturation and Reduction. Denaturation and reduction may be achieved via detergent and reducing agents (e.g., SDS, DTT, Beta-mercaptoethanol, urea, or TCEP). Alternatively, heat denaturation and reduction or enzyme denaturation may be employed.

### Context C. Bulk Tissue Samples

Step C1: Homogenization, Permeabilization and Extraction. In cases where biological tissue samples are used (e.g., tissue biopsy, animal tissue, plant tissue), homogenization, permeabilization and extraction (i.e., Step C1) may be performed via detergent treatment and enzyme digestion. For example, Triton X-100, SDS, or Pronase may be used. In some embodiments, homogenization, permeabilization and extraction may be performed via sonication and detergent treatment (e.g., Triton X-100, SDS).

Step C2: Nuclease Digestion of Endogenous DNA and RNA. Endonuclease digestion of DNA and RNA may be performed via, for example, endonuclease digestion of DNA and RNA (e.g., DNAse I; RNAse Cocktail).

Step C3: Denaturation and Reduction. Denaturation and reduction may be performed, for example, using detergent and reducing agent (e.g., SDS, DTT, Beta-mercaptoethanol, urea, TCEP). Heat denaturation and reduction or enzyme denaturation may also be employed to obtain denatured proteins from a tissue sample in solution.

Context D. Preserving Spatial Information from a Sample in a Hydrogel Using In Situ Analysis

In this approach, the 3D location of proteins within a biological sample is preserved by retaining proteins from biological samples within a dense hydrogel mesh. The hydrogel mesh preserves the relative 3D position of proteins. In such an example, the input comprises intact specimens such as cultured cell samples or tissue samples (e.g., tissue biopsy). FIG. 11 provides a generalized sample processing workflow for generating proteins bearing spatial barcodes from intact specimens such as tissue samples. FIG. 12 illustrates preservation of spatial location of proteins using a spatially organized 2D bead array. Intact samples are placed on the bead array and proteins are transferred from the sample to the beads via a protein anchoring reagent, which attaches proteins to spatial barcodes on beads. These protein bearing barcoded beads are then collected for downstream processing. FIG. 13 depicts an example of tagging proteins in samples with spatial barcodes using a 3D hydrogel. Intact samples are embedded in a 3D hydrogel, and proteins from samples are transferred to the hydrogel via a gel anchoring reagent. Once transferred, deterministic methods such as two-photon patterning or stochastic methods such as PCR, are used to assign different segments of the 3D hydrogel with unique spatial barcodes. Finally, proteins now bearing barcodes, which denote their original 3D locations, are detached from the gel and collected for downstream processing.

Step D1: Fixation of sample and functionalization of protein samples with hydrogel anchoring reagent. (a) Trifunctional Anchoring Reagent. One option for fixation of the sample and functionalization of the protein samples is to fix tissues with a fixative, such as formaldehyde, and tag proteins with chemical moieties for incorporation into hydrogel, such as Acryloyl-X, Acrylamide, or N-(3-Aminopropyl)methacrylamide, or N-(3-Aminoethyl)methacrylamide, for example. (b) Reactive Unique Molecular Identifier (UMI) barcodes. Alternatively, proteins may be tagged with a library of DNA barcodes that can be incorporated into a gel to generate reactive UMI barcodes. Such reactive barcodes have functionalities for reacting to proteins such as amine, azido, alkyne, aldehyde, N-hydroxy succinimide (NHS), or carboxylic groups.

Step D2: Embedding samples in a hydrogel. Here, the sample is embedded in a hydrogel (e.g., polyacrylamide, polyacrylate, ExM) that permeates the entire sample. Proteins functionalized with anchoring reagent or reactive barcodes will be attached to the hydrogel.

Step D3: Homogenization and Permeabilization. One option for step D3 (homogenization and permeabilization) is to separate proteins from one another by using heat denaturation, such as through a combination of heat and detergents, such as Sodium Dodecyl Sulfate (SDS). Alternatively, enzyme denaturation can be used, so that proteins are dissociated using a light protease digestion (e.g., proteinase K, GluC).

Step D4: Assigning 3D Spatial barcodes to proteins. By Step D4, proteins from the sample have been transferred to the hydrogel. In Step D4, these proteins will be tagged with barcodes (e.g., DNA, RNA) whose sequences denote their relative position in 3D within the hydrogel. Tagging may be performed via a variety of methods. For example, one option is using two-photon printing of barcodes on proteins. In this method, two-photon patterning may be employed to label proteins with 3D tags. Another example may utilize stochastic assignment of spatial barcodes to proteins. In particular, PCR may be used to randomly amplify in situ spatial barcodes within the hydrogel which will then associate with proteins. The sequence may be used to identify the spatial location of barcodes (Step D6 below).

Step D5: Release of proteins with unique identifier barcodes as well as 3D spatial barcodes. Once proteins have been tagged with UMIs and spatial barcodes, they may be released from the hydrogel for further processing. Alternatively, processing may occur in the hydrogel.

Step D6: Mapping of Spatial barcodes to Spatial Coordinates. Once proteins with spatial barcodes (patterned or randomly amplified in situ) have been released from the hydrogel, the physical location of these barcodes in 3D coordinates needs to be established. This is accomplished by carrying out in situ sequencing of the barcodes remaining in the hydrogel. For example, one option is to process the hydrogel through in-situ sequencing by synthesis to associate barcodes to 3D locations via imaging. Briefly, round or cycles of in situ sequencing with imaging on a confocal microscope are carried out. These round or cycles of in situ sequencing will produce images indicating the location of various spatial barcodes. These 3D locations will later be used to assign spatial positions to proteins.

The output of Step 1a in Context D is proteins/peptides tagged with barcodes with unique molecular identifier (UMI) for each barcode, as well as barcode sequences denoting 3D spatial location.

Context E: Preserving Spatial Information from a Sample on an Array

Here, the 2D spatial location of proteins within sample is preserved by transferring proteins from biological tissues to a 2D array of barcodes.

Step E1: Preparing 2D Spatial Array. In Step E1, a 2D array of DNA barcodes is prepared with each barcode sequence denoting a 2D coordinate location. In some embodiments, a bead array may be used. In such an embodiment, each bead contains DNA sequences denoting its 2D location in the array. In other embodiments, a printed DNA array may be used. In such an embodiment, a 2D array of DNA barcodes is printed on a surface, such as a glass slide, with each barcode noting its 2D location.

Step E2: Fixation of sample and functionalization of protein samples with anchoring reagent. In this context, the input comprises Cultured Cell samples or tissue samples (e.g., tissue biopsy). In Step E2, proteins within a biological sample are first fixed (e.g., using formaldehyde) and are functionalized with a reagent to enable them to be transferred to the 2D array. In some embodiments of the present disclosure, proteins are labeled with an anchoring reagent to transfer to an array. In such embodiments, proteins are first fixed, and then are labeled with a multi-functional anchoring reagent (e.g., Acryloyl-X, Biotin-NHS, Biotin-PEG-Amine, DBCO-NHS, DBCO-amine). Alternative methods of labeling proteins may be used as well. Such an option may include labelling proteins with unique identifier barcodes. Alternatively, after fixation, proteins can be labeled with UMI barcodes that can be transferred to the 2D array. In the case of DNA or RNA based reactive barcodes, protein reactive groups present on the barcodes may include amine, aldehyde, carboxylic, azido, alkyne, DBCO, or N-hydroxysuccinimide groups.

Step E3: Placement of Sample on an array with patterned DNA barcodes denoting spatial location. In this step, the sample to be processed is placed on top of the 2D array.

Step E4: Permeabilization and protease digestion. Various options exist for releasing proteins from the sample. Heat denaturation, for example, or enzyme denaturation may be employed. In heat denaturation, proteins are released from the sample using a combination of heat and detergents, such as Sodium Dodecyl Sulfate (SDS). For enzyme digestion, a light protease digestion is used to dissociate proteins, including, for example, using Proteinase K, GluC.

Step E5: Attachment of proteins peptides to barcodes on spatial array. In Step E5, proteins released from the sample will diffuse and attach to nearby barcodes on the 2D array via the anchoring reagent.

Step E6: Release of proteins with unique identifier barcodes as well as spatial barcodes. Once proteins have reacted to their respective barcodes on the 2D array, proteins labeled with their respective spatial barcodes can be released from the array and collected for further processing, or be processed on 2D array directly. In some cases, the labeled proteins are released via endonuclease cleavage of the protein barcode conjugate. In some cases, labeled proteins are released via chemical release of protein barcode conjugate. In some cases, labeled proteins are released via an enzymatic release (e.g., endonuclease cleavage). After this release, the output may comprise proteins tagged with barcodes, wherein each barcode comprises a UMI, and barcode sequences denoting 2D-spatial location. Alternatively, the output may comprise proteins attached to beads via barcodes with UMI for each protein, as well as spatial barcode associated with each bead.

### Context F: Environmental Protein and Peptide Samples.

In some embodiments of the present invention, samples used comprise environmental samples comprising candidate proteins (e.g., sewage samples, swabs). In such embodiments, material debris are first removed, and then the sample is concentrated and moved into an appropriate buffer for downstream processing.

Step 1b. Protein Fragmentation

In certain applications, such as protein identification, whole proteins are not necessary. Thus fragmenting proteins into peptides can be an option for more efficient processing for identification purposes. At this optional step, intact proteins extracted from samples are fragmented into short peptides.

### Context A: Protein Samples in Bulk Solution

In some embodiments, denatured and reduced protein samples are derived from bulk cell samples (B) or bulk tissue samples (A). Various mechanisms can be used for protein fragmentation, resulting in fragmentation of the protein samples into peptides in bulk solution. For example, protease digestion may be employed. Proteases that may be used include, but are not limited to, trypsin, Lys-C, and glu-C. Chemical treatment may also be used to degrade protein samples so that they become fragmented into peptides in bulk solution. For example, cyanogen bromide may be used. Other methods, such as sonication, may be used as well. In some embodiments, a combination of methods is used.

### Context B: Protein Samples in Wells or Microwells (e.g., Single-Cell Samples)

In some embodiments, protein samples in wells or microwells (e.g., single-cell samples) serve as the protein samples that will be used. Denatured and reduced protein samples from single-cell samples may be fragmented into peptides in wells or microwells via a variety of methods. For example, protease digestion may be employed. Proteases that may be used include, but are not limited to, trypsin, Lys-C, and Glu-C. Chemical treatment may also be used to degrade protein samples so that they become fragmented into peptides in bulk solution. For example, cyanogen bromide may be used. Other methods, such as sonication, may be used, alternatively or in addition. In some embodiments, a combination of methods is used.

### Context C: Protein Samples in Droplets in a Microfluidic System (e.g., Single-Cell Samples)

In some embodiments, protein samples in the form of droplets in a microfluidic system are used (e.g., single-cell samples). Denatured and reduced protein samples from individual cells in droplets in a microfluidic system, with each droplet containing protein samples form a single cell as well as a barcoded bead, may be fragmented in peptides in droplets via various means. For example, protease digestion may be employed. Proteases that may be used include, but are not limited to, trypsin, Lys-C, and Glu-C. Chemical treatment may also be used to degrade protein samples so that they become fragmented into peptides in bulk solution. For example, cyanogen bromide may be used. Other methods, such as sonication, may be used as well. In some embodiments, a combination of methods is used.

### Step 2: Protein Enrichment

At this step, proteins extracted from samples (Step 1) or proteins digested into peptides (Step 1b) are purified and separated from any extraneous cellular material/debris or unnecessary reagents.

### Context A. Denatured Protein Peptide Samples in Bulk Solution

In some embodiments, denatured and reduced protein samples are derived from bulk cell samples (Step 1Context B), bulk tissue samples (Step 1 Context C), or fragmented peptide samples in solution form bulk cell samples or tissue samples (Step 1b, A). Protein enrichment may be performed to convert the denatured and reduced protein samples to purified protein or peptide samples in a solution suitable for barcode attachment. Various methods may be used to perform protein enrichment, including but not limited to affinity columns (ion exchange), size exclusion columns, affinity precipitation (e.g., immunoprecipitation of protein classes of interest); High Performance Liquid Chromatography (HPLC), and electrophoresis.

### Context B. Denatured Protein Peptide Samples in Wells or Microwells

In some embodiments, denatured and reduced protein samples are derived from microwells (Step 1, Context A) or fragmented peptide samples in microwells from single cell samples (Step 1b, A). These samples may be subject to protein enrichment to generate purified protein or peptide samples from individual cells in microwells in a solution suitable for barcode attachment (step 3). Alternatively, these samples may be subject to protein enrichment using barcoded microbeads to generate purified protein or peptide samples from individual cells in microwells in a solution suitable for barcode attachment, wherein the barcoded microbeads are specific for each cell in each well. In some embodiments, protein enrichment may be performed by affinity microcolumns for microwells (e.g., general affinity microcolumns, ion-exchange affinity microcolumns). In some embodiments, enrichment may be performed via affinity beads in microwells. Affinity beads for microwells may be barcoded with specific barcodes. In some embodiments, affinity microcolumns or beads for post-translational modifications or any other engineered modifications are used for protein enrichment.

### Context C. Denatured Protein Peptide Samples in Droplets in a Microfluidic System

In some embodiments, denatured and reduced protein samples from single cells in droplets in a microfluidic system (Step 1, Context A) or fragmented peptide samples in droplets (Step 1b, Context C), or other form of protein and peptide samples in droplets are subject to protein enrichment. Such samples are enriched to provide purified protein or peptide samples from individual cells in droplets along with barcoded beads in a solution suitable for barcode attachment. In some embodiments, protein enrichment is performed via affinity beads (with barcodes) for droplets. Such affinity beads may be barcoded with cell specific barcodes, spatial barcodes, or other forms of barcodes. In some embodiments, proteins are retained in hydrogel droplets and thus enriched into purified proteins or peptide samples from individual cells in droplets along with barcoded beads in a solution suitable for barcode attachment.

### Context D. Spatially Preserved Protein Peptide Samples (Hydrogel)

The processing of intact samples in Step 1 results in enriched protein/peptides labeled with barcodes. Therefore, spatial samples can proceed to the end of Step 3.

### Step 2b: Protein Preprocessing

### Chemistries for Modifying and Preserving Amino Acids and Post-Translational Modifications (PTMs) for Enhanced Detection.

Example chemistries for modifying and preserving amino acids and post-translational modifications (PTMs) for enhanced detection may include: (a) alkylation of cysteines; (b) phosphorylation tagging and preservation; (c) blocking of lysine side chains with phenyl isothiocyanate (PITC); (d) de-blocking terminal ends for protein sequencing; (e)N-terminal modifications; (f)C-terminal modifications. (g) epitope tags on amino acids or PTMs. Such chemistries are further described below.

Example 1: Alkylation of Cysteines. Cysteines, having been reduced (Step 1), are now treated with iodoacetate or chloroacetate based alkylating agents, or other compatible alkylating agents to create a stable adduct.

Example 2: Phosphorylation tagging and preservation. In some embodiments, the amino acids are modified using phosphorylation tagging and preservation. This chemistry involves replacing phosphorylation sites with stable thiol base tags. For further information, see Knight, Z., Schilling, B., Row, R. et al. Phosphospecific proteolysis for mapping sites of protein phosphorylation. Nat Biotechnol 21, 1047-1054 (2003). https://doi.org/10.1038/nbt863.

Example 3: Blocking of Lysine side chains with Phenyl Isothiocyanate (PITC). In some embodiments, the amino acids are modified using PITC to block lysine side-chains. For N-terminal degradation chemistries, the side chain of lysine residues can potentially interfere, and thus would need to be blocked. Lysine side chains can be blocked by treating proteins/peptides with phenyl isothiocyanate (which reacts to the side chain of lysine residues as well as the N-terminus). Once lysine side chains have been blocked, the N-terminus is exposed by carrying out a single round or cycle of Edman degradation (e.g., add anhydrous TFA to cleave and expose the N-terminus end).

Example 4: applying epitope tags on amino acids so that binding agents towards the epitope or the epitope-amino acids complex can be used to identify the amino acid. In some embodiments, the epitope tags are fluorescent. In some embodiments, the epitope tags are peptides. In some embodiments, the epitope tags are haptens. In some embodiments, the epitope tags are nucleic acids. In some embodiments, the epitope tags are polymers. In some embodiments, the epitope tags are chemical moieties. In some embodiments, the epitope tags are attached with reactive chemicals. In some embodiments, the epitope tags are attached enzymatically. Once the epitope-tagged amino acid is cleaved from the terminal end of the protein, it can be identified with various reagents. Once the epitope-tagged amino acid is cleaved from a peptide, the molecule as a whole is called the “epitope-tag-amino-acids-complex”. In some embodiments, the epitope tags or the epitope tag-amino acids complex are identified with antibodies. In some embodiments, the epitope tags or the epitope-tag-amino-acids-complex are identified with proteins. In some embodiments, the epitope tags or the epitope-tag-amino-acids-complex are identified with aptamers. In some embodiments, the epitope tags or the epitope-tag-amino-acids-complex are identified with somamers. In some embodiments, the epitope tags or the epitope-tag-amino-acids-complex are identified with nucleic acids. In some embodiments, the epitope tags or the epitope-tag-amino-acids-complex are identified with polymers. In some embodiments, the epitope tags or the epitope-tag-amino-acids-complex are identified with nanopores.

In certain instances, after terminal amino acid modifications have been made, but before sequencing is performed, these modifications can be removed to avoid blocking reagents used for protein sequencing.

Natural post translational modifications occur on the N- and C-termini of proteins which would normally block reagents used for protein sequencing. These modifications would need to be removed first before proceeding with sequencing. These modifications can be removed through enzymatic or chemical strategies known to the art. Example N-terminal modifications include acetylation, formylation, methylation, and pyrrolidone carboxylic acid modifications. Acetylation modifications may be removed with acyl peptide hydrolase or acid treatment (1N HCl). Methylation may be removed using aminopeptidases. Formylation modifications may be removed, for example, using acid treatment (e.g., 0.6M HCl treatment). Pyrrolidone carboxylic acid (PCA) may be removed with pyroglutamate aminopeptidase. Example C-terminal modifications may include amidation and methylation, both of which may be removed using carboxypeptidases.

### Step 3: Protein/Peptide Barcode Attachment

Following enrichment, proteins and peptides are tagged with barcodes that uniquely identify each molecule. These barcodes will be used during the protein sequencing steps to link detected amino acids to proteins and peptides. The barcodes used can be derived from a wide range of biological materials that enable the storage and readout of information.

Barcodes may be designed using DNA or RNA. For example, DNA or RNA containing UMI, protein/peptide barcodes, or cell barcodes (e.g., barcodes redundant in Hamming space) may be employed. Barcodes may also be designed using DNA or RNA with hairpin protection, such that the hairpin segment of the barcode will prevent non-specific binding. Alternatively, barcodes may be designed using artificial or modified nucleic acids (locked nucleic acids (LNA) and protein nucleic acids (PNA), hexitol nucleic acids (HNA), cyclohexane nucleic acids (CeNA)) or mixtures thereof. In other embodiments, barcodes are designed using proteins (e.g., Tal Effector, Cas9, Argonaut, Coiled Coils). In other embodiments, chemical polymers are used for barcodes using heavy metal tags.

Chemistries for Attachment of Barcodes or Barcode Primers to Peptides. A range of chemistries exist for attaching barcodes or primers to proteins/peptides that use the intrinsic properties of proteins/peptides, including any conjugation chemistries known in the art. Attachment can be performed via internal attachment, C-terminal attachment, or N-terminal attachment of barcodes to peptides. For example, C-terminal attachment may comprise amide coupling to C-terminus carboxylic group or Photoredox tagging of C-terminus carboxylic group may be used. N-terminal attachment may comprise amide coupling to N-terminus amine group, where specific attachment can occur at low pH, or using 2-pyridinecarboxaldehyde variants for specific attachment to N-terminus are example options. Internal attachment may comprise, for example, (a) amide coupling using EDC/NHS chemistry or DMT-MM to Glutamate or Aspartate; (b) alkylation or disulfide bridge labeling of cysteines; or (c) amide coupling to lysine residues.

### Chemistries for Affixing Proteins and Peptides to Surfaces

Proteins and peptides can also be directly affixed onto surfaces, such as microbeads and slides. One option for surface attachment of proteins can use chemical reagents. In some examples, C-terminal attachment of a polypeptide to a surface may comprise amide coupling to an amine-functionalized surface, or photoredox attachment of the C-terminal end to a surface. N-terminal attachment may comprise amide coupling of the N-terminus amine group to a carboxylic group functionalized surface, or using 2-pyridinecarboxaldehyde variants for specific attachment. Attachment of polypeptide via internal residues to surfaces may use (a) amide coupling using EDC/NHS chemistry or DMT-MM to Glutamate or Aspartate; (b) alkylation or disulfide bridge labeling of cysteines; or (c) amide coupling to lysine residues. Even more, another set of options for surface attachment of proteins can use enzymes. Surfaces labeled with enzyme specific target peptide sequences can be used to attach proteins and peptides using enzymes such as Sortase A, subtiligase, Butelase I, and trypsiligase. These enzymes can be used to attach either the N-terminal or C-terminal end of proteins depending on the target peptide sequences present on surfaces. In some examples, ubiquitin ligase can be used to attach ubiquitin proteins with linker moieties to surfaces. These linker moieties can then be used to chemically attach proteins to ubiquitin on surfaces.

### Context A. Attachment of Protein Peptide to Barcode on a Microbead

In Context A, proteins and peptides collected in bulk solution are tagged with barcodes on a microbead support. In embodiments where proteins or peptides are attached to a barcode on a microbead, purified protein/peptide samples are prepared in suitable buffer for barcode attachment either in bulk solution (Step 2, Context A), in microwells (Step 2, Context B), or in droplets (Step 2, Context C). For single cell samples in microwells or droplets, microbeads bearing protein/peptide barcodes as well as cell-specific barcodes can be introduced here or in Step 1 or Step 2.

Attachment of proteins or peptides to barcodes on a microbead may be via direct ligation of proteins or peptides to barcodes. For example, microbeads may possess peptide and cell barcodes that can react to proteins and peptides. In some embodiments, the barcode may attach to the C-terminus of the protein or peptide. In some embodiments, the barcode may attach to the N-terminus of the protein or peptide. In other embodiments, the barcode may attach to an internal location of the protein.

Attachment of peptides or proteins to a barcode may be performed via indirect association via beads (protein and barcode are both attached to a microbead, but not to each other). In such scenarios, microbeads have reactive sites for proteins or peptides adjacent to barcodes. Attachment may occur via attachment of the C-terminus of proteins/peptides to the bead, attachment of the N-terminus of proteins/peptides to beads, or internal attachment of the protein to a bead.

Attachment of peptides or proteins to barcodes on the bead may result in proteins or peptides tethered to barcodes on microbeads, either in context of bulk solution, microwells, or droplets. Alternatively, proteins/peptides tethered to microbeads bearing barcodes may occur in the context of bulk solution, microwells, or droplets.

### Context B. Attachment of Protein Peptide to Barcode on a Surface

In this context, enriched protein/peptide samples are tagged with barcodes and attached on a bulk surface support, such as a glass slide, a flow cell, or the bottom of well plates.

In some embodiments, attachment is performed using purified protein/peptide samples in suitable buffer for barcode attachment, either in bulk solution (Step 2, Context A) or in microwells (Step 2, Context B). For single cell samples in microwells, the bottom surface of the microwells possess cell-specific and protein barcodes. Attachment results in proteins or peptides tethered to barcodes on surfaces (e.g., glass slide or bottom of microwells or in peptides or proteins tethered adjacent to barcodes on such surfaces).

Attachment of protein/peptide to barcode on a surface may occur via direct ligation of a protein/peptide to a barcode on a surface (e.g., glass slide, or bottom of microwell). Barcodes tethered to surface can directly react to proteins/peptides. Attachment may also occur via indirect association protein/peptide to barcode via proximity on surface (e.g., glass slide, or bottom of microwell). In this scenario, barcodes are tethered to the surface, and there are reactive sites present on the surface for proteins/peptides. With either direct ligation or indirect association, attachment may occur via C-terminus attachment to the bead, via N-terminus attachment of the protein to the bead, or via internal attachment of the protein to bead.

### Context C. Attachment of Protein Peptide to Barcode in Solution (Bulk, or Microwells)

In cases where attachment is performed in solution, such as when using purified protein/peptide samples in suitable buffer for barcode attachment either in bulk solution (Step 2, Context A), in microwells (Step2, Context B), attachment results in peptides or proteins conjugated to their respective barcodes and suspended in solution. Attachment may occur via the C-terminus attachment of proteins/peptides to barcodes, N-terminal attachment of proteins/peptides to barcodes, or internal attachment to barcodes.

### Context D. Samples Consisting of Spatial Information

At this stage, spatial samples (Step 1, Contexts D and E; Step 2, Context D) will have already been processed such that proteins and peptides are tagged with barcodes with unique protein identifiers as well as spatial barcodes (see Step 1). These barcoded proteins from such samples will either be in bulk solution, or on microbeads. These samples will then proceed to Step 4 without additional processing.

### Single-Barcoding Embodiments

The above discussed approaches can be conducted with either a single or multiple attachment(s) to a peptide. For single attachment, we provide possible examples to create a single attachment points on a peptide or protein.

In one approach, lysines and N-terminal amine groups can be labeled with Edman's reagent (PITC). N-terminal based cleavage via a single round or cycle of Edman degradation exposes a single reactive N-terminal amine group. A protease may also be used to cleave the N-terminal amino acid of a peptide to cause a single free N-terminal amine group to be exposed. The single N-terminal amino acid with a free amine group is now a reactive moiety for a single attachment site using amide coupling, aldehyde based, or other similar chemistries.

Carboxylic groups present on aspartic acids, glutamic acids, and C-terminal amino acids can be labeled with a C-terminal sequencing reagent, such as isothiocyanate and similar reagents. Cleavage of the C-terminal amino acid via a single round or cycle of C-terminal sequencing degradation exposes only a single reactive carboxylic group at the C-terminal amino acid. A protease may also be used to cleave peptide at the C-terminus to expose a single free C-terminal carboxylic group. The single C-terminal carboxylic group is now a reactive moiety for a single attachment site.

In another approach, single site labeling on a peptide or protein can be achieved using the specific reactivities of the N-terminus amine group. Amine-based reactions, such as amide coupling, can be carried out at low pH where only the N-terminal amine group is active. In addition, 2-pyridinecarboxyaldehyde and variants can be used to react to the N-terminal amine group.

Site-specific attachment to the carboxylic group of the C-terminus of proteins and peptides can be achieved using the distinct oxidative potential of this carboxylic group. Photoredox reactive can be carried out at potential where only the c-terminal carboxylic groups are decarboxylated, yielding a reactive radical. A variety of electrophilic tags can be reacted to this reactive, decarboxylated C-terminus.

Single-labeling of peptides with barcodes can be achieved by first labeling proteins with multiple barcodes on multiple sites, such as reactive side chains of amino acids. Then, these multi-labeled proteins can be fragmented chemically or using proteases to generate peptides carrying on average a single barcode.

Alternatively, proteins can be fragmented using endoproteases that cut at a specific amino acid, such as trypsin, Lys-C, or Glu-C among others. Once fragmented, peptides can be labeled at the amino acid sites targeted by the endoproteases (e.g., Glutamate for Glu-C, or lysine for Lys-C). Chemicals, such as cyanogen bromide, can also be used to fragment proteins into peptides with specific N or C-terminal amino acids to be used for single site labeling.

Proteins can be prepared with engineered or unnatural amino acids for site-specific labeling. Such engineered amino acids can be introduced at the stage of protein synthesis in cells or tissues. Such engineered amino acids may contain bioorthogonal reactive groups, such azide or alkyne groups among others. Even more, proteins can be prepared with a sequence of amino acids that can be recognized by enzymes that act upon such sequences, such as ligases and sortases.

### Example Multi-Barcoding Embodiments

For multiple attachment, peptides typically have multiple groups available and thus multiple attachment can be achieved using standard chemistry or enzymatic methods known to the art. However, as barcode attachment is stochastic (i.e., the precise barcode and amount of barcodes on each peptide is unknown), having multiple barcodes on a peptide would require a method to associate the various barcodes labeled to that single peptide. Accordingly, in some embodiments of the present disclosure, herein provided are schemes for the association of multi-barcodes on a single peptide. In addition, we also describe an embodiment to obtain multiple of the same barcode to a single peptide.

Methods and systems herein may allow for multi-barcoding via (I) multi-barcoding of proteins and determination of proximity between barcodes, or via (II) bridge amplification to label a protein multiple times with the same barcode sequence. Multi-barcoding may begin with an input step comprising purified proteins in any context (i.e., on a bead surface, on a bulk surface, or in solution).

(I) Multi-Barcoding of Proteins and Determination of Proximity Between barcodes. In this approach, once a protein has been labeled with multiple barcodes, proximity-based polymerase extension is used to copy and associate the sequence of adjacent barcodes. Methods using the approach described above may comprise tagging proteins with a diverse set of barcodes using a combination of internal as well as terminal labeling strategies. Such tagging may be performed using a high barcode concentration to achieve multiple barcodes being attached to each protein. Each barcode comprises a segment for a primer binding site, to which a dual primer linker sequence comprising two adjoined primers that bind to nearby primer binding sites on nearby barcode is added. Once the dual primer linker sequence is added, systems and methods herein may add one or more of a polymerase to extend and copy the barcode sequences of adjacent barcodes. Methods further comprise removing the dual primer linker sequence, which now has copies of adjacent barcodes. After removing the dual primer linker sequence, at this point, the protein with multiple barcodes has the option to be fragmented into peptides each with a single barcode on average. Accordingly, the output of this step may comprise proteins with multiple barcode sequences per protein. The output may further comprise an adjacency matrix of barcode sequences. Alternatively, the output of this step may comprise peptides with individual barcodes generated from fragmenting multi-labeled proteins where the adjacency information between barcode sequences is known.

(II) Bridge amplification to label a protein multiple times with the same barcode sequence. Methods and systems described herein may also perform multi-barcode labelling using bridge amplification. In such a case, a protein is tagged at multiple sites with primer sequences. Then a single barcode is added per protein. That barcode is copied to adjacent primer sequences via bridge amplification. Methods using bridge amplification to label a protein multiple times may comprise: (a) tagging proteins with many copies of a short primer sequence; (b) adding sparse barcode sequences such that only one primer sequence per protein is extended by polymerase extension; (c) carry out bridge amplification where the single extended barcode per protein is copied to adjacent primers, resulting in proteins with multiple primer tags bearing the same barcode sequence per protein; and, if needed, (d) fragmenting these protein sequences to yield peptides with single barcode sequences on average. Accordingly, in some cases, the output of this step comprises peptides with individual barcodes generated from fragmenting multi-labeled proteins where peptides from the same protein have the same barcodes.

### Step 4: Barcode Transfer Reagent (BTR) Attachment

The Barcode Transfer Reagent (BTR) reacts to the termini or internally to peptides and proteins for the sequential removal of amino acids. This reagent may contain at least three components. One possible component is a DNA sequence containing cycle information. In principle, a variety of molecules can be used as barcodes, such as DNA, RNA, HNA, CeNA, proteins, synthetic molecules, or other materials. The second possible component is a primer that hybridizes to a site on the peptide barcode (this is specific to DNA/RNA/HNA/CeNA). Another component is a chemical moiety that conjugates to either the N-, C-, or both terminal ends of a peptide.

Upon addition of the BTR to the barcoded peptides, the reagent will conjugate covalently to either the N- or C-termini of the peptide, depending on its design, as shown in FIG. 4. Other barcode components that may be included can contain: primer sites for NGS sequencing, spacers, restriction sites, and additional barcodes.

Barcode Transfer Reagents may have various reactive moieties. N-terminal reactive molecules that can be used as moieties include, for example Phenyl Isothiocyanate (PITC), ClickP compounds (as described in U.S. Pat. No. 11,499,979, which is incorporated by reference herein in its entirety), dinitrofluorobenzene (DNFB), dansyl chloride, and derivatives or analogs thereof. C-terminal reactive groups include isothiocyanate, thiocyanate and reagents used in Bergman Degradation sequencing as well as analogs. The barcode may be designed using DNA or RNA. Such a barcode may contain a cycle barcode and a primer, or a toehold design. For barcodes designed using proteins, examples may include Tal effector, Cas9, Argonaut, Coiled Coil. Where chemical polymers are used, Mass Spec heavy metals may be used.

Conjugation chemistry of barcode to reactive moiety may involve, for example, click chemistry, thiol chemistry, amino chemistry, or any other conjugations chemistry. Attachment of BTR to terminal amino acids may occur via a variety of mechanisms. For example, proteins may be tethered to barcodes on microbeads or they may be tethered to adjacent to barcodes or other surfaces. Attachment may be performed in solution, such that the barcoded proteins are suspended in solution.

### Context of BTR Attachment to Terminal Amino Acid on a Barcoded Protein/Peptide

### Context 1: Attachment of BTR to Barcoded Protein on a Microbead

Input: Proteins tethered to barcodes on microbeads (or proteins/peptides tethered adjacent to barcodes), either in context of bulk solution, microwells, or droplets (Step 3, Context A and Context D). To attach the BTR to the barcoded protein on a microbead, BTR solution is first added to microbeads with barcoded proteins, and then incubated. Once the reaction has completed, a magnet or centrifuge, for example, may be used to separate beads from the reaction. This is followed by a washing step, resulting in barcoded proteins on microbeads with BTR attached to a terminus on protein.

### Context 2: Attachment of BTR to Barcoded Protein on a Surface

In some embodiments, proteins are tethered to a barcoded protein on a solid support. BTR solution is added to the surface with barcoded proteins. The reaction is incubated and, once complete, a wash is performed to remove unreacted BTR This process leads to barcoded proteins on solid supports with BTR attached to a terminus on protein.

### Context 3: Attachment of BTR to Barcoded Protein in Solution (Bulk or Microwells)

In some embodiments, proteins are conjugated to their respective barcodes and suspended in solution (Step 3, Context C or Context D). In such embodiments, BTR is added to solution containing barcoded proteins. This is then incubated. Reacted barcoded proteins are purified and isolated. This may be performed, for example, via size exclusion columns or affinity columns, resulting in an output of BTR attached to a terminus on each barcoded protein in solution.

### Context 4: Attachment of Barcoded Protein to BTR on a Surface or Microbead

In some embodiments, proteins are conjugated to their respective barcodes and suspended in solution (Step 3, Context C). Microbeads or a solid support are prepared with tethered BTR molecules. In such embodiments, barcoded proteins are added to the microbeads or the solid support containing BTR. Incubation follows, and unreacted barcoded proteins are then removed. The microbeads or solid support are washed, resulting in barcoded proteins attached via a terminus to BTR tethered to a microbead or surface.

### Step 5: Transferring Protein Barcode to BTR

This step comprises transferring barcode information. In some embodiments, the protein barcode is transferred to the BTR, while in other embodiments, the BTR barcode is transferred from the BTR to the protein barcode. FIG. 5 provides a scheme for transfer of barcode information from the BTR reagent to the protein barcode using ligation and polymerase extension steps. FIG. 19 also shows a scheme for transferring protein barcode information to a BTR. In this example embodiment, the protein barcode contains repeats, which are then transferred via ligation and cleavage to a BTR that binds to the N-terminus of the protein. This process is iteratively carried out in sequencing the protein. Other embodiments may comprise a BTR that binds to the C-terminus of the protein.

Transfer methods that may be employed to transfer the protein barcode to the BTR (or to transfer the BTR barcode to the protein barcode) may include polymerase extension, ligation (and cleavage), recombination, or toehold mediated strand displacement and ligation. For polymerase extension, the primer site on BTR binds to a complementary region on a protein barcode. Polymerase is added (e.g., Klenow polymerase, KOD polymerase, TgoT polymerase, or variant thereof), which extends the primer of BTR by copying the peptide barcode onto it. This results in the BTR containing a copy of the peptide barcode. Where ligation and cleavage are used, the peptide barcode and the BTR attached to a terminus are ligated via a ligase. A restriction enzyme is then used to cleaved causing a portion of the peptide barcode to be attached to the BTR. Notably, these approaches can also be used to copy/transfer sequences from the BTR and append them to the protein barcode sequence, where the protein barcode sequence over many round or cycles becomes appended with the sequences of BTR with which it interacted. In addition, polymerase chain reaction (PCR) and isothermal amplification methods may be used to copy the sequences of interacting BTRs and protein barcodes as amplicons (amplification products) that can be collected and analyzed.

### Context 1: Attachment of BTR to Barcoded Protein on a Microbead

In embodiments where the BTR is attached to the barcoded protein on a microbead, step 5 comprises adding reagents necessary for the transfer method (see above) to microbeads containing BTR attached to a barcoded protein tethered on a microbead. Next, the reaction is incubated. When transfer is done, a magnet or a centrifuge may be used to collect microbeads and wash away reagents.

### Context 2: Attachment of BTR to Barcoded Protein on a Surface

In embodiments where the BTR is attached to the barcoded protein on a surface, step 5 comprises adding reagents necessary for transfer method (see above) to a surface (e.g., glass slide) containing BTR attached to a barcoded protein tethered to the surface. The reaction is then incubated and, when transfer is done, reagents are washed away.

### Context 3: Attachment of BTR to Barcoded Protein in Solution

In embodiments, where the BTR is attached to a barcoded protein in solution, step 5 comprises adding reagents necessary for the transfer method (see above) to solution containing BTR attached to a barcoded protein. Next, the reaction is incubated. When transfer is done, the BTR-protein barcode complex is purified using, for example, size exclusion columns or electrophoresis.

### Step 6: Cutting of Terminal End Amino Acids Containing BTR

Cutting of terminal end amino acids containing BTR may be performed via a variety of methods. For example, one option is chemical cleavage, such as acidic cleavage, Edman degradation (PITC, derivatives thereof), or mild basic conditions. For example, in an acidic cleavage, anhydrous TFA may be used for Edman degradation. Examples of basic cleavage may include using triethylamine (for Edman or Thiocyanate degradation) or using KOH for thiocyanate degradation. Alternatively, enzymatic cleavage may be performed using edmanase, aminopeptidases (e.g., Pfu Aminopeptidase I), carboxypeptidase Y (C-terminal sequencing), or acyl peptide hydrolase. Catalytic cleavage may also be used.

It should be noted here that while the primary design of this cleavage step is intended to release the terminal amino acid, it is also possible to design enzymatic cleavage approaches to liberate terminal amino acids as dipeptides or tripeptides, where two, three or more amino acids are released as small peptides.

### Context 1: BTR Attached to Barcoded Protein on a Microbead

In this context, proteins/peptides with molecular barcodes are attached to microbeads with multiple barcoded proteins/peptides per microbead. The Barcode Transfer Reagent (BTR) has reacted to exposed termini. Upon cleavage, the BTR along with the terminal amino acid is then released into solution.

### Context 2: BTR Attached to Barcoded Protein on a Surface

In another possibility, proteins/peptides with molecular barcodes are attached to a large physical surface, such as a glass slide, with multiple barcoded proteins/peptides over a given area. The BTR has reacted to exposed termini. Upon cleavage, the BTR along with the terminal amino acid is then released into solution.

### Context 3: BTR Attached to a Barcoded Protein in Solution

In addition, barcoded protein/peptides are dissolved in solution without a solid support, and the BTR will have reacted to exposed termini. Upon cleavage, the BTR along with the terminal amino acid is then released into solution.

### Context 4: BTR Attached to a Barcoded Protein in a Droplet

In this context, barcoded protein/peptides are prepared in water-oil emulsion droplets with a single barcoded protein/peptide per droplet. The BTR is reacted to the exposed terminus for each molecule. Upon cleavage, The BTR along with the terminal amino acid is then released into solution.

### Context 5: BTR Attached to a Barcoded Polypeptide in a Hydrogel

In this context, barcoded proteins/peptides are covalently tethered in a hydrogel, such as a polyacrylamide or a polyacrylate hydrogel. The BTR is reacted to the exposed terminus for each molecule. Upon cleavage, The BTR along with the terminal amino acid is then released into solution.

Step 7: Collecting BTR with Cleaved Amino Acids

The cleavage of the BTRs liberates the BTR-terminal amino acid complexes (BTR-AC) from the peptide-barcode constructs and releases them into solution.

Context A: BTR-ACs Released into Solution

BTRs released into solution may be collected via a microwell. For example, in the case of microbead attached and barcoded proteins, magnetic separation of beads from supernatant may be performed, and then supernatants from different wells containing BTR-ACs may be transferred and pooled. In another example, in the case of surface attached and barcoded proteins, the supernatant containing BTR-ACs is removed and pooled. A further example may use an affinity column purification method for collecting BTR with cleaved amino acids.

Alternatively, BTRs released into solution may be collected in bulk solution. For example, for surface attached barcoded proteins, supernatant containing BTR-ACs may be removed. Alternatively, affinity column purification may be employed to perform step 7.

Context B: BTRs Released into Droplets

In some contexts, BTR-ACs may be released into droplets. In the case of microbead or microgel attached and barcoded proteins prepared in emulsified droplets, the emulsion is first broken, beads are pulled down, and then separated from supernatant containing BTR-ACs.

### Step 8

### Step 8: BTR Pooling and Sorting

Once cleaved from proteins/peptides, the released Barcode Transfer Reagent-terminal Amino Acid Complexes (BTR-ACs) are identified via affinity binding agents designed to recognize and bind different types of amino acids, PTMs, peptides, or epitope tagged amino acids. These binding agents are used to sort the BTR-ACs from different peptides and sequencing steps according to the identity of the cleaved amino acids.

### Design of Affinity Binding Agents

One option for affinity binding agents is to employ amino acid specific antibodies or nanobodies. For example, these can be raised in animals or evolved in vitro against amino acid targets.

Another option is to employ modified amino acyl t-RNA transferases (synthetases). These enzymes are capable of recognizing individual amino acids. They can be modified to eliminate enzymatic activity and improve binding affinity.

A third option employs artificial protein domains, such as fibronectin domains and others that can be evolved via display approaches to bind amino acids. A further option employs aptamers or somamers, using nucleic acid binding agents and/or their analogs that can be evolved to bind amino acids via SELEX. Alternatively, linking of existing binding agents may be employed. For example, combination of the above binding agents or other binding agents can be linked to bind a range of amino acids dictated by the individual binding agents. linking can be designed such that binding domains from each binding agent are concatenated or linked. These combinatorial linked binding agents can bind amino acids on the terminal end of peptides, internally, or dissociated from peptides.

Another option includes exopeptidases, such as aminopeptidases and carboxypeptidases, modified to remove their peptidase activity so that they only recognize amino acids. Similarly, endoproteases, such as trypsin and trypsin family proteins, can be modified to remove their peptidase activity to use them as amino acid specific binding agents.

While the primary design of affinity binding agents focuses on generating binding agents that recognize individual amino acids, it is also possible to design binding agents that recognize short peptide sequences, such as dipeptides and tripeptides. In addition, binding agents can be designed to recognize amino acids bearing natural or engineered chemical modifications, epitope attachments, or the amino acid complex as a whole.

### Pooling and Sorting Methods

Binding agents can be deployed in a variety of contexts to sort and pool BTRACs.

Option 1. One option for sorting and pulling is to pull down with affinity binding agents on microbeads. In this step, pooled, cleaved BTR-ACs in solution (from Step 7) and affinity reagents on Microbeads in different containers for different amino acids are used. First, BTR-AC solution is added to one set of beads containing binding agents specific for one or more amino acids. This is then incubated, and beads are magnetically pulled down. Supernatant BTR-ACs are transferred to a well with a different set of beads for another amino acid. This is then incubated, the beads are magnetically pulled down, and the process is repeated as needed. The output of this option comprises wells with pulled-down BTR-ACs by amino acid-specific affinity binding agents.

Option 2. Another option employs proximity ligation between barcoded affinity binding agents and BTR-ACs in solution. To do so, pooled, cleaved BTR-ACs (e.g., DNA or RNA barcodes) in solution (from Step 7) and binding agents with amino acid-specific DNA/RNA barcodes in solution are used (an example of binding agents with amino acid-specific barcodes can include antibodies or nanobodies tagged with DNA barcode indicating their amino acid specificity). A mixture of affinity binding agents targeting different amino acids is then prepared, each with its own amino acid specific barcode. Next, BTR-AC solution is mixed with barcoded affinity binding agents and incubated. After incubation, ligase is added to connect the sequences of bound BTR-ACs and binding agents. Finally, PCR is used to amplify ligated segments of BTR-ACs and binding agent sequences. This results in an output comprising PCR products of BTR-ACs sequences ligated to affinity binding agent barcode sequences.

Option 3. A third option employs proximity ligation between barcoded affinity binding agents on a microbead and BTR-ACs. Such a method uses pooled, cleaved BTR-ACs (e.g., DNA or RNA barcodes) in solution (From Step 7) and affinity binding agents with amino acid specific DNA/RNA barcode on a microbead. First, a mixture of affinity binding agents on microbeads targeting different amino acids is prepared, each with its own amino acid specific barcode. BTR-AC solution with barcoded affinity binding agents is added. The mixture is incubated, and after incubation, ligase is added to connect the sequences of bound BTR-ACs and binding agents. PCR may then be employed to amplify ligated segments of BTR-ACs and binding agent sequences. This produces a ligation product containing BTR barcode and affinity binding agent barcode PCR products of BTR-AC sequences ligated to affinity binding agent barcode sequences.

Option 4. A further option may employ proximity-based polymerase information transfer between barcoded affinity binding agents on a microbead and BTR-ACs., this option uses pooled, cleaved BTR-ACs (e.g., DNA or RNA barcodes) in solution (from Step 7) and affinity binding agents with amino acid-specific DNA/RNA barcode and primer on a microbead. First a mixture of affinity binding agents on microbeads targeting different amino acids, each with its own amino acid specific barcode, is prepared. Next, BTR-AC solution is added to the barcoded affinity binding agents. The mixture is incubated, and after incubation, the primer segment on the barcode of the affinity binding agent will be extended via a polymerase to copy the BTR-AC information. The output of this option comprises a polymerase extension product on affinity binding agent bearing microbeads containing BTR-AC barcode as well as affinity binding agent barcode.

### Step 8B: BTR Signal Amplification

The accuracy of the ex-situ analysis can be enhanced by repeatedly interrogating the same barcode-amino acid complex (i.e., BTR-AC). In order to do so, one option employs iterative binding between affinity binding agents and BTR-AC, ligation, followed by PCR. Here, pooled, cleaved BTR-ACs (e.g., DNA or RNA barcodes) in solution (from Step 7) and affinity binding agents with amino acid specific DNA/RNA barcode on a microbead are employed. First, a BTR-AC transiently binds to the affinity binding agent on a microbead. Upon binding, the BTR segment ligates to the barcode of the affinity binding agent. Next, PCR amplifies the ligated portion copying the BTR sequence and binding agent barcode sequence. Following PCR, the BTR-AC unbinds from the binding agent and the cycle repeats. This results in PCR products containing a BTR barcode sequence ligated to binding agent barcode.

Another option for enhancing accuracy is through iterative binding between affinity binding agents and BTR followed by transcription amplification. Such an option employs pooled, cleaved BTR-ACs (e.g., DNA or RNA barcodes) in solution (from Step 7) and affinity binding agents with amino acid specific DNA barcode containing a T7 RNA polymerase transcription site, on a microbead. First, a BTR-AC is allowed to transiently bind to the affinity binding agent on a microbead. Upon binding the BTR segment ligates to the barcode of the affinity binding agent. Next, a T7 Polymerase is added, which binds to the promoter sequence on the binding agent barcode, to transcribe and copy the ligated sequences of the binding agent and BTRAC. Following transcription, the BTRAC unbinds from the binding agent and the cycle repeats. The output of this option comprises transcription products containing BTR barcode sequences ligated to affinity binding agent barcodes.

Multi-read via a nanopore: The BTR-amino acid complexes (BTR-ACs) produced during the protein sequencing operations can be read out using nanopores. Nanopore can include biological transmembrane channels and proteins such as MspA and aerolysin nanopores among others. Nanopores can also be constructed from solid state materials, such as silicon nitride nanopores or carbon nanotube or graphene-based substrates. When BTR-ACs are translocated through these nanopores, a change in current flowing through the nanopore occurs consistent with the identity of the amino acid. This change in current can be measured though a current or voltage measuring apparatus. BTR-ACs may be modified with DNA, protein, or polymeric based handles to facilitate their delivery, translocation, and identification through these nanopores. Furthermore, the use of these nanopore can provide information of the barcode sequences present in the cleaved BTR-ACs. Therefore, nanopore based readout can provide information on the identity of amino acids as well associated barcode sequences. It is also possible to read the identity of a BTR-ACs in a nanopore multiple times by repeatedly presenting the same BTR-AC molecule to the nanopore and measuring changes in current. This may be achieved by using helicases, such as Hel308, that pull BTR-AC molecules back and forth in nanopores enabling re-reads.

### Step 9: BTR-AC Readout

At this stage, BTR-ACs have been sorted according to amino acid identity, or the interaction between BTR-ACs and barcoded affinity binding agents in Step 8 has produced ligation or amplification products containing the BTR-ACs sequences as well as the barcodes of affinity binding agents. Therefore, for each BTR-AC, these sequences now contain information on the amino acid identity, peptide/protein of origin, and cycle number, in addition to any primer or other functional sequences. This information can be readout through different approaches.

Option 1. One example option employs Next Generation Sequencing: Illumina Sequencing by Synthesis. The input of this step can comprise BTR-ACs pulled down by amino acid specific affinity binding agents (from Step 8, Option 1). The input may also comprise ligation, polymerase extension, PCR, or transcription products of interaction between BTR-ACs and affinity binding agents (From Step 8, options 2-4, or from Step 8b). In this step, first DNA and RNA BTR-AC sequences and PCR products are processed according to standard library preparation techniques for Next Gen Sequencing. These libraries are then sequenced on an Illumina Sequencer and result in an output of Illumina Sequencing by synthesis reads.

Option 2. Another option employs hybridization based read out, such as SeqFISH, NanoString, or similar variants. In such an option, the input of this step can comprise BTR-ACs pulled down by amino acid specific affinity binding agents (from Step 8, Option 1) or ligation, polymerase extension, PCR, or transcription products of interaction between BTR-ACs and affinity binding agents (from Step 8, options 2-4, or from Step 8b). In this example, the sequences of BTR-ACs as well as ligation and PCR products are readout using sequencing by hybridization approaches. These approaches involve single molecule imaging of immobilized BTR-AC sequences along with repeated hybridization with probes used to determine the target sequences. The output of employing this option may for example comprise microscopy images showing fluorescent hybridization against BTR-ACs or BTR-ACs ligated to affinity binding agent barcodes.

A third option employs Nanopore Sequencing: Oxford Nanopore. The input of this option may comprise BTR-ACs pulled down by amino acid specific affinity binding agents (from step 8, Option 1) or ligation, polymerase extension, PCR, or transcription products of interaction between BTR and affinity binding agents (from step 8, options 2-4, or from Step 8b). In this option, DNA and RNA BTR-AC sequences and PCR products are processed according to standard library preparation techniques for nanopore sequencing. These libraries are then sequenced on a Nanopore sequencer (e.g., Oxford Nanopore devices). The output of this third option is nanopore sequencing reads. Another option employs the readout of peptide-based barcodes. Proteins and peptides to be sequenced can be tagged with peptide-based barcodes. In one case, such peptide-based barcodes involve a combination of epitope tags that can uniquely identify each tagged protein/peptide. Owing to the availability of multiple epitope tags, epitopes can be used in a combinatorial fashion for the multiplexed identification of protein and peptide targets. Peptide barcodes using epitopes can be readout and identified using antibodies that target each epitope. As examples, the readout of such peptide barcodes can be microscopy-based or nanopore-based.

Other options may be employed, as well. For example, mass spectrometry may be employed to generate a BTR-AC read out.

Step 10: Peptide Read Reconstruction from BTR Readout

Once BTR-ACs have been readout and their sequence determined, the next step is to reconstruct the sequences of the peptides from which these BTR-ACs originated. The sequence of a BTR-AC provides the identity of the amino acid, the cycle number in the peptide sequencing round or cycles, and the barcode sequence of the parent peptide. Given an adequate yield in the identification of BTR-ACs, this information is sufficient to computationally reconstruct the sequence of peptides. A de novo approach can be used where this information is used to build the sequences of peptides. Alternatively, the data can be compared to expected results from a proteomic database to infer the identity and sequence of peptides, akin to common data analysis approaches in mass spectrometry proteomics. In either approach, Step 10 begins with NGS sequencing reads (e.g., fastq files) or hybridization-based barcodes (Step 9, Options 1 and 2).

### Step 10, Example A: De-Novo Read Reconstruction

One option employs reconstructing protein sequences from NGS or hybridization based read out. This option comprises first reconstructing barcode sequences from high quality NGS reads. Next, reads from barcoded samples (e.g., single cell or spatial samples) are pooled. Next, for each cleaved BTR with amino acid, a parent protein barcode and sequence round or cycle is assigned. This may be done by either data from pull down of BTR-ACs with affinity binding agents (Step 8, Option 1) or through ligation or polymerase products containing affinity binding agent information as well as BTR-AC sequence (Step 8, Option 2). Next, putative peptide reads are assembled based on parent protein barcode, amino acid identity, and cycle number barcode. The next step is to detect and discard erroneous reads. This may be done through probabilistic modeling of accuracy of reads. This results in reconstructed, fragmentary, peptide sequences (Contigs) with possible gaps for missed or unidentified round or cycles/amino acids.

An alternative option for de novo read reconstruction employs end-to-end, unsupervised machine learning based reconstruction of peptide reads. This option may employ a Machine Learning Algorithm, which refers to a deep-learning based model that takes as its input NGS sequencing reads associated with a parent protein/peptide barcode, and outputs the likely reconstruction of peptide reads (contigs). Training of the model will be conducted with protein sequencing runs using known protein/peptide standards. This step provides reconstructed, fragmentary, peptide sequences (Contigs) with a probability assigned to each amino acid as well as the assembled peptide sequence.

### Step 10, Example B: Reference Based Reconstruction

For known proteome databases, reference-based reconstruction may be performed by simulating NGS reads that would be generated from the set of possible peptides in each database when processed through this experimental workflow. For each possible peptide, the simulation will produce NGS reads mimicking the output of this protein sequence system. Next, the real (experimental) NGS reads from a run are matched to simulated reads from candidate peptides from a database based on likelihood. This results in reconstructed, fragmentary, peptide sequences (Contigs) with probability assigned to the assembled peptide sequence.

These reconstruction approaches are expected to be compatible with other approaches of reading out BTR-AC sequences beyond NGS sequencing.

Step 11: Assembly of Peptide Reads into Protein Sequences

In this step, the peptide contig reads will be assembled into full protein sequences. There exist a range of approaches from the fields of transcriptomics and proteomics that can be brought to bear to the problem of assembling peptide reads into full protein sequences. Reference-based approaches commonly used in Mass Spectrometry Proteomics, such as ProteinProphet, can be leveraged to query for the presence of proteins based on sequenced peptides. In addition, a wide-range of reference-free assembly methods used in transcriptomics can be adapted. Here, we provide two examples for a de-novo and reference-based assembly of peptides.

Step 11, Example A: De-Novo Assembly (K-mer and De Brujin Graph based approach). In this example, reconstructed, fragmentary, peptide sequences (Contigs) from De novo reconstruction (Step 10, Example A) or reference-based reconstruction (Step 10, Example B) are used as samples. First, for each sample, all reconstructed reads are broken down into short k-mer sequences. Next, K-mer sequences from any reads are assembled into longer contig sequences. A De Brujin graph for representing splice variants and post-translational modification is then constructed. Next, the de Brujin graph is traversed and isoforms are assembled. Finally, the expression level of each isoform is determined using a Bayesian approach. As a result of Step 11 a, for each sample (or single cell read), assembled isoforms of proteins and their expression level is provided.

Step 11, Example B: Reference Based Assembly. In step 11b, reconstructed, fragmentary, peptide sequences (Contigs) from De novo reconstruction (Step 10, Example A) or reference-based reconstruction (Step 10, Example B) are used as samples. For a given sample and known proteome database, all peptide reads are mapped to a database. Next, adjacent reads are connected to build sequences of possible isoforms. Finally, the expression level of each isoform is estimated. For each sample (or single cell read), assembled isoforms of proteins and their expression level are determined.

### Step 12: Evaluation of Assembly and List of Proteins and Enrichment

The last step of the workflow nay comprise checking the accuracy and quality of the assembly. Here, a range of established practices from the art can be applied. Standard proteins and peptides that are spiked in along with samples can be used to gauge for errors and artifacts. The quality of the assembly can be checked by comparing results with known proteomic databases for expected errors.

In Step 12, assembled isoforms of proteins and their expression level for all possible samples (Step 11) are evaluated. The first part of Step 12 comprises evaluation and error correction. A de novo assembly evaluation may be used to check for missing segments of sequences; incorrect or redundant assembly; and/or ensure coverage is uniform. A reference-based evaluation may also be employed to compare the assembly to known protein databases. Next, normalization is performed by using spiked—in standards to account for biases between samples. The result of step 12 is a normalized and cleaned up assembly of isoforms of proteins and expression levels for all samples.

TABLE 1 provides potential sources of error, along with example approaches for correcting these errors.

TABLE 1
Potential Sources of Error

| Error | Cause | Effect | Tracking and Correction |
|---|---|---|---|
| Peptide/protein | During protein extraction and | Reads biased towards | Use known biological samples with |
| loss during | purification and/or digestion | abundant proteins | well quantified protein distributions |
| sample preparation | steps, there might be loss of | | to gauge loss; determine lowest copy |
| | proteins | | number detected per cell |
| BTR fails to react | Due to reaction inefficiency, | For a given protein | Use cycle barcode of BTR to keep |
| | BTR fails to react to a | target, this will | track of round or cycles. Every |
| | protein/peptide molecule | appear as a gap in the | peptide should have a read for every |
| | | protein sequence | cycle. If there is a gap, indicates |
| | | | BTR failed to react |
| BTR Inefficiency: | BTR fails to cleave for a given | For a given | Cycle barcode of BTR will indicate |
| Missed sequencing | round or cycle, or multiple | protein/peptide, this | which round or cycles were missed. |
| round or cycles | round or cycles, because of | error will result in | For a given peptide, it is possible to |
| | inefficiencies in the chemistry | missed cycles (i.e., | reconstruct the correct sequence by |
| | | Cycles with no read | comparing to the proteome, even if |
| | | for a particular | there are gaps. |
| | | protein/peptide) | |
| Failure of BTR to | BTR becomes uncleavable | For a given | Rates of termination can be estimated |
| cleave: premature | because of inefficiencies or | protein/peptide, | using standard peptides. Short reads |
| termination of | unexpected side reactions | after termination, | from premature termination events |
| sequencing | | no amino acids are | can still be identified by comparing |
| | | liberated. This will | to the proteome depending on their |
| | | result in a short | lengths |
| | | read for a given | |
| | | peptide. | |
| Failure of | Peptide barcode fails to | The liberated amino | Rate of failure to transfer can be |
| Barcode Transfer | transfer to the BTR due to | acid with BTR cannot | estimated using a simulated sample |
| (Step 5) | inefficiencies in ligation | be associated with a | with standard peptides. During |
| | of polymerase extension | polypeptide. This | sample runs, barcodes can be spiked |
| | | effect acts as a | in during each cycle to keep track of |
| | | missed cycle or | transfer rates |
| | | round or cycle. | |
| Loss of Cleaved | Collection and pooling are | Lost BTRs will | Track lost BTRs via cycle number |
| BTR-amino acid | bound to have inefficiencies, | appear as gaps in | and protein barcode |
| complexes during | and each cleaved BTR molecule | protein sequences | |
| collection and | is unique, so there are | | |
| pooling (Step 7 | bound to be some losses. | | |
| and 8) | | | |
| False positives | Non-specific binding of cleaved | amino acid will be | Repeated measurements of the |
| in amino acid | terminal amino acid with BTR | misidentified as a | interaction between the amino acid |
| identification: | to unintended affinity binding | different amino acid | with BTR and affinity binding agent |
| non-specific | agent | | will provide a probability distribution |
| binding | | | of the identity of the amino acid, |
| | | | which can be used to make a call |
| False negative in | Cleaved terminal amino acid | Loss of the BTR | Cycle barcode of BTR will indicate |
| pull down or | with BTR is lost due to non- | amino acid results in | which round or cycles were missed. |
| affinity binding | specific binding or | a missed cycle for a | For a given peptide, it is possible to |
| agent detection: | inefficiencies from sample | given amino acid | reconstruct the correct sequence by |
| lost BTR amino acid | handling | | comparing to the proteome, even if |
| | | | there are gaps |
| Read out Error | Final readout for the BTR | Barcodes will fail | Error-proof barcodes can be designed |
| | sequence is error prone due to | to be identified, or | that use Hamming distance to |
| | intrinsic errors in method of | might be misidentified | account for possible errors in barcode |
| | sequencing, be it either next | as a different barcode, | readout |
| | gen sequencing, hybridization | resulting either a | |
| | based read out, or mass | meaningless read or an | |
| | spectrometry | incorrect assignment | |
| | | of an amino acid to a | |
| | | protein. | |

### Computer Systems

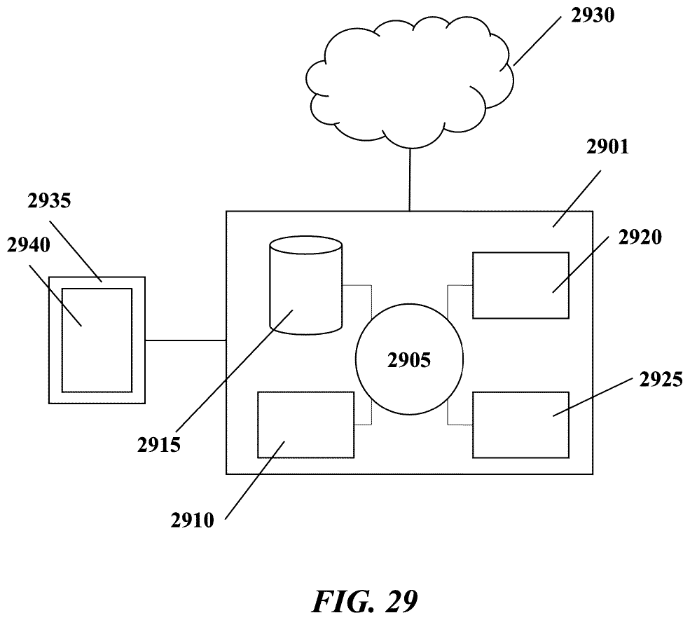

The present disclosure provides computer systems that are programmed to implement methods of the disclosure. FIG. 29 shows a computer system 2901 that is programmed or otherwise configured to process sequencing data of nucleic acid barcode molecules and output protein sequence information. The computer system 2901 can regulate various aspects of automation of operating procedures and processing of data of the present disclosure, such as, for example, automating liquid handling or robotic systems for performing the methods described herein, inputting sequencing reads obtained from DNA sequencing, and outputting a reconstruction of the primary structure of a protein or peptide. The computer system 2901 can be an electronic device of a user or a computer system that is remotely located with respect to the electronic device. The electronic device can be a mobile electronic device.

The computer system 2901 includes a central processing unit (CPU, also “processor” and “computer processor” herein) 2905, which can be a single core or multi core processor, or a plurality of processors for parallel processing. The computer system 2901 also includes memory or memory location 2910 (e.g., random-access memory, read-only memory, flash memory), electronic storage unit 2915 (e.g., hard disk), communication interface 2920 (e.g., network adapter) for communicating with one or more other systems, and peripheral devices 2925, such as cache, other memory, data storage and/or electronic display adapters. The memory 2910, storage unit 2915, interface 2920 and peripheral devices 2925 are in communication with the CPU 2905 through a communication bus (solid lines), such as a motherboard. The storage unit 2915 can be a data storage unit (or data repository) for storing data. The computer system 2901 can be operatively coupled to a computer network (“network”) 2930 with the aid of the communication interface 2920. The network 2930 can be the Internet, an internet and/or extranet, or an intranet and/or extranet that is in communication with the Internet. The network 2930 in some cases is a telecommunication and/or data network. The network 2930 can include one or more computer servers, which can enable distributed computing, such as cloud computing. The network 2930, in some cases with the aid of the computer system 2901, can implement a peer-to-peer network, which may enable devices coupled to the computer system 2901 to behave as a client or a server.

The CPU 2905 can execute a sequence of machine-readable instructions, which can be embodied in a program or software. The instructions may be stored in a memory location, such as the memory 2910. The instructions can be directed to the CPU 2905, which can subsequently program or otherwise configure the CPU 2905 to implement methods of the present disclosure. Examples of operations performed by the CPU 2905 can include fetch, decode, execute, and writeback.

The CPU 2905 can be part of a circuit, such as an integrated circuit. One or more other components of the system 2901 can be included in the circuit. In some cases, the circuit is an application specific integrated circuit (ASIC).

The storage unit 2915 can store files, such as drivers, libraries and saved programs. The storage unit 2915 can store user data, e.g., user preferences and user programs. The computer system 2901 in some cases can include one or more additional data storage units that are external to the computer system 2901, such as located on a remote server that is in communication with the computer system 2901 through an intranet or the Internet.

The computer system 2901 can communicate with one or more remote computer systems through the network 2930. For instance, the computer system 2901 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 2901 via the network 2930.

Methods as described herein can be implemented by way of machine (e.g., computer processor) executable code stored on an electronic storage location of the computer system 2901, such as, for example, on the memory 2910 or electronic storage unit 2915. The machine executable or machine-readable code can be provided in the form of software. During use, the code can be executed by the processor 2905. In some cases, the code can be retrieved from the storage unit 2915 and stored on the memory 2910 for ready access by the processor 2905. In some situations, the electronic storage unit 2915 can be precluded, and machine-executable instructions are stored on memory 2910.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 2901, can be embodied in programming. Various aspects of the technology may be thought of as “products” or “articles of manufacture” typically in the form of machine (or processor) executable code and/or associated data that is carried on or embodied in a type of machine-readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine-readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 2901 can include or be in communication with an electronic display 2935 that comprises a user interface (UI) 2940 for providing, for example, output amino acid sequences, mapped peptide sequences to a peptide or protein database, identity of a peptide or protein, etc. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 2905. The algorithm can, for example, input a DNA sequence (e.g., of the nucleic acid barcode molecules from BTR-ACs described herein) and output an amino acid sequence of a peptide or map a peptide sequence back to a protein database to identify the peptide.

### EXAMPLES

### Example 1. Sample Preparation

### Method 1. Cell Culture Preparation in a Culture Plate

HEK293-FT cells (Invitrogen) are cultured on Nunc Lab-Tek II Chambered Coverglass (Thermo Scientific) in D10 medium (Cellgro) supplemented with 10% fetal bovine serum (FBS) (Invitrogen), 1% penicillin-streptomycin (Cellgro), and 1% sodium pyruvate (BioWhittaker). Cultured cells are grown in 37° C. incubators until reaching 80% confluency.

Method 2. Fresh Frozen Brain Tissue Preparation from Mice

Mice are terminally anesthetized with isoflurane, then decapitated, and the brain dissected out into a cryomold with OCT embedding matrix. The cryomold is then placed in a dry ice/isopentane bath. Overall, freezing of the brain is completed within 5 min after euthanasia. 15 μm slices are then sliced on a Cryotome (Leica) onto a 1 mm glass slide and then immediately stored at −80° C. until use.

### Example 2. Protein Extraction and Preprocessing

General Principle: Once samples have been acquired, proteins can be extracted from their native environment to be processed. This step involves the lysis of cells, either in a cell culture, single cell, or tissue context, and the purification of released proteins from the remaining content of cells. Often at this stage, cysteine amino acids are alkylated to prepare them for downstream protein sequencing steps. Other groups may be protected such as PTMs, but are currently not specified in this example.

Method 1: Extraction, Reduction, and Alkylation of Proteins from Cell Culture Sample

Plated HEK293-FT cells are treated with 0.05% Trypsin (Coming) for 5 minutes at room temperature to detach the cells from the plate. Detached cells in Trypsin solution are diluted in D10 media and centrifuged at 300×g for 5 minutes. Pelleted cells are washed once with DPBS.

Cells are lysed, and extracted proteins are alkylated as described previously (Kulak et al, 2014). Briefly, cells are resuspended in 1% (w/v) sodium deoxycholate, 10 mM TCEP, 40 mM 2-chloroacetamide (CAA), 100 mM Tris, pH 8.5, and lysed by 5 min incubation at 95° C. and sonication for 15 min. Cell debris is pelleted by centrifugation at 13,200 r.p.m. for 5 min and the clarified lysate is transferred into a new vial.

Method 2: Extraction, Reduction, and Alkylation of Proteins from Fresh Frozen Tissue Sample

Fresh frozen tissue sections (5-10 μm) are removed from glass slides and placed in microcentrifuge tubes. Samples are extracted in RIPA buffer (150 mm NaCl, 10 mm Tris, 0.1% SDS, 1% Triton, 1% sodium deoxycholate, 5 mm EDTA, protease inhibitor) and homogenized by ultrasonication (Baganto et al, 2007). Then TCEP and 2-chloroacetamide (CAA) are added to final concentrations of 10 mM and 40 mM respectively, and the sample is incubated at 60° C. for 30 minutes. Tissue debris is pelleted by centrifugation at 13,200 r.p.m. for 5 min and the clarified lysate is transferred into a new vial.

### Example 3. Protein Digestion and Enrichment

General Principle: Proteins extracted from samples can be fragmented into peptides to facilitate downstream sequencing steps. Peptides are smaller and lack the secondary structure of proteins, which might result in unintended protein-protein interactions or inefficient chemical reactions during processing. In addition, fragmenting proteins into peptides provides a redundant approach to querying the presence of a given protein. Though peptides are convenient, this step can be skipped and the workflow carried out with intact proteins. Fragmentation of proteins into peptides can be carried out with enzymes or chemical reagents. At this stage, lysines are treated with phenyl isothiocyanate (PITC) to prevent their side chains from reacting to the sequencing reagent downstream. PITC will also protect terminal ends of proteins, however, this can be removed by conducting one round or cycle of Edman degradation (this step is found later). In addition, cysteine groups are protected with PITC, oxidization, or iodoacetamide from possible breakdown during exposure to Edman associated chemistries.

Method 1. Digestion of Proteins Via Endopeptidase and Protection of Lysine Residues with PITC

Extracted proteins are diluted 1:10 in 1% (w/v) sodium deoxycholate, 100 mM Tris pH 8, and digested by adding endoproteinase Glu-C at a 1:50 protease to substrate protein ratio (w/w) and incubated overnight at 37° C. The digest is acidified with 2% TFA and sodium deoxycholate is extracted using ethyl acetate and vigorous shaking. The organic phase is removed after centrifugation at 13,200 r.p.m. for 5 minutes. The peptides are desalted on C18 StageTips and eluted into 0.5M NaHCO3.

Lysine residues and reactive amines are then protected by adding Phenyl isothiocyanate (PITC). PITC is added to the eluted peptides to reach a final concentration of 10 mM. The reaction is carried out for 30 minutes at 50° C. Finally, the reacted peptides are desalted on C18 StageTips and eluted into 0.1 M 2-(N-morpholino)ethanesulfonic acid (MES).

Method 2. Digestion of Proteins Via Cyanogen Bromide and Protection of Lysine Residues, Terminal Amines and Cysteine with PITC

Extracted proteins are resuspended in 70% formic acid, and then 2 mg of Cyanogen Bromide is added for every 1 mg of protein. The reaction is incubated in the dark overnight. The solvent is then removed and the digested peptides lyophilized using a SpeedVac concentrator. The lyophilized peptides are then resuspended in water, desalted on C18 StageTips, and eluted into 0.5M NaHCO₃.

Lysine residues, the terminal amine, and cysteine residues are then protected by adding Phenyl isothiocyanate (PITC). PITC is added to the eluted peptides to reach a final concentration of 10 mM. The reaction is carried out at 50° C. for 30 min in neural media to target cysteines and later for an additional 30 min in basic pH (10.5) for primary amines. Finally, the reacted peptides are desalted on C18 StageTips and eluted into 0.1 M 2-(N-morpholino)ethanesulfonic acid (MES).

### Example 4. Attachment of Peptides to Molecular Barcodes

General Principle: Peptides are conjugated with molecular barcodes to identify them during post-sequencing analysis. While a variety of molecular barcodes can be used (e.g., nucleic acid based, protein based), the provided example is toward DNA barcodes. A library of DNA barcodes is created capable of uniquely tagging a set of peptides in a given sample. To associate the DNA barcodes to peptides, the barcodes can be conjugated to either N- or C-termini of peptides depending on the mode of sequencing downstream. Since the peptide-barcode constructs will undergo a series of chemical and enzymatic steps downstream, it is convenient to prepare them on a solid support, such as magnetic beads or glass slides, to minimize sample loss and facilitate handling.

### Method 1. C-Terminal Anchoring of Peptides to DNA Barcodes on Magnetic Beads

1 μm magnetic beads bearing DNA barcodes are prepared as follows. DNA barcodes are synthesized bearing unique molecular identifiers (UMIs) as well as random protein barcode sequences (i.e., a set of degenerate bases), which will later be used to identify sequenced peptides. These DNA barcodes possess a 5′ Azide modification and a 3′ amine modification. A slurry of 1 μm magnetic beads with DBCO modifications (Jena Biosciences) is then reacted with DNA barcodes suspended in 1× PBS for 1 hr at a molar ratio of 1:1000-1:1,000,000 (concentrations that yield low likelihood of two DNA sequences next to one another). Magnetic beads are then pulled down and unreacted DNA barcodes are washed away.

A solution of 4 mM of 1-ethyl-3-(3-dimethylamino) propyl carbodiimide, hydrochloride (EDC) and 10 mM N-hydroxysulfosuccinimide (NHS) is prepared in 0.1 M MES buffer and added to the barcoded-bearing magnetic beads. The digested peptides are then added to a final concentration of 10 μM and the mixture is incubated for 4 hours at room temperature. The magnetic beads are pulled down and unreacted peptides are washed away.

At this stage, the N-termini of peptides is blocked by PITC from the lysine protection step. To expose the N-termini, the PITC and the terminal amino acid are cleaved. The beads with the barcoded peptides are first washed with acetonitrile. Then, 100% Trifluoroacetic acid (TFA) is added to the beads. The reaction is incubated for 30 minutes at 50° C. The beads are then washed once with ethyl acetate, twice with water, and then suspended in Coupling Buffer (acetonitrile:pyridine:triethylamine:water at a respective ratio of 5:2:1:3).

### Method 2. C-Terminal Anchoring of Peptides to DNA Barcodes on Treated Glass Slides

To anchor peptides to their DNA barcodes on glass slides, slides are first prepared with polyethylene glycol (PEG) passivation and functionalization with DBCO group as described previously with slight modifications (Bieling et al, 2010). Briefly, standard microscopy glass slides are cleaned by treatment with 3M NaOH and sonication for 5 minutes. The slides are then rinsed with deionized water repeatedly. Slides are then treated with “Piranha” solution (2 volumes of 30% hydrogen peroxide and 3 volumes of 95% sulfuric acid) for 40 minutes in a sonicator bath. After washing with deionized water, glass slides are treated with (3-Glycidyloxypropyl)-trimethoxysilane (GOPTS) for 15 minutes at 75° C. After washing with deionized water, the slides are further treated with DBCO-PEG5K-Amine (Nanocs) for 15 minutes at 60° C. After washing with deionized water, the slides are ready for functionalization with DNA barcodes.

DNA barcodes are synthesized bearing unique molecular identifiers (UMIs) as well as random protein barcode sequences (i.e. a set of degenerate bases), which will later be used to identify sequenced peptides. These DNA barcodes possess a 5′ Azide modification and a 3′ amine modification. Slides are incubated with DNA barcodes at a concentration of 10 μM in 1× PBS for 1 hr, and then washed with 0.1M MES buffer. A solution of 4 mM of 1-ethyl-3-(3-dimethylamino) propyl carbodiimide, hydrochloride (EDC) and 10 mM N-hydroxysulfosuccinimide (NHS) is prepared in 0.1 M MES buffer. The digested peptides are then added to a final concentration of 10 μM and the mixture is added to the glass slides and incubated for 4 hours at room temperature. Slides are then washed with deionized water.

At this stage, the N-termini of peptides is blocked by PITC from the lysine protection step. To expose the N-termini, the PITC and the terminal amino acid are cleaved. Slides with the barcoded peptides are first washed with acetonitrile. Then, 100% Trifluoroacetic acid (TFA) is added to the slides. The reaction is incubated 30 minutes at 50° C. The slides are then washed once with ethyl acetate, twice with water, and then placed in Coupling Buffer (acetonitrile pyridine:triethylamine:water at a respective ratio of 5:2:1:3).

### Method 3. N-Terminal Anchoring of Peptides to DNA Barcodes on Magnetic Beads

1 μm magnetic beads bearing DNA barcodes are prepared as follows. DNA barcodes are synthesized bearing unique molecular identifiers (UMIs) as well as random protein barcode sequences (i.e. a set of degenerate bases), which will later be used to identify sequenced peptides. These DNA barcodes possess a 5′ Azide modification and a 3′ NHS modification. DNA barcodes are suspended in 1× PBS at 10 μM and immediately reacted with 6-(1-Piperazinyl Methyl)-2-pyridinecarboxaldehyde (Sigma) at a final concentration of 100 μM. Reacted DNA barcodes are then purified via gel electrophoresis and suspended in 1× PBS. A slurry of 1 μm magnetic beads with DBCO modifications (Jena Biosciences) is then reacted with DNA barcodes suspended in 1× PBS for 1 hr at a molar ratio of 1:1000-1:1,000,000 (concentrations that yield low likelihood of two DNA sequences next to one another). Magnetic beads are then pulled down and unreacted DNA barcodes are washed away with 1× PBS. Finally, digested peptides (without PITC treatment) are added to the barcode-bearing magnetic beads at a concentration of 10 μM in 1× PBS. After a 4-hour reaction at room temperature, the beads with the barcoded peptides are washed with 1× PBS.

### Method 4. C-Terminal Anchoring of Peptides to DNA Barcodes in Solution

DNA barcodes are synthesized bearing unique molecular identifiers (UMIs) as well as random protein barcode sequences (i.e., a set of degenerate bases), which will later be used to identify sequenced peptides. These DNA barcodes possess a 5′ Azide modification and a 3′ amine modification. Prior to use, DNA barcodes are prepared in 100 μM stock solution in 0.1M MES buffer. A solution of 4 mM of 1-ethyl-3-(3-dimethylamino) propyl carbodiimide, hydrochloride (EDC) and 10 mM N-hydroxysulfosuccinimide (NHS) is prepared in 0.1 M MES buffer. To this solution, protected and digested peptides are added to a final concentration of 10 μM and incubated for 30 minutes. Then, DNA barcodes are added to a final concentration of 20 μM and the reaction is carried out for another hour. Finally, peptide-barcode conjugates are purified using denaturing gel electrophoresis and suspended in Coupling Buffer (acetonitrile:pyridine triethylamine:water at a respective ratio of 5:2:1:3).

### Example 5a. Preparing Barcode Transfer Reagent (Btr)

General Principle: The Barcode Transfer Reagent (BTR) possesses two functionalities. The first is a moiety capable of sequentially removing amino acids from the termini of peptides. This moiety can be ClickP, (as described in U.S. Pat. No. 11,499,979), PITC, dinitrofluorobenzene, dansyl chloride, or other variants. The second component is a DNA sequence onto which a copy of the peptide barcode will be transferred once the BTR reacts to a peptide. The DNA sequence, at a minimum, possesses a sequence that denotes the cycle number during sequencing. Primer sequences for DNA amplification may be added as well. Primer sequences to initialize the copying of the parent peptide barcode may also be included.

Method 1. Preparing DNA-Based BTR that Reacts to N-Termini

The barcode transfer reagent (BTR) is a DNA oligonucleotide with a 5′ 1-(2-azidoethyl)-4-isothiocyanatobenzene (“ClickP”) modification. The DNA oligonucleotide is designed with a 5′ PCR handle, a cycle number barcode (a short sequence indicating the round or cycle number of sequencing) and a 3′ primer region (8-10 bp). The DNA barcode is synthesized with a 5′ DBCO or alkyne modification. Subsequently, copper-catalyzed click chemistry reaction is carried out to conjugate ClickP to the 5′ alkyne. This step yields the functional BTR.

Method 2. Preparing N-Termini Reacting DNA-Based BTR with a Hairpin Blocking Sequence

The barcode transfer reagent (BTR) is a DNA oligonucleotide with a 5′ ClickP modification. To prevent non-specific hybridization of the oligonucleotide in downstream steps, a 3′ hairpin region is added to block the primer region. The DNA oligonucleotide is designed with a 5′ PCR handle, a cycle number barcode (a short sequence indicating the round or cycle number of sequencing), a primer-complementary region (8-10 bp), a 10 bp spacer, and a 3′ primer region. The primer region, which in downstream steps will initiate barcode sequence transfer, is blocked by the complementary region in a hairpin structure. When needed, the primer is accessed by adding a complementary strand-displacing sequence to linearize the hairpin structure.

### Method 3. Preparing DNA-Based BTR on a Bead

The barcode transfer reagent can be prepared on magnetic beads to facilitate the collection of cleaved amino acids. First, the DNA barcode transfer reagent is prepared with a 5′ azide modification, a 5′ adjacent internal amine modification, a 5′ PCR handle, a cycle number barcode (a short sequence indicating the round or cycle number of sequencing) and a 3′ primer region (8-10 bp). BTRs are prepared separately for each cycle number at a concentration of 10 μM in 50 mM borate, pH 8.5. For each cycle number, 1-3 mg of NHS-activated 1 μm magnetic beads (Thermo Fisher Scientific) are prepared in 50 mM borate, pH 8.5. Immediately, 100 nM-1 μM of BTR is added and the reaction incubated at room temperature for 2 hours while shaking. The beads are then washed once with 1M Tris-HCl pH 8, and once with 1× PBS. Subsequently, click chemistry reaction is carried out to conjugate ClickP to the 5′ azide on the BTR by adding 100 μM ClickP to the beads in a buffer for copper-catalyzed click chemistry. After an hour reaction at room temperature, the beads are washed and stored in 1× PBS until use.

### Example 5B. Attaching Barcode Transfer Reagent (BTR) to Peptides

General Principle: The BTR is applied to the peptide-barcode constructs, at which point it will react to the exposed termini of peptides.

### Method 1. Reacting BTR to the N-Termini of DNA-Barcoded Peptides Prepared on Magnetic Beads

The BTR is prepared at 100 μM in Coupling Buffer (acetonitrile:pyridine triethylamine:water at a respective ratio of 5:2:1:3). The magnetic beads with the peptide-barcodes are washed once with Coupling Buffer. Then BTR solution is added to the beads, and the reaction is incubated for 1 hr at 50° C. This step results in the BTR reacting to the N-termini of the peptides attached to the beads. Once the reaction is done, the beads are washed twice with coupling buffer, once with water, and resuspended in 1× NEBBuffer 2 (NEB) or 1× PBS.

### Method 2. Reacting BTR to the N-Termini of DNA-Barcoded Peptides Prepared on Glass Slides

The BTR is prepared at 100 μM in Coupling Buffer (acetonitrile:pyridine triethylamine:water at a respective ratio of 5:2:1:3). Glass slides with the peptide-barcodes are washed once with Coupling Buffer. BTR solution is added to the glass slides, and the reaction is incubated for 1 hr at 50° C. This step results in the BTR reacting to the N-termini of the peptides attached to the glass slides. Once the reaction is done, glass slides are washed twice with Coupling buffer, once with water, and stored in 1× NEBBuffer 2 (NEB) or 1× PBS.

### Method 3. Reacting BTR to the N-Termini of DNA-Barcoded Peptides Prepared in Solution

BTR is prepared at 100 μM in Coupling Buffer (acetonitrile:pyridine:triethylamine water at a respective ratio of 5:2:1:3), and to this solution barcoded peptides are added to a final concentration of 10 μM. The reaction is incubated for 1 hr at 50° C. Solution is evaporated with Argon or N2. Rinsed with ethyl ether to remove excess coupling buffer. The purified BTR-Peptide-barcode product is suspended in 1× NEBBuffer 2 (NEB) or 1× PBS.

### Method 4. Sequentially Reacting Components of BTR to the N-Termini of DNA-Barcoded Peptides Prepared on Magnetic Beads

ClickP is prepared at 100 μM in Coupling Buffer (acetonitrile:pyridine triethylamine:water at a respective ratio of 5:2:1:3). The magnetic beads with the peptide-barcodes are washed once with Coupling Buffer. Then the ClickP solution is added to the beads, and the reaction is incubated for 1 hr at 50° C. This step results in ClickP reacting to the N-termini of the peptides attached to the beads. Subsequently, the beads are washed and moved into a buffer for copper-catalyzed click chemistry. The BTR DNA oligonucleotide is prepared with a 5′ azide modification, PCR handle, a cycle number barcode (a short sequence indicating the round or cycle number of sequencing) and a 3′ primer region (8-10 bp). The BTR DNA oligo is added to the beads at a final concentration of 1-10 μM and the reaction incubated for 1 hr at room temperature. Once the reaction is done, the beads are washed once with 1× PBS, once with water, and resuspended in 1×NEBBuffer 2 (NEB).

### Example 6. Copying Peptide Barcode to BTR

General Principle: Once the BTR is reacted to the terminus of a peptide-barcode construct, the peptide barcode is then enzymatically copied over to the BTR.

### Method 1. Polymerase Mediate Barcode Information Transfer for Samples on a Magnetic Bead Support

A solution is prepared with 0.5 U/μl DNA Polymerase I, Klenow Fragment (NEB), 250 μM dNTPs in 1× NEBBuffer 2. This mixture is then added to the magnetic beads with BTR-peptide-barcode complex. The reaction is incubated at 37° C. for 30 minutes. When the reaction is completed, the beads are washed once with 1× NEBBuffer 2, and once with deionized water, and twice with acetonitrile.

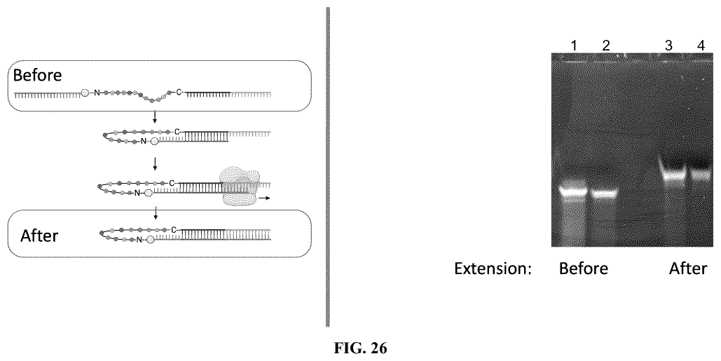

FIG. 26 shows example data of an experiment showing transfer of a peptide barcode to a BTR. A peptide comprising an azide group conjugated to a lysine at the N-terminus (sequence of the peptide from N-terminus to C-terminus is K{azide}}FGGGRGRGR (SEQ ID NO: 1)) is conjugated at the C-terminus to a peptide nucleic acid barcode molecule (peptide barcode) using EDC coupling chemistry. The peptide nucleic acid barcode molecule has the sequence AAAAAAAAAAAAAAAAAAAGGAAGGAGAGGGAAA/3AmMC6T/(SEQ ID NO: 2). The N-terminal azide group of the peptide-barcode construct is also reacted with two different DBCO-conjugated nucleic acid barcode molecules (BTR barcodes) that contain either (i) 21 nucleotides (/5DBCOTEG/TTTTTTTTTCCCTCTCCTTCC (SEQ ID NO: 3)) or (ii) 15 nucleotides (/5DBCOTEG/TTTCCCTCTCCTTCC (SEQ ID NO: 4)). The DBCO of the BTR barcodes is reacted with the azide of the peptide in a click chemistry reaction to generate a complex comprising (A) the BTR barcode, where the barcode has either sequence (i) or (ii) above, (B) the peptide, and (C) the peptide barcode. In some examples, the peptide barcode may be conjugated to the peptide, and then the BTR barcode may be conjugated to the peptide-barcode complex. Alternatively, the peptide barcode may be conjugated to the peptide subsequent to conjugation of the BTR barcode to the peptide. The peptide barcode comprises a sequence that is complementary to a portion of the BTR barcodes (i and ii), and the complementary sequences are allowed to anneal by heating at 95 degrees Celsius for 1 minute followed by slow cooling to 46 degrees Celsius in 1×Taq PCR buffer. An extension reaction is performed using a polymerase (Taq DNA polymerase at 50 degrees Celsius for 10 min and 68 degrees Celsius for 10 min) and the addition of dNTPs (thymines) to extend the BTR barcode, thereby copying the rest of the peptide barcode on to the BTR barcode. The samples are then denatured and electrophoresed in a DNA gel and stained. The lanes of the gel (1, 2, 3, and 4) show the complexes prior to extension (lane 1 is run using the BTR barcode (i) and lane 2 is run using the BTR barcode (ii)), and subsequent to extension (lane 3 is run using the extended BTR barcode (i) and lane 4 is run using the BTR barcode (ii)). As can be observed, the extension results in a larger DNA product (lower electrophoretic mobility), showing successful extension of the peptide barcode (complement) on the BTR barcode.

### Method 2. Ligation and Cleavage Based Barcode Information Transfer for Samples on a Magnetic Bead Support

A solution is prepared with 10U of CircLigase II ssDNA Ligase (Lucigen) in 0.033 M Tris-acetate (pH 7.5), 0.066 M potassium acetate, 0.5 mM DTT, and 2.5 mM Manganese chloride. The mixture is then added to the magnetic beads with BTR-peptide-barcode complex and incubated at 37° C. for 30 minutes. The beads are then washed with 1× CutSmart Buffer (NEB). Then, an oligonucleotide is added containing regions complementary to both the BTR as well as peptide barcode, and a NotI restriction site. The oligonucleotide is added at a final concentration of 10 nM along with 1U of NotI restriction enzyme. The reaction is incubated 37° C. for 30 minutes. When the reaction is completed, the beads are washed once with 1×CutSmart buffer, and once with deionized water.

### Example 7. Cleavage of BTR and Terminal Amino Acid from Peptides

General Principle: After the transfer of the peptide barcode information to the BTR, the BTR is then reacted (e.g., with addition of acid) to cleave the terminal amino acid to which it is attached. This step releases the BTR-terminal amino acid complex (BTR-AC). The ability to cleave is dictated by the reactive moiety of the BTR, and the cleavage itself can be induced chemically or enzymatically. Furthermore, the liberated BTR-ACs can either be released into solution or collected on a solid support.

Method 1. Chemical Based Cleavage of BTR from Barcoded Peptides on Magnetic Beads

The acetonitrile solvent is removed from the magnetic beads and 100% Trifluoroacetic acid (TFA) is added. The mixture of the beads and TFA is incubated for 30 minutes at 50° C. This chemical step liberates the BTR along with the N-terminal amino acid. When the reaction is completed, the beads are pulled down and the supernatant is transferred to a new vial. The beads are then washed once with ethyl acetate, once with water, and once with Coupling Buffer (acetonitrile:pyridine:triethylamine:water at a respective ratio of 5:2:1:3). The next round or cycle of sequencing can now resume starting from Step 4.

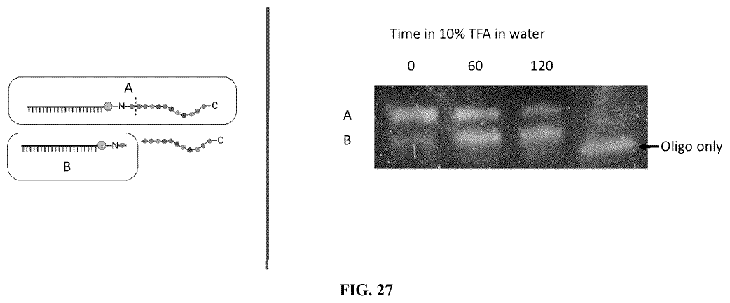

FIG. 27 shows example data demonstrating chemical cleavage of a BTR-AC from the peptide to which it is tethered. A peptide comprising 10 amino acids of sequence WDGGGRGRGR (SEQ ID NO: 5) (from N-terminus to C-terminus) was synthesized. The peptide is reacted with a BTR precursor, 1-(2-azidoethyl)-4-isothiocyanatobenzene, comprising a phenylisothiocyanate group that reacts with N-terminal amino acids, to generate a BTR-AC precursor complex. The BTR precursor of the BTR-AC precursor complex is reacted with a DBCO-conjugated nucleic acid barcode molecule (BTR barcode) containing 21 nucleotides (/5DBCOTEG/TTTTTTTTTCCCTCTCCTTCC (SEQ ID NO: 6)). The DBCO can react with the azide of the BTR precursor in a click chemistry reaction to generate a BTR-AC conjugated to the peptide, where the BTR-AC comprises the BTR barcode. Cleavage is performed by exposing the BTR-AC conjugated to the peptide to 10% trifluoroacetic acid in water for either 0 minutes, 60 minutes, or 120 minutes. The products were then run in a DNA gel using electrophoresis and stained for DNA. As can be seen from the DNA gel of FIG. 27, each lane (column) represents a different treatment condition (duration of 10% TFA treatment). The first row (“A”) represents the uncleaved product (BTR-AC conjugated to the peptide), and the second row (“B”) represents the cleaved BTR-AC. The fourth lane (unlabeled) was loaded with only the DBCO-conjugated nucleic acid barcode molecule (negative control). As can be observed in FIG. 27, the TFA cleavage results in some successful cleavage of the BTR-AC from the peptide, with qualitatively improved cleavage for longer cleavage durations.

Method 2. Enzymatic Cleavage of BTR from Barcoded Peptides on Magnetic Beads

Beads bearing barcoded peptides reacted to BTRs are first washed and placed in 0.1M Sodium Acetate pH 5.5, 1 mM DTT, 0.01% TritonX. Then, edmanase enzyme (Borgo et al, 2015) is added to a final concentration of 100 nM and the reaction is incubated for 2 hours at 30° C. When the reaction is completed, the beads are pulled down and the supernatant is transferred to a new vial. The beads are then washed once with ethyl acetate, once with water, and once with Coupling Buffer (acetonitrile:pyridine:triethylamine:water at a respective ratio of 5:2:1:3). The next round or cycle of sequencing can now resume starting from Step 4.

### Method 3. Chemical Cleavage and Local Retention of BTR on Magnetic Beads

After transfer of peptide barcode to BTR, the cleaved BTR can be retained on the same magnetic bead support as its parent peptide. In this approach the magnetic beads bearing the peptide-barcode constructs also contain an anchor DNA sequence to retain cleaved BTRs. First, the 3′ end of the BTR is ligated to the 5′ phosphate of the anchor DNA sequence via a splint DNA sequence that bridges both sequences as follows. The beads are pulled down and washed once with 1× T4 DNA Ligase Buffer (NEB). Then, a solution is added with 400 units of T4 DNA Ligase (NEB) and 100 nM of Splint oligo in 1×T4 DNA Ligase buffer. The mixture is incubated at 16° C. for 2 hours. After completion, the beads are washed once with deionized water and twice with acetonitrile.

The acetonitrile solvent is removed from the magnetic beads and 100% Trifluoroacetic acid (TFA) is added. The mixture of the beads and TFA is incubated for 30 minutes at 50° C. This chemical step cleaves the BTR along with the N-terminal amino acid, while the BTR-terminal amino acid complex remains attached to the same magnetic bead via the anchor DNA sequence. The beads are then washed once with ethyl acetate, once with water, and once with Coupling Buffer (acetonitrile:pyridine:triethylamine:water at a respective ratio of 5:2:1:3). The next round or cycle of sequencing can now resume starting from Step 4.

### Example 8. Collecting Cleaved Btr and Terminal Amino Acid Complex

General Principle: The cleavage of the BTRs liberates the BTR-terminal amino acid complexes (BTR-AC) from the peptide-barcode constructs and releases them into solution. If the peptide-barcode constructs are on a solid support, such as beads, they can be easily separated from the supernatant and prepared for the next round or cycle of sequencing. BTR-ACs in solution need to be purified for the subsequent steps. If the BTR is composed of DNA, then the BTR-ACs can be purified using common methods for DNA purification, such as ethanol precipitation or bead pull down.

Method 1. Collecting and Purifying Cleaved BTR after TFA Cleavage Via Ethanol Precipitation

The TFA supernatant now contains the cleaved BTR-terminal amino acid complexes (BTR-AC) liberated from individual peptides. The BTR-ACs also contain a copy of their respective peptide barcodes. BTR-ACs are purified from the TFA solvent via ethanol precipitation as follows. The TFA supernatant is diluted 1:10 in water. Then, 1:1000 dilution of glycogen (5 mg/ml, thermo fisher) is added followed by 1:10 volume of 3M Sodium Acetate and mixed. 4 volumes of ice cold 100% ethanol is added and mixed. The mixture is then placed at-20° C. for 1 hr or overnight. During this period, the BTR-ACs will form precipitates. After the incubation, the mixture is spun at 13,000 r.p.m. at 4° C. for 30 minutes. The precipitate will be visible as a pellet. The pellet is washed once with ice cold 80% ethanol. The ethanol is then removed from the pellet, and the pellet is allowed to air dry. Finally, the dried pellet is resuspended in 1× PBS and the concentration determined with a UV-VIS spectrometer.

Method 2. Collecting and Purifying Cleaved BTR after Enzymatic Cleavage Via Bead-Based Pull Down

Magnetic beads are first prepared labeled with DNA sequences complementary to the primer and cycle sequences of the BTR. Then, 0.5-1 mg of these beads are added to the supernatant of edmanase cleavage reaction that contains the cleaved BTR-terminal amino acid complexes (BTR-AC) liberated from individual peptides. The mixture is then incubated for 4 hours at 37° C. The magnetic beads are pulled down and the supernatant is removed. Then, deionized water is added to the samples and the BTR-ACs are eluted by incubating at 80° C. for 1 hr.

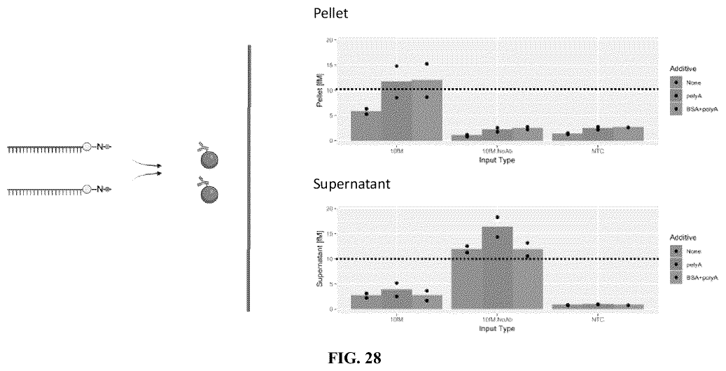

FIG. 28 shows example data from a bead-based pulldown assay. A partially double-stranded oligonucleotide BTR barcode comprising a first strand having the sequence /5FluorT/ACCACAGTCCATGCCATCACTTTCCCTCTCCTTCCCTTGGGTGGAGAGGCT ATTCTACAGCAACAGGGTGGTGGACGCAAATGGGCGGTAGGCGTG/3Phos/(SEQ ID NO: 7) is provided. The BTR barcode comprises a fluorophore (fluorescein) that is used as a model of a peptide. Altogether, the BTR barcode comprising fluorescein is used as a model of a BTR-AC conjugated to a peptide. 10 femtomolar (fM) of the fluorescein BTR barcode is reacted with 40 nanomolar (nM) of anti-fluorescein antibodies comprising biotin moieties and allowed to bind for 90 minutes. The antibodies are then pulled down using streptavidin beads. The beads are then pelleted and the supernatant and beads are separated for further analysis. Further analysis includes performing qPCR on the collected beads as well as the collected supernatant using primers that can anneal to the ACCACAGTCCATGCCATCAC (SEQ ID NO: 8) (forward) and TCCACCACCCTGTTGCTGTA (SEQ ID NO: 9) (reverse) sequences of the BTR barcode first strand or second strand, respectively. Additives such as polyA sequences and bovine serum albumin (BSA) are added to prevent nonspecific binding or loss of DNA (e.g., to tubes, pipette walls, etc.). The bar graphs of FIG. 28 show the amount of DNA obtained, as calculated from qPCR, from the pellet (top) or the supernatant (bottom). The left-most bars represent the amount of DNA obtained from qPCR using 10 fM of the fluorescein BTR barcode and 40 nM of anti-fluorescein antibody when (i) no additive is added (ii) polyA is added and (iii) polyA and BSA are added. The middle set of bars represent the amount of DNA obtained from qPCR when no antibody is added (negative control) and when (i) no additive is added (ii) polyA is added and (iii) polyA and BSA are added, which should result in no capture of the oligos. The right-most bars represent the no template control, with no BTR barcode added but with 40 nM antibody added (negative control) when (i) no additive is added (ii) polyA is added and (iii) polyA and BSA are added. As can be seen from FIG. 28, the quantity of DNA is higher on the pellet as compared to the two negative control conditions when both the antibody and BTR barcode are present, with the additives increasing the quantity of DNA detected. Little to no DNA is detected in the two negative control conditions, as is expected. Similarly, the supernatant has the highest quantity of DNA in the no-antibody control, indicating that the BTR barcodes are present in the solution (supernatant) but not pulled down in the absence of a binding agent (antibodies). Altogether, these results indicate that successful pulldown of fluorescein-BTR barcodes is possible, which suggest that BTR-ACs that are cleaved from a peptide, as described herein, may feasibly be pulled down and detected with high sensitivity.

### Example 9a. Preparing Amino Acid-Specific Binding Agents

General Principle: BTR-ACs are sorted or interrogated based on the identity of the cleaved terminal amino acid. The identity of the terminal amino acids is determined using affinity binding agents, such as antibodies, nanobodies, proteins, or aptamers, which have been designed to recognize these amino acids in a complex with the BTR. These affinity binding agents can be labeled with DNA barcodes, so that during sequencing the identity of an amino acid can be inferred from the DNA barcode of the affinity binding agent to which it is bound. In addition, placing these binding agents on a solid support, such as beads, facilitates pull down and sorting of BTR-ACs.

Method 1. Preparing Amino Acid and PTM Specific Antibodies with DNA Barcodes on Magnetic Beads

Pooling BTR-ACs requires magnetic beads functionalized with amino acid specific antibodies. Given the use of over 20 different amino acid specific antibodies as well as antibodies for post-translational modifications, beads with each type of antibody will be prepared and barcoded with a unique DNA barcode. First, for each amino acid specific primary antibody, DBCO-modified secondary antibodies are prepared by reacting DBCO-PEG4-NHS (Jena Bioscience) with secondary antibodies at a molar ratio 1:10 according to the manufacturer instructions. Then, azide-modified 1 μm magnetic beads (Jena Bioscience) are reacted with DBCO-PEG4-NHS modified secondary antibodies at a molar ratio of 1:100 in 1× PBS for 30 minutes at room temperature. Reacted beads are pulled down and unreacted secondary antibodies are washed away. DNA oligonucleotides containing sequences denoting each type of amino acid-specific antibody are synthesized with 3′ DBCO and 5′ phosphate modifications (Integrated DNA Technologies). Beads conjugated to secondary antibodies are then reacted to their respective amino acid-specific DNA barcodes at a ratio of 1:100. After a magnetic pull-down step, unreacted DNA barcodes are washed away. Finally, for each amino acid specific antibody, primary antibodies are added to their respective secondary antibody conjugated and barcoded beads. After a 10-minute reaction where the primary antibodies bind to their respective secondary antibodies, the fully functionalized beads are pulled down and washed with 1× PBS. At this stage, this preparation should result in a set of magnetic beads each modified with unique amino acid-specific antibodies as well as their respective DNA barcodes.

### Method 2. Preparing Amino Acid and PTM Specific Antibodies Directly Conjugated to DNA Barcodes

Primary antibodies targeting amino acids and PTMs are first prepared in an azide free buffer at a concentration of 0.1-1 mg/ml. Each primary antibody is functionalized with a DBCO moiety by reacting with DBCO-PEG4-NHS (Jena Bioscience) at a molar ratio of 1:10 for 1 hr at room temperature. DNA oligonucleotides containing sequences denoting each type of amino acid-specific antibody are synthesized with 3′ Azide and 5′ phosphate modifications (Integrated DNA Technologies). Each DBCO functionalized antibody is then reacted with its respective DNA barcode at a molar ratio of 1:100 for 1 hr at room temperature. DNA barcoded antibodies are then purified using Amicon Centrifugal Filters (EMD Millipore).

### Example 9B. Pooling and Sorting of BTR-ACS

General Principle: The BTR-ACs, once cleaved from their peptides of origin, exist as a mixture of different BTRs with different cleaved terminal amino acids from different peptides. These BTR-ACs need to be identified or sorted based on the cleaved terminal amino acid each possesses. Affinity binding agents can be deployed in a variety of ways to identify terminal amino acids. In the simplest implementation, a solution of BTR-ACs is exposed to a set of binding agents that identify specific amino acids in a sequential fashion. That way, the first set of binding agents the solution of BTR-ACs is exposed to pull down the amino acid that it recognizes, and the next set does the same, and the process continues until all the BTR-ACs have been pulled down by their respective binding agents and sorted. Alternatively, proximity ligation is used to associate BTR-ACs in solution with a set of binding agents comprising binding agent barcodes such as DNA ID tags. Only when a BTR-AC binds to its binding agent will the ligation of the BTR barcode to the DNA tag of the binding agent occur. PCR is then used to detect the result of this ligation and infer which BTR-ACs are bound to which binding agent.

### Method 1. Binding of BTR-ACs to DNA Barcoded Amino Acid Binding Agents on Magnetic Beads Followed by Proximity Ligation and PCR Amplification

Magnetic beads with different amino acid-specific primary antibodies are combined for a final amount of 1 mg in 500 μl of 1× PBS. BTR-ACs suspended in 1× PBS are added to the bead mixture and incubated for 2 hours at 4° C. The beads are pulled down and washed once with 1×T4 DNA Ligase Buffer (NEB). Then, a solution is added with 400 units of T4 DNA Ligase (NEB) and 100 nM of Splint oligonucleotide in 1×T4 DNA Ligase buffer. The mixture is incubated at 16° C. for 2 hours. The splint oligonucleotide is a short piece of DNA that bridges the barcode region of the BTR-ACs and the amino-acid binding agent DNA barcode on the magnetic beads. When a BTR-AC binds to its respective antibody binding agent on a bead, the splint oligonucleotide enables ligation of the BTR-AC to the amino-acid binding agent DNA barcode on the bead. Once the incubation is completed, ligated BTR-AC and amino-acid DNA barcodes are PCR amplified using KAPA HiFi Polymerase following the manufacturer's instructions. Magnetic beads are pulled down, and the supernatant is collected and PCR amplicons are purified using DNA purification columns (Zymo Research) and suspended in deionized water.

### Method 2. Sequential Sorting of BTR-ACs Using Amino Acid Binding Agents on Magnetic Beads

Amino acid specific primary antibodies attached to magnetic beads are prepared separately in microcentrifuge tubes in 1× PBS. BTR-ACs suspended in 1× PBS are added to one tube and incubated for 2 hours at 4° C. The beads are pulled down, and the supernatant is then added to the next tube containing the next set of primary antibodies and incubated for 2 hours at 4° C. This process is repeated until the supernatant has been applied to each tube containing a unique set of primary antibodies. Since each tube contains primary antibodies specific for one type of amino acid or target, this sequential treatment sorts BTR-ACs into each tube depending on the cleaved amino acid. Bound BTR-ACs to primary antibodies on beads are eluted by moving beads into a 5% SDS, 50 mM Tris-HCl pH 6.5 solution and incubating at 50° C. for 10 minutes. BTR-ACs in elutions are then purified via ethanol precipitation.

### Example 10. Read Out of Barcode Transfer Reagent Sequence

General Principle: The results from Example 9 above provide either BTR-ACs sorted into different tubes/compartments based on their amino acid, or BTR-ACs concatenated with a binding agent barcode, e.g., DNA tag, that denotes the identity of their amino acid. Either way, the output is a DNA sequence that will need to be readout, primarily via Next Gen Sequencing.

### Method 1. Next Gen Sequencing Using Lumina NextSeq500 Sequencer

A sequencing library is prepared from purified PCR products using the NEBNext Ultra II DNA library prep kit (NEB) according to the manufacturer instructions. The prepared library is then sequenced on a NextSeq500 sequencer using a NextSeq 500/550 High Output Kit v2.5 to carry out paired-end 150 bp reads following the manufacturer's instructions.

### Method 2. Next Gen Sequencing Using Oxford Nanopore Sequencer

A sequencing library is prepared from purified PCR products through end repair and attachment of adapter sequences using the Ligation Sequencing Kit (Oxford Nanopore) according to the manufacturer instructions. Libraries are then sequenced on a PromethION48 Long Read sequencer following the manufacturer's instructions.

### Method 3. Sequential Hybridization-Based Readout of Barcode Sequences

Here, the barcode sequence of cleaved BTR-terminal amino acid complexes (BTR-ACs) is read out via combinatorial labeling with fluorescent DNA probes followed by imaging by adapting a sequential hybridization decoding method (Lubeck et al. 2014). First, sorted BTR-ACs are equipped with a Rolling Circle Amplification (RCA) primer using PCR. The BTR-ACs are circularized using CircLigase II (Lucigen) according to the manufacturer's instructions. Following this step, RCA is initiated by incubating samples with 1000 U/mL Phi29 polymerase, 250 μM dNTP, 40 μM aminoallyl dUTP in 1×Phi29 buffer at 30° C. for 2 hours. Microscopy coverslips with amine functional groups are prepared. RCA amplicons are purified using gel electrophoresis and mixed with 5 mM BS(PEG)9 (ThermoFisher) in 1×PBS and placed on coverslips and incubated for 2 hours. After incubation, the coverslips are washed with 50 mM Tris pH 8.0 and then moved to 1× PBS.

Decoding oligonucleotide probes that hybridize to BTR-AC barcodes are conjugated with fluorescent dyes (e.g., Alexa488, Alexa 546, Alexa 594, Atto 640). For a round or cycle of hybridization, a set of decoding probes at 100 nM-10 μM is applied to the amplicon bearing coverslips in hybridization buffer (10% dextran sulfate, 10% formamide, 2×SSC) and incubated for 10 minutes at 30° C. Unbound probes are washed with a 10% formamide, 2×SSC solution. The coverslips are imaged on a widefield or confocal microscope using a 20× objective. After imaging, the bound decoding probes are removed by treating the coverslips with 80% formamide and incubating at 60° C. for 20 minutes. This process is repeated with another set of decoding probes followed by imaging. After all the decoding probe sets have been applied and the sample imaged over several round or cycles, the output is a series of images where each BTR-AC is represented as a spot on a coverslip, and each round or cycle of imaging identifies a base of the barcode. By analyzing all series of images, the barcodes of BTR-ACs are decoded.

### Example 11. Reconstruction of Peptide Sequences

General Principle: Once BTR-ACs have been readout and their sequence determined, the next step is to reconstruct the sequences of the peptides from which these BTR-ACs originated. The sequence of a BTR-AC provides the identity of the amino acid, the cycle number in the peptide sequencing round or cycles, and the barcode sequence of the parent peptide. Given an adequate yield in the identification of BTR-ACs, this information is sufficient to computationally reconstruct the sequence of peptides. A de novo approach can be used where this information is used to build the sequences of peptides. Alternatively, the data can be compared to expected results from a proteomic database to infer the identity and sequence of peptides, akin to the common data analysis approaches in mass spectrometry proteomics.

Method 1. De Novo Reconstruction from NGS Reads

Low quality reads are removed using a quality score filter. Reads are then grouped based on peptide barcode sequence; peptide barcode sequences are deemed identical if they are within a Hamming distance of two. Once grouped, the amino acid barcode id and cycle number are used to construct a putative sequence for each peptide. Undetected cycle numbers are recorded as gaps for unidentified amino acids in peptide sequences.

Method 2. Reference Based Peptide Matching from NGS Reads

Peptide sequences are reconstructed by matching NGS read patterns from protein sequencing experiments to simulated NGS patterns expected from a given proteomic database as follows. The human proteome database is downloaded from the UniprotKB database to include reviewed as well as predicted sequences. Digestion of proteins to peptides is simulated to yield a library of peptides. For each peptide, a simulation is carried out modeling BTR binding, barcode transfer, cleavage, and BTR readout assuming a range of efficiencies for each chemical and enzymatic step. This process yields a range of peptide sequencing patterns for each peptide, which is then stored as a database.

Experimental NGS reads are first pre-processed by removing low quality score reads, and then grouped into NGS reads from individual peptides via peptide barcode sequences. For each peptide barcode sequence, a sequencing pattern is generated denoting the identified amino acids as well as the respective cycle numbers. This sequencing pattern is compared to the simulated database of peptide sequencing patterns to find matches. Unambiguous matches result in the peptide identity being assigned directly. If a pattern matches multiple simulated peptides, a graph is generated assigning putative peptides to each pattern, which will be resolved during the protein inference/assembly stage.

### Example 12. Assembly of Putative Peptide Sequences into Proteins

General Principle: Once a set of peptide sequences has been reconstructed from the experimental results, the next step is to assemble full length protein sequences along with isoforms and abundance. Here, a reference-based approach can be used by comparing the putative peptide sequences to a proteomic database to infer the presence of target proteins in the sample; this is the state-of-the-art in Mass Spectrometry proteomics. Alternatively, with a large enough data set, protein sequences can be assembled de novo by adapting assembly approaches used in transcriptomics.

Method 1. Reference Based Assembly of Protein Sequences from Putative Peptide Sequences

Putative peptide sequences are filtered based on completeness and length. Sequences with more than 80% gaps are removed. In addition, sequences less than three amino acids are removed. Then, the filtered sequences used to probabilistically infer the presence of proteins by comparing them against the human proteome using Mass Spectrometry proteomics inference algorithms, such as ProteinProphet (Nesvizhskii, 2003).

Method 2. De Novo Assembly of Protein Sequences from Putative Peptide Sequences

While de novo assembly of transcripts is well established in the field of transcriptomics (Martin et al, 2011), analogous methods for proteomics are lacking. Here, the de novo assembly methods Trinity (Haas et al, 2013) and Plass (Steinegger et al, 2019) are adapted to enable de novo assembly of proteins from putative peptide sequences. Briefly, peptide sequences are first broken up into overlapping k-mer sequences, where k is less than the sequence length of the peptide. Then, overlapping k-mer sequences are concatenated to form long contiguous reads (i.e. contigs). Unique contigs and contigs representing protein isoforms are represented as a De Bruijn graph where the nodes are the contigs and edges the connection between them. For each protein graph, the graph is traversed iteratively between all possible connected nodes to yield fully assembled sequences of protein isoforms. Based on the abundance of the putative peptide sequences, the relative abundance of each protein isoform can be assigned.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-311. (canceled)

312. A method for processing a peptide, comprising:
(a) providing the peptide comprising a terminal amino acid, wherein the peptide is coupled to a barcode molecule;
(b) contacting a Barcode Transfer Reagent (BTR) comprising an additional barcode molecule with the terminal amino acid or a terminal amino acid derivative, thereby generating a barcoded-amino acid complex (BTR-AC);
(c) coupling the additional barcode molecule of the BTR to the barcode molecule that is coupled to the peptide; and
(d) cleaving the terminal amino acid or the terminal amino acid derivative, wherein subsequent to the cleaving, the terminal amino acid or the terminal amino acid derivative is coupled to the barcode molecule that is coupled to the peptide.

313. The method of claim 312, wherein the BTR comprises a chemical moiety that reacts with an amino acid.

314. The method of claim 313, wherein the chemical moiety reacts with an N-terminal amino acid.

315. The method of claim 313, wherein the chemical moiety comprises dinitrofluorobenzene, dansyl chloride, thiocyanate, or an isothiocyanate.

316. The method of claim 315, wherein the chemical moiety is the isothiocyanate, and wherein the isothiocyanate is selected from the group consisting of phenyl isothiocyanate (PITC), 3-pyridyl isothiocyanate (PYITC), 2-piperidinoethyl isothiocyanate (PEITC), 3-(4-morpholino) propyl isothiocyanate (MPITC), 3 (diethylamino)propyl isothiocyanate (DEPTIC), naphthylisothiocyanate (NITC), trimethylsilyl isothiocyanate (TMS-ITC), and acetyl isothiocyanate (AITC).

317. The method of claim 312, wherein the barcode molecule or the additional barcode molecule is a nucleic acid barcode molecule.

318. The method of claim 312, Wherein the coupling of (c) comprises directly coupling the additional barcode molecule of the BTR to the barcode molecule that is coupled to the peptide.

319. The method of claim 318, wherein the directly coupling comprises coupling via a click chemistry reaction.

320. The method of claim 318, wherein the directly coupling comprises coupling via ligation.

321. The method of claim 312, wherein the coupling of (c) comprises performing a polymerase extension reaction.

322. The method of claim 312, wherein the cleaving of (d) comprises a chemical cleavage.

323. The method of claim 322, wherein the chemical cleavage comprises an acidic cleavage or a basic cleavage.

324. The method of claim 312, wherein the cleaving of (d) comprises an enzymatic cleavage.

325. The method of claim 312, further comprising, (e) contacting the cleaved terminal amino acid or terminal amino acid derivative with a binding agent.

326. The method of claim 325, wherein the binding agent is specific to the amino acid residue or derivatized amino acid.

327. The method of claim 325, wherein the binding agent comprises an antibody, antibody fragment, single chain variant fragment, nanobody, tRNA synthetase, or aptamer.

328. The method of claim 325, wherein the binding agent comprises a nucleic acid barcode molecule; and further comprising, coupling the nucleic acid barcode molecule to the BTR.

329. The method of claim 328, wherein the nucleic acid barcode molecule identifies the binding agent.

330. The method of claim 328, wherein the coupling comprises performing a polymerase extension reaction.

331. The method of claim 325, wherein the binding agent comprises a fluorophore.

332. The method of claim 331, further comprising detecting the binding agent using single molecule imaging.

333. The method of claim 312, wherein the peptide is affixed to a substrate.

334. The method of claim 333, wherein the substrate is a bead, a glass surface, a silicon surface, a plastic surface, a filter, a membrane, a silicon wafer chip, a flow through chip, a flow cell, a biochip, a microtiter well, an ELISA plate, a spinning interferometry disc, a nitrocellulose-based polymer surface, a polymer matrix, a nanoparticle, or a microsphere.

335. The method of claim 312, further comprising, subsequent to (d), detecting the cleaved terminal amino acid or terminal amino acid derivative.

336. The method of claim 335, wherein the detecting comprises using a nanopore to identify the terminal amino acid.

337. The method of claim 312, further comprising, repeating (b)-(d).

338. The method of claim 337, wherein the additional barcode molecule of the BTR comprises cycle information.

339. The method of claim 312, wherein the barcode molecule coupled to the peptide identifies the peptide or the sample, partition, or cell from where the peptide originated.

340. The method of claim 312, wherein the peptide is from a cell suspension, culture of cells, tissue sample, bodily fluid, or an environmental sample.

341. The method of claim 312, wherein the barcode molecule is coupled to the peptide at a C-terminal amino acid.


SEQUENCE LISTING (10 sequences)

SEQ ID NO: 1 - protein, 10 aa, synthetic construct
  SITE 1: note = Azidated residue
KFGGGRGRGR

SEQ ID NO: 2 - DNA, 35 bp, synthetic construct
  source 1..35: mol_type = other DNA
  modified_base 35: mod_base = OTHER; note = 3' Amino modifier C6 modified nucleotide
aaaaaaaaaaaaaaaaaaaggaaggagagggaaat

SEQ ID NO: 3 - DNA, 21 bp, synthetic construct
  source 1..21: mol_type = other DNA
  modified_base 1: mod_base = OTHER; note = Dibenzocyclooctyl (DBCO)-Triethylene glycol modified nucleotide
tttttttttccctctccttcc

SEQ ID NO: 4 - DNA, 15 bp, synthetic construct
  source 1..15: mol_type = other DNA
  modified_base 1: mod_base = OTHER; note = Dibenzocyclooctyl (DBCO)-Triethylene glycol modified nucleotide
tttccctctccttcc

SEQ ID NO: 5 - protein, 10 aa, synthetic construct
WDGGGRGRGR

SEQ ID NO: 6 - DNA, 21 bp, synthetic construct
  source 1..21: mol_type = other DNA
  modified_base 1: mod_base = OTHER; note = Dibenzocyclooctyl (DBCO)-Triethylene glycol modified nucleotide
tttttttttccctctccttcc

SEQ ID NO: 7 - DNA, 97 bp, synthetic construct
  source 1..97: mol_type = other DNA
  modified_base 1: mod_base = OTHER; note = 5' Fluorescein modified nucleotide
  modified_base 97: mod_base = OTHER; note = 3' phosphate modified nucleotide
taccacagtccatgccatcactttccctctccttcccttgggtggagaggctattctacagcaacagggtggtggacgcaaatgggcggtaggcgtg

SEQ ID NO: 8 - DNA, 20 bp, synthetic construct
  source 1..20: mol_type = other DNA
accacagtccatgccatcac

SEQ ID NO: 9 - DNA, 20 bp, synthetic construct
  source 1..20: mol_type = other DNA
tccaccaccctgttgctgta

SEQ ID NO: 10 - DNA, 16 bp, synthetic construct
  source 1..16: mol_type = other DNA
  misc_difference 7..10: note = a, c, t, g, unknown or other
aattcgnnnnaattcg